SEQUENCE LISTING (89 sequences)

SEQ ID NO: 1 - DNA, 1398 bp, synthetic construct
  source 1..1398: mol_type = other DNA
atggtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccacctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcatcatggcggacgaggagaagctgccgcccggctgggagaagcgcatgagccgcagctcaggccgagtgtactacgccaaccacatcactaacgccagccagtgggagcggcccagcggcaacagcagcagtggtggcaaaaacgggcagggggagcctgccagggtgaccggctaccggctgttcgagccagatagccctagaactccagtggataaagcaaagctgtcattccaattcccc

SEQ ID NO: 2 - protein, 466 aa, synthetic construct
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSIMADEEKLPPGWEKRMSRSSGRVYYANHITNASQWERPSGNSSSGGKNGQGEPARVTGYRLFEPDSPRTPVDKAKLSFQFP

SEQ ID NO: 3 - DNA, 2112 bp, synthetic construct
  source 1..2112: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttccggctggaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcatcatggcggacgaggagaagctgccgcccggctgggagaagcgcatgagccgcagctcaggccgagtgtactacttcaaccacatcactaacgccagccagtgggagcggcccagcggcaacagcagcagtggtggcaaaaacgggcagggggagcctgccagggtgaccggctaccggctgttcgagccagatagccctagaactccagtggataaagcaaagctgtcattccaattcccc

SEQ ID NO: 4 - protein, 704 aa, synthetic construct
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFRLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSIMADEEKLPPGWEKRMSRSSGRVYYFNHITNASQWERPSGNSSSGGKNGQGEPARVTGYRLFEPDSPRTPVDKAKLSFQFP

SEQ ID NO: 5 - DNA, 2088 bp, synthetic construct
  source 1..2088: mol_type = other DNA
atggtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccacctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcatcatggcggacgaggagaagctgccgcccggctgggagaagcgcatgagccgcagctcaggccgagtgtactacttcaaccacatcactaacgccagccagtgggagcggcccagcggcaacagcagcagtggtggcaaaaacgggcagggggagcctgccagggtgaccggctaccgggagttcgagccagatagccctagaactccagtggataaagcaaagctgtcattccaattccccaggcacgaactcattaaagaaaatatgcggtccaagctctatctcgagggaagtgtgaatggtcatcaatttaagtgtactcatgagggcgaaggtaaaccttacgaaggaaagcaaacgaatcgaataaaggtcgtggaaggcggaccgctcccttttgccttcgatattctggctacacatttcatgtatggatctaaagtctttattaagtatcctgcggatctgccagactactttaagcagagctttccagaaggatttacttgggaacgagtgatggtctttgaggatggaggtgtcctcacagctacgcaagatacttcccttcaagatggagaactgatttacaacgtgaaagtgcgcggtgtcaattttcctgcaaatggtccagtcatgcaaaagaagaccctcggatgggaaccttctacagaaacaatgtatcctgcagatggaggtctcgagggtcggtgtgataaggctcttaaactggtcggaggaggtcatctccatgtgaatttcaaaactacctataaaagcaaaaagcctgtcaaaatgccaggtgtacattatgtcgatcgacggctcgagcggataaaggaagctgataatgaaacgtatgtggaacagtatgaacatgccgtcgcacggtatagtaatctcggtggcggaatggatgaactctataag

SEQ ID NO: 6 - protein, 696 aa, synthetic construct
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSIMADEEKLPPGWEKRMSRSSGRVYYFNHITNASQWERPSGNSSSGGKNGQGEPARVTGYREFEPDSPRTPVDKAKLSFQFPRHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 7 - DNA, 2712 bp, synthetic construct
  source 1..2712: mol_type = other DNA
atggtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccacctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcggcggaggtagtatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggataccggagggagtggaggtggaggctctggaggagggggttcaggtggtggaggcagcggcggaggtggatccggaggaggtggcagtgggggagtgaccggctaccggctgttcgagccatttcgaggccgttctagaaccgctccggctaacctctgggcaaggcacgaactcattaaagaaaatatgcggtccaagctctatctcgagggaagtgtgaatggtcatcaatttaagtgtactcatgagggcgaaggtaaaccttacgaaggaaagcaaacgaatcgaataaaggtcgtggaaggcggaccgctcccttttgccttcgatattctggctacacatttcatgtatggatctaaagtctttattaagtatcctgcggatctgccagactactttaagcagagctttccagaaggatttacttgggaacgagtgatggtctttgaggatggaggtgtcctcacagctacgcaagatacttcccttcaagatggagaactgatttacaacgtgaaagtgcgcggtgtcaattttcctgcaaatggtccagtcatgcaaaagaagaccctcggatgggaaccttctacagaaacaatgtatcctgcagatggaggtctcgagggtcggtgtgataaggctcttaaactggtcggaggaggtcatctccatgtgaatttcaaaactacctataaaagcaaaaagcctgtcaaaatgccaggtgtacattatgtcgatcgacggctcgagcggataaaggaagctgataatgaaacgtatgtggaacagtatgaacatgccgtcgcacggtatagtaatctcggtggcggaatggatgaactctataag

SEQ ID NO: 8 - protein, 904 aa, synthetic construct
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSMDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTGGSGGGGSGGGGSGGGGSGGGGSGGGGSGGVTGYRLFEPFRGRSRTAPANLWARHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 9 - DNA, 2022 bp, synthetic construct
  source 1..2022: mol_type = other DNA
atggtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccacctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcggcggaggtagtatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggataccggagggagtggaggtggaggctctggaggagggggttcaggtggtggaggcagcggcggaggtggatccggaggaggtggcagtgggggagtgaccggctaccggctgttcgagccatttcgaggccgttctagaagtgctccgcctaacctctgggca

SEQ ID NO: 10 - protein, 674 aa, synthetic construct
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSMDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTGGSGGGGSGGGGSGGGGSGGGGSGGGGSGGVTGYRLFEPFRGRSRSAPPNLWA

SEQ ID NO: 11 - DNA, 2736 bp, synthetic construct
  source 1..2736: mol_type = other DNA
atggtgagtaagggcgaggaagtgatcaaagagttcatgcggtttaaggtgagaatggaaggaagcatgaacggccacgagttcgaaattgagggagaaggagagggacggccctacgagggcacccagacagccaagctgaaagtgacaaagggcgggcctctgccattcgcttgggacatcctgagcccacagtttatgtacggctccaaggcctatgtgaaacatccagctgacattcccgattataagaaactgagcttccccgaggggtttaagtgggaaagagtgatgaacttcgaggacggaggcctggtgactgtgacccaggacagctccctgcaggatgggaccctgatctacaaggtgaaaatgagagggacaaattttccccctgatggacctgtgatgcagaagaaaactatgggatgggaggcctccaccgaaaggctgtatccacgcgacggggtgctgaaaggagaaatccaccaggctctgaagctgaaagatgggggacattacctggtggagttcaagacaatctacatggccaagaaacctgtgcagctgccaggctactattacgtggacacaaaactggatatcacttcacacaacgaggactacactattgtggagcagtatgaacggagcgaggggagacaccatctgttcctgggccatgggactggaagtaccggctcagggtctagtggaaccgcctcaagcgaggataacaatatggctgtgatcaaagagttcatgaggtttaaggtgcgcatggagggcagcatgaatgggcacgaatttgagattgaaggagagggcgaagggaggccttacgagggcacacagactgccaagctgaaagtgaccaagggaggaccactgcctttcgcttgggatatcctgtctcctcagtttatgtacggaagtaaggcctatgtcaagcatcccgctgacattcctgattacaagaaactgtctttcccagagggctttaagtgggagagagtgatgaattttgaagatggaggcctggtgaccgtgacacaggactcctctctgcaggatggcactctgatctacaaagtcaaaatgcgcggcaccaattttccacccgatgggcccgtgatgcagaagaaaacaatggggtgggaggccagcactgaacggctgtatcctagagacggagtgctgaagggcgaaatccaccaggccctgaagctgaaagacggcggccactacctggtggagttcaaaaccatctacatggccaagaaaccagtgcagctgcccggctattactatgtggacaccaagctggatatcacatcccacaatgaagactacaccattgtggaacagtatgagaggtctgaaggacgccaccatctgtttcggctggaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcggcggaggtagtatggataaaaatgagctggttcagaaggccaaactggccgagcaggctgagcgatatgatgacatggcagcctgcatgaagtctgtaactgagcaaggagctgaattatccaatgaggagaggaatcttctctcagttgcttataaaaatgttgtaggagcccgtaggtcatcttggagggtcgtctcaagtattgaacaaaagacggaaggtgctgagaaaaaacagcagatggctcgagaatacagagagaaaattgagacggagctaagagatatctgcaatgatgtactgtctcttttggaaaagttcttgatccccaatgcttcacaagcagagagcaaagtcttctatttgaaaatgaaaggagattactaccgttacttggctgaggttgccgctggtgatgacaagaaagggattgtcgatcagtcacaacaagcataccaagaagcttttgaaatcagcaaaaaggaaatgcaaccaacacatcctatcagactgggtctggcccttaacttctctgtgttctattatgagattctgaactccccagagaaagcctgctctcttgcaaagacagcttttgatgaagccattgctgaacttgatacattaagtgaagagtcatacaaagacagcacgctaataatgcaattactgagagacaacttgacattgtggacatcggataccggagggagtggaggtggaggctctggaggagggggttcaggtggtggaggcagcggcggaggtggatccggaggaggtggcagtgggggagtgaccggctaccggctgttcgagccatttcgaggccgttctagaagtgctccgcctaacctctgggca

SEQ ID NO: 12 - protein, 912 aa, synthetic construct
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFRLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSGGGSMDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDTGGSGGGGSGGGGSGGGGSGGGGSGGGGSGGVTGYRLFEPFRGRSRSAPPNLWA

SEQ ID NO: 13 - protein, 228 aa, synthetic construct
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGG

SEQ ID NO: 14 - protein, 469 aa, synthetic construct
MVSKGEEVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFLGHGTGSTGSGSSGTASSEDNNMAVIKEFMRFKVRMEGSMNGHEFEIEGEGEGRPYEGTQTAKLKVTKGGPLPFAWDILSPQFMYGSKAYVKHPADIPDYKKLSFPEGFKWERVMNFEDGGLVTVTQDSSLQDGTLIYKVKMRGTNFPPDGPVMQKKTMGWEASTERLYPRDGVLKGEIHQALKLKDGGHYLVEFKTIYMAKKPVQLPGYYYVDTKLDITSHNEDYTIVEQYERSEGRHHLFRL

SEQ ID NO: 15 - protein, 225 aa, synthetic construct
MVSKGEAVIKEFMRFKVHMEGSMNGHEFEIEGEGEGRPYEGTQTAKLKVTKGGPLPFSWDILSPQFMYGSRAFIKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTG

SEQ ID NO: 16 - protein, 229 aa, synthetic construct
MDSTEAVIKEFMRFKVHMEGSMNGHEFEIEGEGEGRPYEGTQTAKLRVTKGGPLPFSWDILSPQFMYGSRAFTKHPADIPDYWKQSFPEGFKWERVMNFEDGGAVSVAQDTSLEDGTLIYKVKLRGTNFPPDGPVMQKKTMGWEASTERLYPEDVVLKGDIKMALRLKDGGRYLADFKTTYRAKKPVQMPGAFNIDRKLDITSHNEDYTVVEQYERSVARHSTGGSGGS

SEQ ID NO: 17 - protein, 228 aa, synthetic construct
MVSKGEELIKENMRSKLYLEGSVNGHQFKCTAEGEGKPYEGKQTARIKVVEGGPLPFAFDILAQSFMYGSKVFIKYPADLPDYFKQSFPEGFTWERVMVFEDGGVLTATQDTSLQDGELIYNVKLIGVNFPANGPVMQKKTLGWEPSTETMTPADGGLEGRCDKVLKLVGGGYLHVNFKTTYKSKKPVKMPGVHYVDRRLERIKEADNETYVEQYEHAVARYSNLGGG

SEQ ID NO: 18 - protein, 218 aa, synthetic construct
NMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFT

SEQ ID NO: 19 - protein, 239 aa, synthetic construct
MVSKGEELFTGVVPILVELDGDVNGHKFSVRGEGEGDATNGKLTLKFICTTGKLPVPWPTLVTTLGYGVACFARYPDHMKQHDFFKSAMPEGYVQERTISFKDDGTYKTRAEVKFEGDTLVNRIVLKGIDFKEDGNILGHKLEYNFNSHKVYITADKQKNGIKANFKTRHNVEDGGVQLADHYQQNTPIGDGPVLLPDNHYLSHQSKLSKDPNEKRDHMVLKERVTAAGITHDMDELYK

SEQ ID NO: 20 - protein, 157 aa, synthetic construct
FTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 21 - protein, 8 aa, synthetic construct
VTGYREFE

SEQ ID NO: 22 - protein, 8 aa, synthetic construct
VTGYRLFE

SEQ ID NO: 23 - protein, 54 aa, synthetic construct
MADEEKLPPGWEKRMSRSSGRVYYFNHITNASQWERPSGNSSSGGKNGQGEPAR

SEQ ID NO: 24 - protein, 232 aa, synthetic construct
MDKNELVQKAKLAEQAERYDDMAACMKSVTEQGAELSNEERNLLSVAYKNVVGARRSSWRVVSSIEQKTEGAEKKQQMAREYREKIETELRDICNDVLSLLEKFLIPNASQAESKVFYLKMKGDYYRYLAEVAAGDDKKGIVDQSQQAYQEAFEISKKEMQPTHPIRLGLALNFSVFYYEILNSPEKACSLAKTAFDEAIAELDTLSEESYKDSTLIMQLLRDNLTLWTSDT

SEQ ID NO: 25 - protein, 145 aa, synthetic construct
NSMHKFSQEQIGENIVCRVICTTGQIPIRDLSADISQVLKEKRSIKKVWTFGRNPACDYHLGNISRLSNKHFQILLGEDGNLLLNDISTNGTWLNGQKVEKNSNQLLSQGDEITVGVGVESDILSLVIFINDKFKQCLEQNKVDR

SEQ ID NO: 26 - protein, 162 aa, synthetic construct
AGTKGNGRFLTLKPLPDSIIQESLEIQQGVNPFFIGRSEDCNCKIEDSRLSRVHCFIFKKRHAVGKSMYESPAQGLDDIWYCHTGTNVSYLNNNRMIQGTKFLLQDGDEIKIIWDKNNKFVIGFKVEINDTTGLFNEGLGMLQEQRVVLKQTAEEKDLVKKL

SEQ ID NO: 27 - protein, 18 aa, synthetic construct
PDSPRTPVDKAKLSFQFP

SEQ ID NO: 28 - protein, 15 aa, synthetic construct
ISGYLVDSVAKTIDA

SEQ ID NO: 29 - protein, 15 aa, synthetic construct
HDHTGFLTEYVATRW

SEQ ID NO: 30 - protein, 15 aa, synthetic construct
RNSPVTKTPPRDLPT

SEQ ID NO: 31 - protein, 15 aa, synthetic construct
AVIPINGSPRTPRRG

SEQ ID NO: 32 - protein, 15 aa, synthetic construct
ETRSRHSSYPAGTEE

SEQ ID NO: 33 - protein, 15 aa, synthetic construct
ANLNRSTSVPENPKS

SEQ ID NO: 34 - protein, 9 aa, synthetic construct
FTVGTPPSG

SEQ ID NO: 35 - protein, 15 aa, synthetic construct
NFANFSAYPSEEDMI

SEQ ID NO: 36 - protein, 11 aa, synthetic construct
AAEEIYAARRG

SEQ ID NO: 37 - protein, 15 aa, synthetic construct
GIAVPDVYSACKRFE

SEQ ID NO: 38 - protein, 15 aa, synthetic construct
CQSCVSEYKQVGSHP

SEQ ID NO: 39 - protein, 15 aa, synthetic construct
RLIEDNEYTARQGAK

SEQ ID NO: 40 - protein, 19 aa, synthetic construct
PAQPQRRSLRLSAQKDLEQ

SEQ ID NO: 41 - protein, 15 aa, synthetic construct
SETKKGFYSLSVRHR

SEQ ID NO: 42 - protein, 15 aa, synthetic construct
PFRGRSRTAPANLWA

SEQ ID NO: 43 - protein, 15 aa, synthetic construct
RLGERLRTMSCSDKI

SEQ ID NO: 44 - protein, 15 aa, synthetic construct
GQRDRSSSAPNVHIN

SEQ ID NO: 45 - protein, 15 aa, synthetic construct
KDGATMKTFCGTPEY

SEQ ID NO: 46 - protein, 15 aa, synthetic construct
EIRSRHSSYPAGTED

SEQ ID NO: 47 - protein, 15 aa, synthetic construct
PPTVPNDYMTSPARL

SEQ ID NO: 48 - protein, 15 aa, synthetic construct
IPTSAPVYQQPQQQP

SEQ ID NO: 49 - protein, 15 aa, synthetic construct
TFCGTPEYLAPEVLE

SEQ ID NO: 50 - protein, 15 aa, synthetic construct
SKKSRSHSAKTPGFS

SEQ ID NO: 51 - protein, 15 aa, synthetic construct
DPGSAAPYLKTKFIC

SEQ ID NO: 52 - protein, 15 aa, synthetic construct
EILSRRPSYRKILND

SEQ ID NO: 53 - protein, 15 aa, synthetic construct
TEADGELYTFNTPSG

SEQ ID NO: 54 - protein, 15 aa, synthetic construct
DGPKGTGYIKTELIS

SEQ ID NO: 55 - protein, 15 aa, synthetic construct
VTQVSSAYQKTVPVE

SEQ ID NO: 56 - protein, 15 aa, synthetic construct
IIGLQMGTNKFASQQ

SEQ ID NO: 57 - protein, 21 aa, synthetic construct
RLGWWRFYTLRRARQGNTKQR

SEQ ID NO: 58 - protein, 207 aa, synthetic construct
MAKTYDYLFKLLLIGDSGVGKTCVLFRFSEDAFNSTFISTIGIDFKIRTIELDGKRIKLQIWDTAGQERFRTITTAYYRGAMGIMLVYDITNEKSFDNIRNWIRNIEEHASADVEKMILGNKCDVNDKRQVSKERGEKLALDYGIKFMETSAKANINVENAFFTLARDIKAKMDKKLEGNSPQGSNQGVKITPDQQKRSSFFRCVLL

SEQ ID NO: 59 - protein, 200 aa, synthetic construct
MAKKTYDLLFKLLLIGDSGVGKTCVLFRFSDDAFNTTFISTIGIDFKIKTVELQGKKIKLQIWDTAGQERFHTITTSYYRGAMGIMLVYDITNGKSFENISKWLRNIDEHANEDVERMLLGNKCDMDDKRVVPKGKGEQIAREHGIRFFETSAKANINIEKAFLTLAEDILRKTPVKEPNSENVDISSGGGVTGWKSKCC

SEQ ID NO: 60 - protein, 201 aa, synthetic construct
MNPEYDYLFKLLLIGDSGVGKSCLLLRFADDTYTESYISTIGVDFKIRTIELDGKTIKLQIWDTAGQERFRTITSSYYRGAHGIIVVYDVTDQESYANVKQWLQEIDRYASENVNKLLVGNKSDLTTKKVVDNTTAKEFADSLGIPFLETSAKNATNVEQAFMTMAAEIKKRMGPGAASGGERPNLKIDSTPVKPAGGGCC

SEQ ID NO: 61 - protein, 30 aa, synthetic construct
GGSGGGGSGGGGSGGGGSGGGGSGGGGSGG

SEQ ID NO: 62 - protein, 10 aa, synthetic construct
TGYRLFEEIL

SEQ ID NO: 63 - protein, 9 aa, synthetic construct
GYRLFEEIL

SEQ ID NO: 64 - protein, 8 aa, synthetic construct
YRLFEEIL

SEQ ID NO: 65 - protein, 11 aa, synthetic construct
VTGYRLFEEIL

SEQ ID NO: 66 - protein, 10 aa, synthetic construct
VTGYRLFEEI

SEQ ID NO: 67 - protein, 9 aa, synthetic construct
VTGYRLFEE

SEQ ID NO: 68 - protein, 18 aa, synthetic construct
PDVPRTPVDKAKLSFQFP

SEQ ID NO: 69 - protein, 19 aa, synthetic construct
SPDVPRTPVDKAKLSFQFP

SEQ ID NO: 70 - protein, 18 aa, synthetic construct
PDAPRTPVDKAKLSFQFP

SEQ ID NO: 71 - protein, 18 aa, synthetic construct
PDSPRAPVDKAKLSFQFP

SEQ ID NO: 72 - protein, 18 aa, synthetic construct
PDAPRAPVDKAKLSFQFP

SEQ ID NO: 73 - protein, 10 aa, synthetic construct
RVYYFNHITN

SEQ ID NO: 74 - protein, 10 aa, synthetic construct
RVYYANHITN

SEQ ID NO: 75 - protein, 10 aa, synthetic construct
RVYYLNHITN

SEQ ID NO: 76 - protein, 10 aa, synthetic construct
RVYYVNHITN

SEQ ID NO: 77 - protein, 15 aa, synthetic construct
PFRGRSRSAPPNLWA

SEQ ID NO: 78 - protein, 15 aa, synthetic construct
PFRGASRSAPPNLWA

SEQ ID NO: 79 - protein, 15 aa, synthetic construct
PFRGAARSAPPNLWA

SEQ ID NO: 80 - protein, 15 aa, synthetic construct
PFRGRSASAPPNLWA

SEQ ID NO: 81 - protein, 15 aa, synthetic construct
PFRGRSRTAPPNLWA

SEQ ID NO: 82 - protein, 15 aa, synthetic construct
PFRGRSRAAPPNLWA

SEQ ID NO: 83 - protein, 15 aa, synthetic construct
PFRGRSRSAAPNLWA

SEQ ID NO: 84 - protein, 15 aa, synthetic construct
PFRGRSRSAPANLWA

SEQ ID NO: 85 - protein, 15 aa, synthetic construct
PFRGRSASAPANLWA

SEQ ID NO: 86 - protein, 15 aa, synthetic construct
PFRGAARSAPANLWA

SEQ ID NO: 87 - protein, 15 aa, synthetic construct
PFRGRAASAPPNLWA

SEQ ID NO: 88 - protein, 15 aa, synthetic construct
PFRGRAASAPANLWA

SEQ ID NO: 89 - protein, 15 aa, synthetic construct
PFRGRSRAAPANLWA

(19) United States
(12) Patent Application Publication
(10) Pub. No.: US 2026/0077065 A1
(43) Pub. Date: Mar. 19, 2026

(54) Compositions and Methods for Assessing Kinase Activity

(71) Applicant: The Board of Trustees of the Leland Stanford Junior University, Stanford, CA (US)

(72) Inventors: Yichi Su, Redwood City, CA (US); Michael Z. Lin, Redwood City, CA (US); Yan Wu, Redwood City, CA (US)

(21) Appl. No.: 19/110,335

(22) PCT Filed: Sep. 15, 2023
(86) PCT No.: PCT/US2023/074363

§ 371 Date: Mar. 10, 2025

Related U.S. Application Data
(60) Provisional application No. 63/407,091, filed on Sep. 15, 2022.

(51) Int. Cl. A61K 49/00; C12N 9/12; C12N 15/86

(52) U.S. Cl. CPC A61K 49/0008; A61K 49/0021; A61K 49/0045; C12N 9/12; C12N 15/86; C12Y 207/11001; C12N 2740/15043; C12N 2750/14143

(57) ABSTRACT

Provided are nucleic acids encoding kinase-modulated bioluminescent indicator (KiMBI) polypeptides. In certain embodiments, a nuclei acid of the present disclosure encodes a KiMBI polypeptide comprising a first bioluminescent enzyme fragment, a phospho-binding domain, a 5 second bioluminescent enzyme fragment capable of forming an active bioluminescent enzyme with the first fragment via enzyme fragment complementation, and a kinase substrate bound by the phospho-binding domain when phosphorylated. KiMBI polypeptide may be fused to one or more fluorescent proteins, e.g., which exhibit resonance energy transfer (RET). Also provided are KiMBI polypeptides encoded by the nucleic acids of the present disclosure. Cells that express 10 a KiMBI polypeptide are also provided, as are non-human animals comprising such cells. Also provided are methods of assessing activity of a kinase of interest in a non-huma animal, and methods of assessing a test agent for the ability to inhibit a kinase of interest in a non-human animal.

### CROSS-REFERENCE TO RELATED APPLICATIONS

This application claims the benefit of U.S. Provisional Patent Application No. 63/407,091, filed Sep. 15, 2022, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, STAN-1988WO_SEQ_LIST, created on Sep. 14, 2023 and having a size of 98,929 bytes. The contents of the Sequence Listing XML are incorporated herein by reference in their entirety.

### INTRODUCTION

Aberrant kinase activity drives the growth or survival of multiple types of cancers, including brain cancers such as gliomas^{1,2}. Kinase inhibitors have proven to be effective anti-cancer therapies, but so far have only been approved for a subset of extracranial neoplasms. The development of drugs that can effectively inhibit additional kinase targets in cancer remains a high priority in biotechnology.

To create a new kinase inhibitor drug, extensive campaigns are typically undertaken to improve the specificity and pharmacokinetics of lead compounds before efficacy studies can even be attempted. Pharmacokinetics are typically assessed by measuring drug concentrations in plasma at different times after administration. However, drug concentrations in plasma may not reflect intratumor concentrations due to limited vascularization, poor drug permeability, or fast metabolism³. Existing methods for evaluating drug concentrations in tumors involve dissection and chemical analysis, which is expensive, time-consuming, and labor-intensive⁴. Alternatively, drugs can be labelled with a radioactive element, and then their distributions can be imaged by PET or SPECT⁵, but this requires a synthetic pathway to incorporate the isotope without altering the chemical properties of the inhibitor, and is thus even lower-throughput. Assessing target inhibition, which is distinct from drug concentrations, also requires biochemical measurements of kinase or substrate phosphorylation in tissue samples. Thus, a faster and less expensive approach to evaluating drug activity in target tissues would relieve a bottleneck in the discovery of effective kinase inhibitors for cancer therapy.

These challenges for assessing kinase inhibition in vivo are particularly acute for therapeutic indications in the brain. Drug concentrations in plasma differ from concentrations in the brain due to the blood-brain barrier (BBB), so plasma concentrations are even less predictive of brain tumor concentrations. Indeed, most kinase inhibitors demonstrate poor penetration of BBB⁶. Thus, the discovery of effective kinase inhibitors for brain tumors requires medicinal chemistry campaigns with specific assessments of BBB penetration or intratumor concentrations of candidate molecules. An effective approach to quickly and accurately evaluating the efficacy of drug candidates in the brain would be particularly helpful in discovering kinase inhibitors for brain tumors.

### SUMMARY

Provided are nucleic acids encoding kinase-modulated bioluminescent indicator (KiMBI) polypeptides. In certain embodiments, a nucleic acid of the present disclosure encodes a KiMBI polypeptide comprising a first bioluminescent enzyme fragment, a phospho-binding domain, a second bioluminescent enzyme fragment capable of forming an active bioluminescent enzyme with the first fragment via enzyme fragment complementation, and a kinase substrate bound by the phospho-binding domain when phosphorylated. The KiMBI polypeptide may be fused to one or more fluorescent proteins, e.g., which exhibit resonance energy transfer (RET). Also provided are KiMBI polypeptides encoded by the nucleic acids of the present disclosure. Cells that express a KiMBI polypeptide are also provided, as are non-human animals comprising such cells. Also provided are methods of assessing activity of a kinase of interest in a non-human animal, and methods of assessing a test agent for the ability to inhibit a kinase of interest in a non-human animal. In some embodiments, the methods find use in assessing a test agent for the ability to inhibit a kinase of interest in one or more specific target tissues of the non-human animal, such as the brain.

### BRIEF DESCRIPTION OF THE FIGURES

FIG. 1: Development and characterization of PKA KiMBIs. a, The proposed working mechanism of KiMBI. In the presence of active kinase, the interaction between a phosphopeptide-binding domain (PBD) and phosphorylated kinase substrate competes effectively against LgBiT-SmBiT reconstitution (left). Inhibition of kinase allows dephosphorylation of the substrate and loss of binding to the PBD, inducing LgBiT-SmBiT reconstitution (right). b, Bioluminescence of cells expressing putative PKA KiMBIs, differing only by length of the linker between PBD and substrate. All variants show inhibition by the PKA activators Fsk and IBMX.

FIG. 2: Engineering, optimization, and characterization of ERK KiMBIs. Variants were evaluated by measuring the bioluminescence from HEK293A cells transiently expressing caMEK and KiMBI variants and treated with DMSO or MEK inhibitors. The protein domain architecture of the parent construct is shown for each round of optimization. a, Screening of domain orders in the fusion protein architecture. S, SmBiT. b, Domain arrangement and model of an ERK KiMBI. c, Screening of SmBiT truncation variants to destablize LgBiT+SmBiT reconstitution for improving fold induction (from top to bottom and left to right SEQ ID NOs:62-67, 22). d, Left, introduction of a second phosphorylation site for KiMBI optimization, yielding the prototypical construct ΔC3_V-3S. Potential phosphorylation sites for ERK kinase are labeled in red (from top to bottom SEQ ID NOs:68, 69, 27). Right, validation of phosphorylation sites in ΔC3_V-3S. Serine/threonine to alanine mutations were shown in gray (from top to bottom SEQ ID NOs:27, 70-72), e, Screening of mutations in WW domain for enhancing WW+pSub interactions, yielding bKiMBI_{ERK} (from top to bottom SEQ ID NOs:73-76). f, Mean bioluminescence of various cell lines expressing bKiMBI with or without caMEK in response to MEK inhibitor incubation. U87E, U87-EGFRvIII cell line. Unpaired Student's t test was performed. ns, p>0.05; **, p<0.01; ***, p<0.001; **** p<0.0001. Error bars show s.d. g, Mean bioluminescence of HEK293A cells co-expressing caMEK and tKiMBI or tKiMBImut (negative control) in response to inhibitors Vx-11e (ERK, 1 μM), U0126 (MEK, 10 μM), PD0325901 (MEK, 1 μM), GSK1120212 (MEK, 1 μM), SCH772984 (ERK, 1 μM), SP600125 (JNK, 10 μM), and PD169316 (p38, 10 μM). One-way ANOVA analysis followed by Tukey's posthoc test was performed. ns, p>0.05; *, p<0.05.

FIG. 3: Red-shifting bioluminescence signal output of ERK KiMBIs. a, Introducing C-terminal CyOFP1 for red-shifting bioluminescence signal output. b, Bioluminescence spectra of KiMBI variants shown in a, and all spectra were normalized to the peak at around 450 nm. c, Alanine scanning on SmBiT. d, Mutations on SmBiT site 161 & 163. e, Introduction of mutations in SmBiT for improving fold induction of red-shifted KiMBI variants, yielding oKiMBI_{ERK}. f, Top, domain arrangements of orangeKiMBI and tomatoKiMBI. Lower left, bioluminescence spectra of KiMBI variants when fully activated, normalized to the peak at 450 nm. Lower right, mean bioluminescence of the KiMBI variants in response to MEK inhibitor incubation in HEK293A cells transiently co-transfected with caMEK- and KiMBI-encoded plasmids. Signal induction between the DMSO- and inhibitor-treated samples is shown above the bar graphs. Unpaired Student's t test was performed. ns, p>0.05; **, p<0.01; ***, p<0.001; **** p<0.0001. Error bars show s.d. g, Screening of N-terminal fusions of fluorescent proteins.

FIG. 4: Specificity of inhibitors reported by red-shifted KiMBIs. Mean bioluminescence of HEK293A cells co-expressing caMEK and oKiMBI or oKiMBImut as negative control (a), or tKiMBI or tKiMBImut (b), in response to inhibitors Vx-Ile (ERK, 1 μM), U0126 (MEK, 10 μM), PD0325901 (MEK, 1 μM), GSK1120212 (MEK, 1 μM), SCH772984 (ERK, 1 μM), SP600125 (JNK, 10 μM), and PD169316 (p38, 10 μM). Error bars show s.d. One-way ANOVA analysis followed by Tukey's posthoc test was performed. ns, p>0.05; *, p<0.05.

FIG. 5: Comparison between tKiMBI and tKIMBI-duo, a variant with repeated WW domains and substrates. a, Introduction of F25A mutation in WW for improving oKiMBI and tKiMBI. Error bars show s.d. Unpaired Student's t test was performed. ns, p>0.05; *, p<0.05; **, p<0.01; ***, p<0.001; **** p<0.0001. b, Domain structures of tKiMBI reporters. c, Mean bioluminescence of tKiMBI variants in response to MEK inhibitor incubation in HEK293A cells transiently co-transfected with caMEK- and tKiMBI-encoded plasmids. Fold inductions of the signals between the DMSO- and inhibitor-treated samples (left and right, respectively) were shown above the bar graphs. Error bars show s.d. Unpaired Student's t test was performed. ns, p>0.05; **** p<0.0001. d, Kinetics measurement of KiMBIs. Three technical replicates were shown for each group, and the result is a representative of two biological replicates.

FIG. 6: In vitro characterization of U87-EGFRvIII stable reporter cell lines. a, The domain structure of the tKiMBI (or tKiMBImut)-T2A-AkaLuc lentiviral plasmid for generating stable reporter cell lines. BSD, blasticidin-S deaminase, for antibiotic selection (from top to bottom SEQ ID NOs:27, 72):. b, Microscope images of U87-EGFRvIII reporter cell lines stably expressing tKiMBI or tKiMBImut. c, Mean bioluminescence of U87-EGFRvIII stable reporter cells in response to MEK-ERK inhibitors. Fold inductions of the signals between the DMSO- and inhibitor-treated samples were shown above the bar graphs. Bars from left to right are for: +DMSO, +PD0325901, +GSK1120212, and +SCH772984. Error bars show s.d. Unpaired Student's t test. ns, p>0.05; p<0.05; **, p<0.01; ***, p<0.001; **** p<0.0001.

FIG. 7: Molecular imaging of ERK inhibition in a subcutaneous tumor model. a, Scheme of the experimental design. U87-EGFRvIII reporter cells expressing tKiMBI (right) and tKiMBImut (left) were implanted subcutaneously to establish tumors in J:NU mice. AkaLuc imaging with AkaLumine injection was used to monitor tumor growth. To visualize ERK inhibition, the indicators were imaged with FFz injection, 2 h before and after treatment with MEK-ERK inhibitors. b, Bioluminescence imaging of Akaluc at one week after implantation of U87-EGFRvIII reporter cells. c-h, U87-EGFRvIII tumor-bearing mice were sequentially treated (two days apart) with MEK inhibitors PD0325901 (c-e) and GSK1120212 (f-h) and were imaged with FFz injection 2 h after inhibitor injection. c, f, Bioluminescence imaging before and after inhibitor treatment. d, g, Total bioluminescence collected from the tumors. Each line represents a single tumor. e, h, Ratio between the signals collected before and after inhibitor treatment. Each line represents an individual mouse. P values, paired Student's t test.

FIG. 8: Bioluminescence imaging of ERK inhibition in AAV infected mouse brain. a, The domain structures of the tKiMBI- and caMEK-co-expressing AAV plasmids for in vivo gene delivery. b, c, Bioluminescence of HEK293A cells transiently transfected with AAV plasmids in response to MEK-ERK inhibitors incubation. Fold signal induction by each inhibitor relative to DMSO control is indicated above the bars. Bars from left to right are for: +DMSO, +PD0325901, +GSK1120212, and +SCH772984. Error bars show s.d. Unpaired Student's t test. ns, p>0.05; ***, p<0.001; **** p<0.0001. d-g, Raw data of bioluminescence imaging of ERK inhibition in AAV infected mouse brain (FIG. 9). d, f, Bioluminescence imaging before and after inhibitor treatment. e, g, Total bioluminescence collected from the brain tumors. Each line represents an individual mouse. P values, paired Student's t test.

FIG. 9: Molecular imaging of ERK inhibition in AAV infected mouse brain. a, Scheme of AAV infection for KiMBI expression in the mouse striatum. b-d, 4 weeks after AAV infection in the striatum, tKiMBI- or tKiMBImut-expressing J:NU mice were sequentially treated (two days apart) with PD0325901 and GSK1120212 and were imaged with CFz injection. b, Representative bioluminescence images collected before and after inhibitor treatment. c, Ratio between the signals collected before and after PD0325901 (left) or GSK1120212 (right) treatments. P values, unpaired Student's t test. d, Signal ratios comparison between PD0325901 and GSK1120212. Each line represents an individual mouse. P values, paired Student's t test.

FIG. 10: Molecular imaging of ERK inhibition in a brain tumor model. a-d, J:NU Mice with tKiMBI- or tKiMBImut-expressing U87-EGFRvIII tumor engrafted in the striatum were sequentially treated (two days apart) with PD0325901 (a-b) and GSK1120212 (c-d) and were imaged with CFz injection. a and c, Bioluminescence imaging before and after inhibitor treatment. b and d, Ratio between the signal collected before and after inhibitor treatment. P values, unpaired Student's t test. Unpaired Student's t test was performed. ns, p>0.05; ***, p<0.001; **** p<0.0001.

FIG. 11: Molecular imaging of ERK inhibition in brain tumor xenografts. a, Microscope images of LN-229 reporter cell lines stably expressing tKiMBI or tKiMBImut. b, Mean bioluminescence of LN-229 stable reporter cell lines in response to PD0325901 incubation. Bars from left to right are for: +DMSO and +PD0325901. c, Mean bioluminescence of tKiMBI-expressing LN-229 stable cell line in response to MEK-ERK inhibitors. Fold inductions of the signals between the DMSO- and inhibitor-treated samples were shown above the bar graphs. Bars from left to right are for: +DMSO, +PD0325901, +GSK1120212, and +SCH772984. Error bars show s.d. Unpaired Student's t test. ns, p>0.05; *, p<0.05; **, p<0.01; ***, p<0.001. d-g, J:NU Mice with tKiMBI- or tKiMBImut-expressing LN-229 tumor engrafted in the striatum were sequentially treated (two days apart) with GSK1120212 (d-e) and PD0325901 (f-g) and were imaged with CFz injection. d, f, Bioluminescence imaging before and after inhibitor treatment. e, g, Ratio between the signal collected before and after inhibitor treatment. P values, unpaired Student's t test.

FIG. 12: Time-course molecular imaging of ERK inhibition in brain tumor xenografts. a, Scheme of the experimental design of time-course ERK inhibition monitor. The initial bioluminescence signal L₀ was measured with CFz injection 2 hrs before inhibitor treatment. After inhibitor administration, various time points (1, 2, 4, 8, 24 hrs) were taken for measuring the bioluminescence signal L from the xenografted brain tumors. b, Mean normalized signal (L/L₀) collected from reporter-expressing brain tumors over time after treatment with PD0325901. c, Bioluminescence imaging of J:NU mice bearing reporter-expressing U87-EGFRvIII brain tumors over time after treatment with PD0325901 (raw images of b).

FIG. 13: Development and characterization of Akt KiMBI. a. Proposed mechanism of Akt KiMBI. With active Akt, the interaction between phosphorylated kinase substrate (pSub) and phosphopeptide-binding domain (PBD) 14-3-3 outcompetes the weaker LgBiT-SmBiT interaction (left). Upon inhibitor treatment, the dephosphorylation of kinase substrate allows the reconstitution of LgBiT-SmBiT, thus producing light (right). b. Top, domain arrangement of blueKiMBI (bKiMBI) and orangeKiMBI (oKiMBI). Bottom left, bioluminescence spectra of bKiMBI and oKiMBI in HEK293A cells, normalized to the 450-nm peak. Bottom right, bioluminescent signals of of bKiMBI and oKiMBI in response to DMSO (left) or GDC-0068 (10 mM)(right) in HEK293A cells, with caAkt co-transfected. ALU, arbitrary luminescence units. c. Signal increase of oKiMBI (normalized to negative control oKiMBImut) induced by GDC-0068 (10 mM), or M2698 (10 mM) in HEK293A cells, U87-EGFRvIII cells, or LNCaP cells, with or without caAkt co-transfection. Bars from left to right are for: DMSO, GDC-0068 and M2698. d. Signal increase of BAR (normalized to negative control BARmut) induced by GDC-0068 (10 mM), or M2698 (10 mM) in HEK293A cells, U87-EGFRvIII cells, or LNCaP cells, with or without caAkt co-transfection. Bars from left to right are for: DMSO, GDC-0068 and M2698. *, p<0.05; **, p<0.01; ***, p<0.001; **** p<0.0001 by unpaired two-tailed Student's t-test. Error bars, SDs. ALU, arbitrary luminescence units.

FIG. 14: Engineering, optimization, and characterization of Akt KiMBIs. a. Screening of topologies, SmBiT, and substrates. HEK293A cells were transfected with each variant and caAkt, and their luminescent signals were measured after the treatment of DMSO or Akt inhibitor GDC-0068 (10 mM). Bars from left to right are for: DMSO and GDC-0068. b. Screening of linker lengths between 14.3.3 and SmBiT, ranging from 0 to 30 amino acids, yielding bKiMBI_{Akt}. Bars from left to right are for: DMSO and GDC-0068. c. Replacing the N-terminal CyOFP1 with tdTomato (tKiMBI_{Akt}0.1) or introducing a C-terminal CyOFP (oKiMBI_{Akt}0.1) for red-shifting spectra. Bars from left to right are for: DMSO and GDC-0068. d. Introduction of single mutations in kinase substrate for improving inducibility of oKiMBI_{Akt}0.1 (from top to bottom SEQ ID NOs:77-84). Bars from left to right are for: DMSO and GDC-0068. e. Introduction of combinatorial mutations (from top to bottom SEQ ID NOs:77, 85-88, 42) in kinase substrate for improving inducibility of oKiMBI_{Akt}0.1, yielding oKiMBI_{Akt}. Bars from left to right are for: DMSO and GDC-0068. f. luminescent signals of oKiMBI_{Akt} (top) or oKiMBI_{Ak}tmut (bottom) in response to DMSO or GDC-0068 (10 mM) in HEK293A cells, U87-EGFRvIII cells, or LNCaP cells, with or without caAkt co-transfection. Bars from left to right are for: DMSO and GDC-0068. **, p<0.01; ***, p<0.001; **** p<0.0001 by unpaired two-tailed Student's t-test. Error bars, SDs. ALU, arbitrary luminescence units.

FIG. 15: Comparison of Akt KiMBI and BAR. a-b. Luminescent signal increase of oKiMBI and its negative control oKiMBImut (a), or BAR and its negative control BARmut (b) induced by GDC-0068 (10 mM) or M2698 (10 mM) in HEK293A cells with caAkt co-transfection (left), U87-EGFRvIII cells (middle), or LNCaP cells (right). Bars from left to right are for: DMSO, GDC-0068 and M2698. *, p<0.05; **, p<0.01; ***, p<0.001; **** p<0.0001 by unpaired two-tailed Student's t-test. Error bars, SDs. ALU, arbitrary luminescence units.

FIG. 16: Molecular imaging of Akt inhibition in a subcutaneous tumor model. a. Scheme of the experimental design. U87-EGFRvIII cells expressing oKiMBImut (left side) and oKiMBI (right side) were subcutaneously implanted in J:NU mice. After 1-3 weeks, the bioluminescence of tumor-bearing mice was imaged with intraperitoneal (i.p.) FFz injection 2 hours before and 2 hours after oral Akt inhibitor treatment (p.o.). b. Scheme of Akt signaling pathway and the response of oKiMBI to Akt inhibition. c-d. Bioluminescence of oKiMBImut or oKiMBI-expressing tumors before and 2 hours after GDC-0068 (c) or M2698 (d) treatment. Left, representative bioluminescence images acquired before and after drug treatment. Right, responses of oKiMBImut and oKiMBI to drug treatment. Each line represents an individual mouse. P values, by paired two-tailed Student's t-test. L₀, luminescence before drug treatment. ΔL, luminescence change after drug treatment.

FIG. 17: In vitro characterization of KiMBI-expressing reporter cells. a. Domain structure of oKiMBI and oKiMBImut in the lentiviral vector pLL3.7m. BSD, blasticidin-S deaminase, for antibiotic selection (from top to bottom SEQ ID NOs:42, 89). b. U87-EGFRvIII cells stably expressing oKiMBI or oKiMBImut. c. Luminescence of oKiMBI or oKiMBI-expressing U87-EGFRvIII cells in response to GDC-0068 (10 mM) or M2698 (10 mM). Bars from left to right are for: DMSO, GDC-0068 and M2698. *, p<0.05; **, p<0.01; ***, p<0.001; **** p<0.0001 by unpaired two-tailed Student's t-test. Error bars, SDs. ALU, arbitrary luminescence units.

FIG. 18: Molecular imaging of Akt inhibitor activity and pharmacodynamics in mouse brain. a. Scheme of injection of KiMBI-encoding AAVs and hyperactivation of Akt signaling pathway induced by constitutively active Akt (caAkt). b. Bioluminescence of oKiMBImut or oKiMBI-transduced brain before and 3 hours after GDC-0068 treatment. Left, representative bioluminescence images acquired before and after drug treatment. Right, responses of oKiMBImut and oKiMBI to drug treatment. Each dot represents an individual mouse. P values, by unpaired two-tailed Student's t-test. Error bars, SDs. L₀, luminescence before drug treatment. ΔL, luminescence change after drug treatment. c. Molecular imaging of GDC-0068 pharmacodynamics in AAV-transduced brain. Left, representative bioluminescence images acquired before and at different time points after drug treatment. Right, time courses of luminescence in oKiMBI or oKiMBImut-expressing mice.

FIG. 19: Engineering and characterization of KiMBI-encoding AAV. a. Domain structures of oKiMBI and caAkt co-expressing AAV plasmids for in vivo gene delivery. b. Luminescence of different AAV plasmids in response to DMSO or GDC-0068 in HEK293A cells. Top, no filter was applied; bottom, a 610 nm longpass filter was applied to only collect red light. Bars from left to right are for: DMSO and GDC-0068. *, p<0.05; **, p<0.01; ***, p<0.001; **** p<0.0001 by unpaired two-tailed Student's t-test. Error bars, SDs. ALU, arbitrary luminescence units. c. Bioluminescence spectra of oKiMBI and oKiMBImut AAV plasmids in HEK293A cells with DMSO or GDC-0068 treatment, normalized to the 450-nm peak. Bars from left to right are for: DMSO and GDC-0068.

### DETAILED DESCRIPTION

Before the compositions and methods of the present disclosure are described in greater detail, it is to be understood that the compositions and methods are not limited to particular embodiments described, as such may, of course, vary. It is also to be understood that the terminology used herein is for the purpose of describing particular embodiments only, and is not intended to be limiting, since the scope of the compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the compositions and methods. The upper and lower limits of these smaller ranges may independently be included in the smaller ranges and are also encompassed within the compositions and methods, subject to any specifically excluded limit in the stated range. Where the stated range includes one or both of the limits, ranges excluding either or both of those included limits are also included in the compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the compositions and methods belong. Although any compositions and methods similar or equivalent to those described herein can also be used in the practice or testing of the compositions and methods, representative illustrative compositions and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present compositions and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the compositions and methods, which are, for clarity, described in the context of separate embodiments, may also be provided in combination in a single embodiment. Conversely, various features of the compositions and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present compositions and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Nucleic Acids, Polypeptides, Cells and Non-Human Animals

Aspects of the present disclosure include nucleic acids encoding kinase-modulated bioluminescent indicator (KiMBI) polypeptides. In certain embodiments, such nucleic acids encode a KiMBI polypeptide comprising a first bioluminescent enzyme fragment, a phospho-binding domain, a second bioluminescent enzyme fragment capable of forming an active bioluminescent enzyme with the first fragment via enzyme fragment complementation, and a kinase substrate which, when phosphorylated, is bound by the phospho-binding domain. When the kinase substrate is phosphorylated and bound by the phospho-binding domain, the second bioluminescent enzyme fragment adopts an unfavorable confirmation for enzyme fragment complementation with the first bioluminescent enzyme fragment. When the kinase substrate is not phosphorylated, the second bioluminescent enzyme fragment adopts a favorable confirmation for enzyme fragment complementation with the first bioluminescent enzyme fragment. KiMBI polypeptides encoded by any of the nucleic acids of the present disclosure are also provided.

The nucleic acids and polypeptides of the present disclosure find use in a variety of contexts, including real-time non-invasive visualization of kinase inhibition in vivo. Kinase indicators based on fluorescent proteins are widely used for live-cell experiments, where their high photon flux enables visualization of kinase activity with spatial and temporal resolutions on the micron and millisecond scales respectively. However, as the excitation light required for fluorescence imaging is difficult to deliver uniformly through tissue, fluorescent indicators can only be used in living mammals by inserting lenses of fibers to access specific sites of interest. This is invasive, severely restricts the field of view, and adds to experimental variability between animals. In contrast, the KiMBI polypeptides of the present disclosure enable an imaging procedure that is rapid and inexpensive, involving only injection of the bioluminescent enzyme substrate, anesthesia, and imaging. KiMBI imaging also allows each animal to provide information on the effects of one drug at multiple concentrations and timepoints. This efficiency is demonstrated in the Experimental section below, where although there was large individual variance in tumor sizes, which might introduce variability in kinase activity assays after dissection, the longitudinal nature of KiMBI imaging allowed assessment of the relative signal change within the same animal. In addition, KiMBI imaging allows the same group of subjects to report on multiple drugs, further improving efficiency. By reporting target inhibition in tissues of interest, KiMBI reporters will facilitate the acceleration of kinase inhibitor discovery and provide a link between kinase inhibition and, e.g., tumor responses. With KiMBI revealing whether a kinase inhibitor engages its target in vivo in a rapid and inexpensive manner, multiple candidates can be screened and their administered doses optimized. KiMBIs may also be employed to relate the extent and duration of kinase inhibition to, e.g., tumor shrinkage, enabling the establishment or refutation of proposed functions for a particular kinase in tumor growth.

In certain embodiments, the first bioluminescent enzyme fragment and the second bioluminescent enzyme fragment are fragments of a luciferase. In some instances, the luciferase is an ATP-independent luciferase. A non-limiting example of a luciferase is an Oplophorus gracilirostris luciferase or derivative thereof. By way of example, the luciferase may be a derivative of a Oplophorus gracilirostris luciferase, a non-limiting example of which is a NanoLuc (NLuc) luciferase. In some embodiments, a KiMBI polypeptide of the present disclosure comprises first and second complementary fragments of a luciferase predicted or known to have a beta-barrel structure, utilize coelenterazine, and not require ATP for catalysis, characteristics of Oplophorus gracilirostris luciferase. In some embodiments, a KiMBI polypeptide of the present disclosure comprises first and second complementary fragments of a luciferase selected from a Renilla luciferase (RLuc), a Renilla mutant luciferase (RLuc8), a Gaussia luciferase (GLuc), or a Oplophorus gracilirostris luciferase (OLuc). According to any of the embodiments herein, the first and second complementary fragments are of a luciferase other than a firefly luciferase (FLuc). Non-limiting examples of first bioluminescent enzyme fragments and second bioluminescent enzyme fragments capable of forming active bioluminescent enzymes via enzyme fragment complementation which may be implemented in a KiMBI polypeptide of the present disclosure include those provided in Table 1 below and variants thereof capable of forming active bioluminescent enzymes via enzyme fragment complementation.

A variety of phospho-binding domains may be implemented in a KiMBI polypeptide of the present disclosure. In some instances, the phospho-binding domain comprises a WW phospho-binding domain or a 14-3-3 phospho-binding domain, or phospho-binding variants thereof. When the phospho-binding domain comprises a WW phospho-binding domain, in certain embodiments, the WW phospho-binding domain is a Pin1 WW phospho-binding domain or phospho-binding variant thereof. Non-limiting examples of phospho-binding domains which may be implemented in a KiMBI polypeptide of the present disclosure include those provided in Table 1 below and phospho-binding domains variants thereof.

The kinase substrate implemented in a KiMBI polypeptide of the present disclosure is selected according to the kinase for which assessment of activity/inhibition via the KiMBI polypeptide is desired. Non-limiting examples of such kinases include a tyrosine kinase, external signal-regulated kinase (ERK), mitogen-activated protein kinase kinase (MEK), a cyclin-dependent kinase (CDK), an aurora kinase, a mitogen-activated protein kinase, ERBB1 (EGFR), ERBB2 (HER2), VEGFR, FGFR, Kit, PDGFR, AKT1/AKT2/AKT3, PIK3CA, BRAF, mTOR, SRC, ABL1, BCR-ABL, RET, AATK, EPHA2, EPHA5, CHK2, PKD1, ALK, BTK, MET, KIT, JAK, MEK1/2, Src, ROS1, ROCK, CDK4/CDK6, PI3Kδ, and LRRK2. The amino acid sequences of substrates for such kinases are known, and non-limiting examples of such substrates are provided in Table 1 below and variants thereof capable of serving as a substrate (e.g., comprising one or more phosphorylation sites) for the respective kinase. In some instances, the kinase substrate is a substrate for ERK. By way of example, the kinase substrate may be a Cdc25C ERK substrate.

In other embodiments, the kinase substrate may be a substrate for one, two or each of AKT1, AKT2, and AKT3. Akt is defined as representing Akt1, Akt2, Akt3, or any combination thereof. The designed Akt KiMBi will respond to inhibition of overall Akt activity levels, as the substrate sequence in Akt KiMBI, derived from BAD, is phosphorylated by Akt1, Akt2, and Akt3. It has been shown that Akt1 phosphorylates BAD in vitro, and also that either Akt2 or Akt3 depletion reduced BAD phosphorylation in glioma cells, demonstrating that the BAD substrate can be phosphorylated by any of the three Akt isoforms. If desired, a KiMBI polypeptide of the present disclosure may be specific for one Akt isoform, e.g., by addition of a domain that interacts with that isoform, e.g., by internal or terminal fusion.

The kinase substrate implemented in a KiMBI polypeptide may include any desired number of phosphorylation sites. According to some embodiments, the kinase substrate comprises a single phosphorylation site for the kinase of interest. In other embodiments, the kinase substrate comprises two or more phosphorylation sites for the kinase of interest, e.g., 2, 3, 4, 5 or more phosphorylation sites.

According to some embodiments, a KiMBI polypeptide of the present disclosure further comprises one or more domains that modulate the emission of the active bioluminescent enzyme, e.g., to shift the emission for enhanced tissue penetration in vivo as compared to the KiMBI polypeptide in the absence of the one or more modulator domains. In some instances, the KiMBI polypeptide comprises a modulator domain at the N-terminus, the C-terminus, or both. Modulator domains which may be implemented include, but are not limited to, one or more fluorescent protein domains, e.g., one or more fluorescent protein domains which exhibit resonance energy transfer (RET). Non-limiting examples of fluorescent protein domains include one or more fluorescent protein domains independently selected from a green fluorescent protein (GFP), a yellow fluorescent protein (YFP), a red fluorescent protein (RFP), and an orange fluorescent protein (OFP). When an RFP is employed, in some instances, the RFP is a tandem dimer Tomato (tdTomato) RFP. When an OFP is employed, in some instances, the OFP is CyOFP1. Non-limiting examples of modulator domains which may be implemented in a KiMBI polypeptide of the present disclosure include those provided in Table 1 below and variants thereof capable of modulating the emission of the active bioluminescent enzyme in the desired manner.

With the benefit of the present disclosure, it will be appreciated that the domains of the KiMBI polypeptides of the present disclosure may be provided in a variety of operable orders and orientations. For example, in some instances, a KiMBI polypeptide comprises, in order from N-terminal to C-terminal, or in order from C-terminal to N-terminal: the first bioluminescent enzyme fragment, the phospho-binding domain, the second bioluminescent enzyme fragment, and the kinase substrate. In certain embodiments, such a KiMBI polypeptide comprises a modulator domain (non-limiting examples of which include any of the modulator domains as described elsewhere herein) at the N-terminus, the C-terminus, or both. In other instances, a KiMBI polypeptide comprises, in order from N-terminal to C-terminal, or in order from C-terminal to N-terminal: the first bioluminescent enzyme fragment, the phospho-binding domain, the kinase substrate, and the second bioluminescent enzyme fragment. In certain embodiments, such a KiMBI polypeptide comprises a modulator domain (non-limiting examples of which include any of the modulator domains as described elsewhere herein) at the N-terminus, the C-terminus, or both.

In certain embodiments, the various domains of a KiMBI polypeptide are fused directly to each other. In other embodiments, a KiMBI polypeptide comprises one or more linkers, where a linker links two domains of the KiMBI polypeptide. When one or more linkers are employed, the lengths and amino acid content of the one or more linkers may be independently selected to enable or enhance the functionality of the KiMBI polypeptide, e.g., by providing useful spacing and/or flexibility between domains of the KiMBI polypeptide, such as between the first and second bioluminescent enzyme fragments (permitting or enhancing enzyme fragment complementation), between the phospho-binding and a phosphorylated kinase substrate, between one or more modulator domains and an active bioluminescent enzyme, and/or the like. As will be appreciated with the benefit of the present disclosure, useful lengths and/or amino acid contents of one or more linkers in a KiMBI polypeptide will vary depending upon the particular enzyme fragments, phospho-binding domain, and kinase substrate employed. Non-limiting examples of linkers that may be employed in a KiMBI polypeptide are provided in Table 1 below. In some instances, a linker is a flexible linker. A flexible linker may be a GlySer (GS) linker, a non-limiting example of which comprises or consists of the sequence GGSGGGGSGGGGSGGGGSGGGGSGGGGSGG (SEQ ID NO:61).

The nucleotide and amino acid sequences of exemplary KiMBI constructs and domains that find use for incorporating into KiMBIs are provided in Table 1 below. For each KiMBI sequence, the domains as ordered from N- to C-terminus are listed in the left column. The sequence in the middle column indicates the domains by alternating underlining.

TABLE 1
Nucleotide and Amino Acid Sequences

| | SEQUENCE | SEQ ID NO |
|---|---|---|
| Blue KiMBI | ATGGTGAGCAAGGGCGAGGAGCTGATCAAGGAGAACATGA | 1 |
| Nucleotide | GAAGCAAGCTGTACCTGGAAGGCAGCGTGAACGGCCACCA | |
| Sequence | GTTCAAGTGCACCCACGAAGGGGAGGGCAAGCCCTACGAG | |
| •CyOFP(ΔC7) | GGCAAGCAGACCAACAGGATCAAGGTGGTGGAGGGAGGCC | |
| •Linker | CCCTGCCGTTCGCATTCGACATCCTGGCCACCCACTTTATGT | |
| •LgBIT(ΔN2) | ACGGGAGCAAGGTGTTCATCAAGTACCCCGCCGACCTCCCC | |
| •Linker | GATTATTTTAAGCAGTCCTTCCCTGAGGGCTTCACATGGGAG | |
| •WW(F25A) | AGAGTCATGGTGTTCGAAGACGGGGGCGTGCTGACCGCCA | |
| •SmBIT(−C3) | CCCAGGACACCAGCCTCCAGGACGGCGAGCTCATCTACAAC | |
| •ERKsub | GTCAAGGTCAGAGGGGTGAACTTCCCAGCCAACGGCCCCG | |
| | TGATGCAGAAGAAAACACTGGGCTGGGAGCCCAGCACCGA | |
| | GACCATGTACCCCGCTGACGGCGGCCTGGAAGGCAGATGC | |
| | GACAAGGCCCTGAAGCTCGTGGGGGGGGGCCACCTGCACG | |
| | TCAACTTCAAGACCACATACAAGTCCAAGAAACCCGTGAAGA | |
| | TGCCCGGCGTCCACTACGTGGACCGCAGACTGGAAAGAATC | |
| | AAGGAGGCCGACAACGAGACCTACGTCGAGCAGTACGAGC | |
| | ACGCTGTGGCCAGATACTCCAACCTGGGCGGAGGCTTCACA | |
| | CTCGAAGATTTCGTTGGGGACTGGGAACAGACAGCCGCCTA | |
| | CAACCTGGACCAAGTCCTTGAACAGGGAGGTGTGTCCAGTT | |
| | TGCTGCAGAATCTCGCCGTGTCCGTAACTCCGATCCAAAGG | |
| | ATTGTCCGGAGCGGTGAAAATGCCCTGAAGATCGACATCCA | |
| | TGTCATCATCCCGTATGAAGGTCTGAGCGCCGACCAAATGG | |
| | CCCAGATCGAAGAGGTGTTTAAGGTGGTGTACCCTGTGGAT | |
| | GATCATCACTTTAAGGTGATCCTGCCCTATGGCACACTGGTA | |
| | ATCGACGGGGTTACGCCGAACATGCTGAACTATTTCGGACG | |
| | GCCGTATGAAGGCATCGCCGTGTTCGACGGCAAAAAGATCA | |
| | CTGTAACAGGGACCCTGTGGAACGGCAACAAAATTATCGAC | |
| | GAGCGCCTGATCACCCCCGACGGCTCCATGCTGTTCCGAGT | |
| | AACCATCAACAGCATCATGGCGGACGAGGAGAAGCTGCCG | |
| | CCCGGCTGGGAGAAGCGCATGAGCCGCAGCTCAGGCCGAG | |
| | TGTACTACGCCAACCACATCACTAACGCCAGCCAGTGGGAG | |
| | CGGCCCAGCGGCAACAGCAGCAGTGGTGGCAAAAACGGGC | |
| | AGGGGGAGCCTGCCAGGGTGACCGGCTACCGGCTGTTCGA | |
| | GCCAGATAGCCCTAGAACTCCAGTGGATAAAGCAAAGCTGT | |
| | CATTCCAATTCCCC | |
| Blue KiMBI Amino | MVSKGEELIKENMRSKLYLEGSVNGHQFKCTHEGEGKPYEGK | 2 |
| Acid Sequence | QTNRIKVVEGGPLPFAFDILATHFMYGSKVFIKYPADLPDYFKQ | |
| •CyOFP(ΔC7) | SFPEGFTWERVMVFEDGGVLTATQDTSLQDGELIYNVKVRGV | |
| •Linker | NFPANGPVMQKKTLGWEPSTETMYPADGGLEGRCDKALKLVG | |
| •LgBiT(ΔN2) | GGHLHVNFKTTYKSKKPVKMPGVHYVDRRLERIKEADNETYVE | |
| •Linker | QYEHAVARYSNLGGGFTLEDFVGDWEQTAAYNLDQVLEQGG | |
| •WW(F25A) | VSSLLQNLAVSVTPIQRIVRSGENALKIDIHVIIPYEGLSADQMAQ | |
| •SmBIT(ΔC3) | IEEVFKVVYPVDDHHFKVILPYGTLVIDGVTPNMLNYFGRPYEGI | |
| •ERKsub | AVFDGKKITVTGTLWNGNKIIDERLITPDGSMLFRVTINSIMADE | |
| | EKLPPGWEKRMSRSSGRVYYANHITNASQWERPSGNSSSGG | |
| | KNGQGEPARVTGYRLFEPDSPRTPVDKAKLSFQFP | |
| tdTomato KiMBI | ATGGTGAGCAAGGGCGAGGAGGTCATCAAAGAGTTCATGCG | 3 |
| Nucleotide | CTTCAAGGTGCGCATGGAGGGCTCCATGAACGGCCACGAG | |
| Sequence | TTCGAGATCGAGGGCGAGGGCGAGGGCCGCCCCTACGAGG | |
| •tdTomato(ΔC9) | GCACCCAGACCGCCAAGCTGAAGGTGACCAAGGGCGGCCC | |
| •Linker | CCTGCCCTTCGCCTGGGACATCCTGTCCCCCCAGTTCATGT | |
| •LgBIT(ΔN2) | ACGGCTCCAAGGCGTACGTGAAGCACCCCGCCGACATCCC | |
| •Linker | CGATTACAAGAAGCTGTCCTTCCCCGAGGGCTTCAAGTGGG | |
| •WW | AGCGCGTGATGAACTTCGAGGACGGCGGTCTGGTGACCGT | |
| •SmBIT(ΔC3) | GACCCAGGACTCCTCCCTGCAGGACGGCACGCTGATCTACA | |
| •ERKsub | AGGTGAAGATGCGCGGCACCAACTTCCCCCCCGACGGCCC | |
| | CGTAATGCAGAAGAAGACCATGGGCTGGGAGGCCTCCACC | |
| | GAGCGCCTGTACCCCCGCGACGGCGTGCTGAAGGGCGAGA | |
| | TCCACCAGGCCCTGAAGCTGAAGGACGGCGGCCACTACCT | |
| | GGTGGAGTTCAAGACCATCTACATGGCCAAGAAGCCCGTGC | |
| | AACTGCCCGGCTACTACTACGTGGACACCAAGCTGGACATC | |
| | ACCTCCCACAACGAGGACTACACCATCGTGGAACAGTACGA | |
| | GCGCTCCGAGGGCCGCCACCACCTGTTCCTGGGGCATGGC | |
| | ACCGGCAGCACCGGCAGCGGCAGCTCCGGCACCGCCTCCT | |
| | CCGAGGACAACAACATGGCCGTCATCAAAGAGTTCATGCGC | |
| | TTCAAGGTGCGCATGGAGGGCTCCATGAACGGCCACGAGTT | |
| | CGAGATCGAGGGCGAGGGCGAGGGCCGCCCCTACGAGGG | |
| | CACCCAGACCGCCAAGCTGAAGGTGACCAAGGGCGGCCCC | |
| | CTGCCCTTCGCCTGGGACATCCTGTCCCCCCAGTTCATGTA | |
| | CGGCTCCAAGGCGTACGTGAAGCACCCCGCCGACATCCCC | |
| | GATTACAAGAAGCTGTCCTTCCCCGAGGGCTTCAAGTGGGA | |
| | GCGCGTGATGAACTTCGAGGACGGCGGTCTGGTGACCGTG | |
| | ACCCAGGACTCCTCCCTGCAGGACGGCACGCTGATCTACAA | |
| | GGTGAAGATGCGCGGCACCAACTTCCCCCCCGACGGCCCC | |
| | GTAATGCAGAAGAAGACCATGGGCTGGGAGGCCTCCACCG | |
| | AGCGCCTGTACCCCCGCGACGGCGTGCTGAAGGGCGAGAT | |
| | CCACCAGGCCCTGAAGCTGAAGGACGGCGGCCACTACCTG | |
| | GTGGAGTTCAAGACCATCTACATGGCCAAGAAGCCCGTGCA | |
| | ACTGCCCGGCTACTACTACGTGGACACCAAGCTGGACATCA | |
| | CCTCCCACAACGAGGACTACACCATCGTGGAACAGTACGAG | |
| | CGCTCCGAGGGCCGCCACCACCTGTTCCGGCTGGAAGATTT | |
| | CGTTGGGGACTGGGAACAGACAGCCGCCTACAACCTGGAC | |
| | CAAGTCCTTGAACAGGGAGGTGTGTCCAGTTTGCTGCAGAA | |
| | TCTCGCCGTGTCCGTAACTCCGATCCAAAGGATTGTCCGGA | |
| | GCGGTGAAAATGCCCTGAAGATCGACATCCATGTCATCATC | |
| | CCGTATGAAGGTCTGAGCGCCGACCAAATGGCCCAGATCGA | |
| | AGAGGTGTTTAAGGTGGTGTACCCTGTGGATGATCATCACTT | |
| | TAAGGTGATCCTGCCCTATGGCACACTGGTAATCGACGGGG | |
| | TTACGCCGAACATGCTGAACTATTTCGGACGGCCGTATGAA | |
| | GGCATCGCCGTGTTCGACGGCAAAAAGATCACTGTAACAGG | |
| | GACCCTGTGGAACGGCAACAAAATTATCGACGAGCGCCTGA | |
| | TCACCCCCGACGGCTCCATGCTGTTCCGAGTAACCATCAAC | |
| | AGCATCATGGCGGACGAGGAGAAGCTGCCGCCCGGCTGGG | |
| | AGAAGCGCATGAGCCGCAGCTCAGGCCGAGTGTACTACTTC | |
| | AACCACATCACTAACGCCAGCCAGTGGGAGCGGCCCAGCG | |
| | GCAACAGCAGCAGTGGTGGCAAAAACGGGCAGGGGGAGCC | |
| | TGCCAGGGTGACCGGCTACCGGCTGTTCGAGCCAGATAGC | |
| | CCTAGAACTCCAGTGGATAAAGCAAAGCTGTCATTCCAATTC | |
| | CCC | |
| tdTomato KiMBI | MVSKGEEVIKEFMRFKVRMEGSMNGHEFEIEGEGEGRPYEGT | 4 |
| Amino Acid | QTAKLKVTKGGPLPFAWDILSPQFMYGSKAYVKHPADIPDYKK | |
| Sequence | LSFPEGFKWERVMNFEDGGLVTVTQDSSLQDGTLIYKVKMRG | |
| •tdTomato(ΔC9) | TNFPPDGPVMQKKTMGWEASTERLYPRDGVLKGEIHQALKLK | |
| •Linker | DGGHYLVEFKTIYMAKKPVQLPGYYYVDTKLDITSHNEDYTIVE | |
| •LgBIT(ΔN2) | QYERSEGRHHLFLGHGTGSTGSGSSGTASSEDNNMAVIKEFM | |
| •Linker | RFKVRMEGSMNGHEFEIEGEGEGRPYEGTQTAKLKVTKGGPL | |
| •WW | PFAWDILSPQFMYGSKAYVKHPADIPDYKKLSFPEGFKWERVM | |
| •SmBIT(ΔC3) | NFEDGGLVTVTQDSSLQDGTLIYKVKMRGTNFPPDGPVMQKK | |
| •ERKsub | TMGWEASTERLYPRDGVLKGEIHQALKLKDGGHYLVEFKTIYM | |
| | AKKPVQLPGYYYVDTKLDITSHNEDYTIVEQYERSEGRHHLFRL | |
| | EDFVGDWEQTAAYNLDQVLEQGGVSSLLQNLAVSVTPIQRIVR | |
| | SGENALKIDIHVIIPYEGLSADQMAQIEEVFKVVYPVDDHHFKVIL | |
| | PYGTLVIDGVTPNMLNYFGRPYEGIAVFDGKKITVTGTLWNGNK | |
| | IIDERLITPDGSMLFRVTINSIMADEEKLPPGWEKRMSRSSGRV | |
| | YYFNHITNASQWERPSGNSSSGGKNGQGEPARVTGYRLFEPD | |
| | SPRTPVDKAKLSFQFP | |
| Orange KiMBI | ATGGTGAGCAAGGGCGAGGAGCTGATCAAGGAGAACATGA | 5 |
| Nucleotide | GAAGCAAGCTGTACCTGGAAGGCAGCGTGAACGGCCACCA | |
| Sequence | GTTCAAGTGCACCCACGAAGGGGAGGGCAAGCCCTACGAG | |
| •CyOFP(ΔC7) | GGCAAGCAGACCAACAGGATCAAGGTGGTGGAGGGAGGCC | |
| •Linker | CCCTGCCGTTCGCATTCGACATCCTGGCCACCCACTTTATGT | |
| •LgBiT(ΔN2) | ACGGGAGCAAGGTGTTCATCAAGTACCCCGCCGACCTCCCC | |
| •Linker | GATTATTTTAAGCAGTCCTTCCCTGAGGGCTTCACATGGGAG | |
| •WW | AGAGTCATGGTGTTCGAAGACGGGGGCGTGCTGACCGCCA | |
| •SmBIT(ΔC3, | CCCAGGACACCAGCCTCCAGGACGGCGAGCTCATCTACAAC | |
| L163E) | GTCAAGGTCAGAGGGGTGAACTTCCCAGCCAACGGCCCCG | |
| •ERKsub | TGATGCAGAAGAAAACACTGGGCTGGGAGCCCAGCACCGA | |
| •Linker | GACCATGTACCCCGCTGACGGCGGCCTGGAAGGCAGATGC | |
| •CyOFP(ΔN6) | GACAAGGCCCTGAAGCTCGTGGGGGGGGGCCACCTGCACG | |
| | TCAACTTCAAGACCACATACAAGTCCAAGAAACCCGTGAAGA | |
| | TGCCCGGCGTCCACTACGTGGACCGCAGACTGGAAAGAATC | |
| | AAGGAGGCCGACAACGAGACCTACGTCGAGCAGTACGAGC | |
| | ACGCTGTGGCCAGATACTCCAACCTGGGCGGAGGCTTCACA | |
| | CTCGAAGATTTCGTTGGGGACTGGGAACAGACAGCCGCCTA | |
| | CAACCTGGACCAAGTCCTTGAACAGGGAGGTGTGTCCAGTT | |
| | TGCTGCAGAATCTCGCCGTGTCCGTAACTCCGATCCAAAGG | |
| | ATTGTCCGGAGCGGTGAAAATGCCCTGAAGATCGACATCCA | |
| | TGTCATCATCCCGTATGAAGGTCTGAGCGCCGACCAAATGG | |
| | CCCAGATCGAAGAGGTGTTTAAGGTGGTGTACCCTGTGGAT | |
| | GATCATCACTTTAAGGTGATCCTGCCCTATGGCACACTGGTA | |
| | ATCGACGGGGTTACGCCGAACATGCTGAACTATTTCGGACG | |
| | GCCGTATGAAGGCATCGCCGTGTTCGACGGCAAAAAGATCA | |
| | CTGTAACAGGGACCCTGTGGAACGGCAACAAAATTATCGAC | |
| | GAGCGCCTGATCACCCCCGACGGCTCCATGCTGTTCCGAGT | |
| | AACCATCAACAGCATCATGGCGGACGAGGAGAAGCTGCCG | |
| | CCCGGCTGGGAGAAGCGCATGAGCCGCAGCTCAGGCCGAG | |
| | TGTACTACTTCAACCACATCACTAACGCCAGCCAGTGGGAG | |
| | CGGCCCAGCGGCAACAGCAGCAGTGGTGGCAAAAACGGGC | |
| | AGGGGGAGCCTGCCAGGGTGACCGGCTACCGGGAGTTCGA | |
| | GCCAGATAGCCCTAGAACTCCAGTGGATAAAGCAAAGCTGT | |
| | CATTCCAATTCCCCAGGCACGAACTCATTAAAGAAAATATGC | |
| | GGTCCAAGCTCTATCTCGAGGGAAGTGTGAATGGTCATCAA | |
| | TTTAAGTGTACTCATGAGGGCGAAGGTAAACCTTACGAAGG | |
| | AAAGCAAACGAATCGAATAAAGGTCGTGGAAGGCGGACCGC | |
| | TCCCTTTTGCCTTCGATATTCTGGCTACACATTTCATGTATGG | |
| | ATCTAAAGTCTTTATTAAGTATCCTGCGGATCTGCCAGACTA | |
| | CTTTAAGCAGAGCTTTCCAGAAGGATTTACTTGGGAACGAGT | |
| | GATGGTCTTTGAGGATGGAGGTGTCCTCACAGCTACGCAAG | |
| | ATACTTCCCTTCAAGATGGAGAACTGATTTACAACGTGAAAG | |
| | TGCGCGGTGTCAATTTTCCTGCAAATGGTCCAGTCATGCAAA | |
| | AGAAGACCCTCGGATGGGAACCTTCTACAGAAACAATGTAT | |
| | CCTGCAGATGGAGGTCTCGAGGGTCGGTGTGATAAGGCTCT | |
| | TAAACTGGTCGGAGGAGGTCATCTCCATGTGAATTTCAAAAC | |
| | TACCTATAAAAGCAAAAAGCCTGTCAAAATGCCAGGTGTACA | |
| | TTATGTCGATCGACGGCTCGAGCGGATAAAGGAAGCTGATA | |
| | ATGAAACGTATGTGGAACAGTATGAACATGCCGTCGCACGG | |
| | TATAGTAATCTCGGTGGCGGAATGGATGAACTCTATAAG | |
| Orange KiMBI | MVSKGEELIKENMRSKLYLEGSVNGHQFKCTHEGEGKPYEGK | 6 |
| Amino Acid | QTNRIKVVEGGPLPFAFDILATHFMYGSKVFIKYPADLPDYFKQ | |
| Sequence | SFPEGFTWERVMVFEDGGVLTATQDTSLQDGELIYNVKVRGV | |
| •CyOFP(ΔC7) | NFPANGPVMQKKTLGWEPSTETMYPADGGLEGRCDKALKLVG | |
| •Linker | GGHLHVNFKTTYKSKKPVKMPGVHYVDRRLERIKEADNETYVE | |
| •LgBIT(ΔN2) | QYEHAVARYSNLGGGFTLEDFVGDWEQTAAYNLDQVLEQGG | |
| •Linker | VSSLLQNLAVSVTPIQRIVRSGENALKIDIHVIIPYEGLSADQMAQ | |
| •WW | IEEVFKVVYPVDDHHFKVILPYGTLVIDGVTPNMLNYFGRPYEGI | |
| •SmBIT(ΔC3, | AVFDGKKITVTGTLWNGNKIIDERLITPDGSMLFRVTINSIMADE | |
| L163E) | EKLPPGWEKRMSRSSGRVYYFNHITNASQWERPSGNSSSGG | |
| •ERKsub | KNGQGEPARVTGYREFEPDSPRTPVDKAKLSFQFPRHELIKEN | |
| •Linker | MRSKLYLEGSVNGHQFKCTHEGEGKPYEGKQTNRIKVVEGGP | |
| •CyOFP(−N6) | LPFAFDILATHFMYGSKVFIKYPADLPDYFKQSFPEGFTWERVM | |
| | VFEDGGVLTATQDTSLQDGELIYNVKVRGVNFPANGPVMQKKT | |
| | LGWEPSTETMYPADGGLEGRCDKALKLVGGGHLHVNFKTTYK | |
| | SKKPVKMPGVHYVDRRLERIKEADNETYVEQYEHAVARYSNLG | |
| | GGMDELYK | |
| Orange | ATGGTGAGCAAGGGCGAGGAGCTGATCAAGGAGAACATGA | 7 |
| oKiMBI_Akt | GAAGCAAGCTGTACCTGGAAGGCAGCGTGAACGGCCACCA | |
| Nucleotide | GTTCAAGTGCACCCACGAAGGGGAGGGCAAGCCCTACGAG | |
| Sequence | GGCAAGCAGACCAACAGGATCAAGGTGGTGGAGGGAGGCC | |
| •CyOFP1(ΔC7) | CCCTGCCGTTCGCATTCGACATCCTGGCCACCCACTTTATGT | |
| •LgBiT | ACGGGAGCAAGGTGTTCATCAAGTACCCCGCCGACCTCCCC | |
| •linker | GATTATTTTAAGCAGTCCTTCCCTGAGGGCTTCACATGGGAG | |
| •14-3-3 | AGAGTCATGGTGTTCGAAGACGGGGGCGTGCTGACCGCCA | |
| •linker | CCCAGGACACCAGCCTCCAGGACGGCGAGCTCATCTACAAC | |
| •SmBIT | GTCAAGGTCAGAGGGGTGAACTTCCCAGCCAACGGCCCCG | |
| •linker | TGATGCAGAAGAAAACACTGGGCTGGGAGCCCAGCACCGA | |
| •BAD substrate | GACCATGTACCCCGCTGACGGCGGCCTGGAAGGCAGATGC | |
| •linker | GACAAGGCCCTGAAGCTCGTGGGGGGGGGCCACCTGCACG | |
| •CyOFP1(ΔN6) | TCAACTTCAAGACCACATACAAGTCCAAGAAACCCGTGAAGA | |
| | TGCCCGGCGTCCACTACGTGGACCGCAGACTGGAAAGAATC | |
| | AAGGAGGCCGACAACGAGACCTACGTCGAGCAGTACGAGC | |
| | ACGCTGTGGCCAGATACTCCAACCTGGGCGGAGGCTTCACA | |
| | CTCGAAGATTTCGTTGGGGACTGGGAACAGACAGCCGCCTA | |
| | CAACCTGGACCAAGTCCTTGAACAGGGAGGTGTGTCCAGTT | |
| | TGCTGCAGAATCTCGCCGTGTCCGTAACTCCGATCCAAAGG | |
| | ATTGTCCGGAGCGGTGAAAATGCCCTGAAGATCGACATCCA | |
| | TGTCATCATCCCGTATGAAGGTCTGAGCGCCGACCAAATGG | |
| | CCCAGATCGAAGAGGTGTTTAAGGTGGTGTACCCTGTGGAT | |
| | GATCATCACTTTAAGGTGATCCTGCCCTATGGCACACTGGTA | |
| | ATCGACGGGGTTACGCCGAACATGCTGAACTATTTCGGACG | |
| | GCCGTATGAAGGCATCGCCGTGTTCGACGGCAAAAAGATCA | |
| | CTGTAACAGGGACCCTGTGGAACGGCAACAAAATTATCGAC | |
| | GAGCGCCTGATCACCCCCGACGGCTCCATGCTGTTCCGAGT | |
| | AACCATCAACAGCGGCGGAGGTAGTATGGATAAAAATGAGC | |
| | TGGTTCAGAAGGCCAAACTGGCCGAGCAGGCTGAGCGATAT | |
| | GATGACATGGCAGCCTGCATGAAGTCTGTAACTGAGCAAGG | |
| | AGCTGAATTATCCAATGAGGAGAGGAATCTTCTCTCAGTTGC | |
| | TTATAAAAATGTTGTAGGAGCCCGTAGGTCATCTTGGAGGGT | |
| | CGTCTCAAGTATTGAACAAAAGACGGAAGGTGCTGAGAAAA | |
| | AACAGCAGATGGCTCGAGAATACAGAGAGAAAATTGAGACG | |
| | GAGCTAAGAGATATCTGCAATGATGTACTGTCTCTTTTGGAA | |
| | AAGTTCTTGATCCCCAATGCTTCACAAGCAGAGAGCAAAGTC | |
| | TTCTATTTGAAAATGAAAGGAGATTACTACCGTTACTTGGCT | |
| | GAGGTTGCCGCTGGTGATGACAAGAAAGGGATTGTCGATCA | |
| | GTCACAACAAGCATACCAAGAAGCTTTTGAAATCAGCAAAAA | |
| | GGAAATGCAACCAACACATCCTATCAGACTGGGTCTGGCCC | |
| | TTAACTTCTCTGTGTTCTATTATGAGATTCTGAACTCCCCAGA | |
| | GAAAGCCTGCTCTCTTGCAAAGACAGCTTTTGATGAAGCCAT | |
| | TGCTGAACTTGATACATTAAGTGAAGAGTCATACAAAGACAG | |
| | CACGCTAATAATGCAATTACTGAGAGACAACTTGACATTGTG | |
| | GACATCGGATACCGGAGGGAGTGGAGGTGGAGGCTCTGGA | |
| | GGAGGGGGTTCAGGTGGTGGAGGCAGCGGCGGAGGTGGA | |
| | TCCGGAGGAGGTGGCAGTGGGGGAGTGACCGGCTACCGG | |
| | CTGTTCGAGCCATTTCGAGGCCGTTCTAGAACCGCTCCGGC | |
| | TAACCTCTGGGCAAGGCACGAACTCATTAAAGAAAATATGCG | |
| | GTCCAAGCTCTATCTCGAGGGAAGTGTGAATGGTCATCAATT | |
| | TAAGTGTACTCATGAGGGCGAAGGTAAACCTTACGAAGGAA | |
| | AGCAAACGAATCGAATAAAGGTCGTGGAAGGCGGACCGCTC | |
| | CCTTTTGCCTTCGATATTCTGGCTACACATTTCATGTATGGAT | |
| | CTAAAGTCTTTATTAAGTATCCTGCGGATCTGCCAGACTACT | |
| | TTAAGCAGAGCTTTCCAGAAGGATTTACTTGGGAACGAGTGA | |
| | TGGTCTTTGAGGATGGAGGTGTCCTCACAGCTACGCAAGAT | |
| | ACTTCCCTTCAAGATGGAGAACTGATTTACAACGTGAAAGTG | |
| | CGCGGTGTCAATTTTCCTGCAAATGGTCCAGTCATGCAAAAG | |
| | AAGACCCTCGGATGGGAACCTTCTACAGAAACAATGTATCCT | |
| | GCAGATGGAGGTCTCGAGGGTCGGTGTGATAAGGCTCTTAA | |
| | ACTGGTCGGAGGAGGTCATCTCCATGTGAATTTCAAAACTAC | |
| | CTATAAAAGCAAAAAGCCTGTCAAAATGCCAGGTGTACATTA | |
| | TGTCGATCGACGGCTCGAGCGGATAAAGGAAGCTGATAATG | |
| | AAACGTATGTGGAACAGTATGAACATGCCGTCGCACGGTAT | |
| | AGTAATCTCGGTGGCGGAATGGATGAACTCTATAAG | |
| Orange | MVSKGEELIKENMRSKLYLEGSVNGHQFKCTHEGEGKPYEGK | 8 |
| oKiMBI_Akt Amino | QTNRIKVVEGGPLPFAFDILATHFMYGSKVFIKYPADLPDYFKQ | |
| Acid Sequence | SFPEGFTWERVMVFEDGGVLTATQDTSLQDGELIYNVKVRGV | |
| •CyOFP1(ΔC7) | NFPANGPVMQKKTLGWEPSTETMYPADGGLEGRCDKALKLVG | |
| •LgBiT | GGHLHVNFKTTYKSKKPVKMPGVHYVDRRLERIKEADNETYVE | |
| •linker | QYEHAVARYSNLGGGFTLEDFVGDWEQTAAYNLDQVLEQGG | |
| •14-3-3 | VSSLLQNLAVSVTPIQRIVRSGENALKIDIHVIIPYEGLSADQMAQ | |
| •linker | IEEVFKVVYPVDDHHFKVILPYGTLVIDGVTPNMLNYFGRPYEGI | |
| •SmBIT | AVFDGKKITVTGTLWNGNKIIDERLITPDGSMLFRVTINSGGGS | |
| •linker | MDKNELVQKAKLAEQAERYDDMAACMKSVTEQGAELSNEERN | |
| •BAD substrate | LLSVAYKNVVGARRSSWRVVSSIEQKTEGAEKKQQMAREYRE | |
| •linker | KIETELRDICNDVLSLLEKFLIPNASQAESKVFYLKMKGDYYRYL | |
| •CyOFP1(ΔN6) | AEVAAGDDKKGIVDQSQQAYQEAFEISKKEMQPTHPIRLGLAL | |
| | NFSVFYYEILNSPEKACSLAKTAFDEAIAELDTLSEESYKDSTLI | |
| | MQLLRDNLTLWTSDTGGSGGGGSGGGGSGGGGSGGGGSGG | |
| | GGSGGVTGYRLFEPFRGRSRTAPANLWARHELIKENMRSKLY | |
| | LEGSVNGHQFKCTHEGEGKPYEGKQTNRIKVVEGGPLPFAFDI | |
| | LATHFMYGSKVFIKYPADLPDYFKQSFPEGFTWERVMVFEDGG | |
| | VLTATQDTSLQDGELIYNVKVRGVNFPANGPVMQKKTLGWEP | |
| | STETMYPADGGLEGRCDKALKLVGGGHLHVNFKTTYKSKKPV | |
| | KMPGVHYVDRRLERIKEADNETYVEQYEHAVARYSNLGGGMD | |
| | ELYK | |
| Blue bKiMBI_Akt | ATGGTGAGCAAGGGCGAGGAGCTGATCAAGGAGAACATGA | 9 |
| Nucleotide | GAAGCAAGCTGTACCTGGAAGGCAGCGTGAACGGCCACCA | |
| Sequence | GTTCAAGTGCACCCACGAAGGGGAGGGCAAGCCCTACGAG | |
| •CyOFP1(ΔC7) | GGCAAGCAGACCAACAGGATCAAGGTGGTGGAGGGAGGCC | |
| •LgBiT | CCCTGCCGTTCGCATTCGACATCCTGGCCACCCACTTTATGT | |
| •linker | ACGGGAGCAAGGTGTTCATCAAGTACCCCGCCGACCTCCCC | |
| •14-3-3 | GATTATTTTAAGCAGTCCTTCCCTGAGGGCTTCACATGGGAG | |
| •linker | AGAGTCATGGTGTTCGAAGACGGGGGCGTGCTGACCGCCA | |
| •SmBIT | CCCAGGACACCAGCCTCCAGGACGGCGAGCTCATCTACAAC | |
| •BAD substrate | GTCAAGGTCAGAGGGGTGAACTTCCCAGCCAACGGCCCCG | |
| | TGATGCAGAAGAAAACACTGGGCTGGGAGCCCAGCACCGA | |
| | GACCATGTACCCCGCTGACGGCGGCCTGGAAGGCAGATGC | |
| | GACAAGGCCCTGAAGCTCGTGGGGGGGGGCCACCTGCACG | |
| | TCAACTTCAAGACCACATACAAGTCCAAGAAACCCGTGAAGA | |
| | TGCCCGGCGTCCACTACGTGGACCGCAGACTGGAAAGAATC | |
| | AAGGAGGCCGACAACGAGACCTACGTCGAGCAGTACGAGC | |
| | ACGCTGTGGCCAGATACTCCAACCTGGGCGGAGGCTTCACA | |
| | CTCGAAGATTTCGTTGGGGACTGGGAACAGACAGCCGCCTA | |
| | CAACCTGGACCAAGTCCTTGAACAGGGAGGTGTGTCCAGTT | |
| | TGCTGCAGAATCTCGCCGTGTCCGTAACTCCGATCCAAAGG | |
| | ATTGTCCGGAGCGGTGAAAATGCCCTGAAGATCGACATCCA | |
| | TGTCATCATCCCGTATGAAGGTCTGAGCGCCGACCAAATGG | |
| | CCCAGATCGAAGAGGTGTTTAAGGTGGTGTACCCTGTGGAT | |
| | GATCATCACTTTAAGGTGATCCTGCCCTATGGCACACTGGTA | |
| | ATCGACGGGGTTACGCCGAACATGCTGAACTATTTCGGACG | |
| | GCCGTATGAAGGCATCGCCGTGTTCGACGGCAAAAAGATCA | |
| | CTGTAACAGGGACCCTGTGGAACGGCAACAAAATTATCGAC | |
| | GAGCGCCTGATCACCCCCGACGGCTCCATGCTGTTCCGAGT | |
| | AACCATCAACAGCGGCGGAGGTAGTATGGATAAAAATGAGC | |
| | TGGTTCAGAAGGCCAAACTGGCCGAGCAGGCTGAGCGATAT | |
| | GATGACATGGCAGCCTGCATGAAGTCTGTAACTGAGCAAGG | |
| | AGCTGAATTATCCAATGAGGAGAGGAATCTTCTCTCAGTTGC | |
| | TTATAAAAATGTTGTAGGAGCCCGTAGGTCATCTTGGAGGGT | |
| | CGTCTCAAGTATTGAACAAAAGACGGAAGGTGCTGAGAAAA | |
| | AACAGCAGATGGCTCGAGAATACAGAGAGAAAATTGAGACG | |
| | GAGCTAAGAGATATCTGCAATGATGTACTGTCTCTTTTGGAA | |
| | AAGTTCTTGATCCCCAATGCTTCACAAGCAGAGAGCAAAGTC | |
| | TTCTATTTGAAAATGAAAGGAGATTACTACCGTTACTTGGCT | |
| | GAGGTTGCCGCTGGTGATGACAAGAAAGGGATTGTCGATCA | |
| | GTCACAACAAGCATACCAAGAAGCTTTTGAAATCAGCAAAAA | |
| | GGAAATGCAACCAACACATCCTATCAGACTGGGTCTGGCCC | |
| | TTAACTTCTCTGTGTTCTATTATGAGATTCTGAACTCCCCAGA | |
| | GAAAGCCTGCTCTCTTGCAAAGACAGCTTTTGATGAAGCCAT | |
| | TGCTGAACTTGATACATTAAGTGAAGAGTCATACAAAGACAG | |
| | CACGCTAATAATGCAATTACTGAGAGACAACTTGACATTGTG | |
| | GACATCGGATACCGGAGGGAGTGGAGGTGGAGGCTCTGGA | |
| | GGAGGGGGTTCAGGTGGTGGAGGCAGCGGCGGAGGTGGA | |
| | TCCGGAGGAGGTGGCAGTGGGGGAGTGACCGGCTACCGG | |
| | CTGTTCGAGCCATTTCGAGGCCGTTCTAGAAGTGCTCCGCC | |
| | TAACCTCTGGGCA | |
| Blue bKiMBI_Akt | MVSKGEELIKENMRSKLYLEGSVNGHQFKCTHEGEGKPYEGK | 10 |
| Amino Acid | QTNRIKVVEGGPLPFAFDILATHFMYGSKVFIKYPADLPDYFKQ | |
| Sequence | SFPEGFTWERVMVFEDGGVLTATQDTSLQDGELIYNVKVRGV | |
| •CyOFP1(ΔC7) | NFPANGPVMQKKTLGWEPSTETMYPADGGLEGRCDKALKLVG | |
| •LgBiT | GGHLHVNFKTTYKSKKPVKMPGVHYVDRRLERIKEADNETYVE | |
| •linker | QYEHAVARYSNLGGGFTLEDFVGDWEQTAAYNLDQVLEQGG | |
| •14-3-3 | VSSLLQNLAVSVTPIQRIVRSGENALKIDIHVIIPYEGLSADQMAQ | |
| •linker | IEEVFKVVYPVDDHHFKVILPYGTLVIDGVTPNMLNYFGRPYEGI | |
| •SmBIT | AVFDGKKITVTGTLWNGNKIIDERLITPDGSMLFRVTINSGGGS | |
| •BAD substrate | MDKNELVQKAKLAEQAERYDDMAACMKSVTEQGAELSNEERN | |
| | LLSVAYKNVVGARRSSWRVVSSIEQKTEGAEKKQQMAREYRE | |
| | KIETELRDICNDVLSLLEKFLIPNASQAESKVFYLKMKGDYYRYL | |
| | AEVAAGDDKKGIVDQSQQAYQEAFEISKKEMQPTHPIRLGLAL | |
| | NFSVFYYEILNSPEKACSLAKTAFDEAIAELDTLSEESYKDSTLI | |
| | MQLLRDNLTLWTSDTGGSGGGGSGGGGSGGGGGGGGSGG | |
| | GGSGGVTGYRLFEPFRGRSRSAPPNLWA | |
| tdTomato | ATGGTGAGTAAGGGCGAGGAAGTGATCAAAGAGTTCATGCG | 11 |
| tKIMBI_Akt | GTTTAAGGTGAGAATGGAAGGAAGCATGAACGGCCACGAGT | |
| Nucleotide | TCGAAATTGAGGGAGAAGGAGAGGGACGGCCCTACGAGGG | |
| Sequence | CACCCAGACAGCCAAGCTGAAAGTGACAAAGGGCGGGCCT | |
| •tdTomato | CTGCCATTCGCTTGGGACATCCTGAGCCCACAGTTTATGTAC | |
| •linker | GGCTCCAAGGCCTATGTGAAACATCCAGCTGACATTCCCGA | |
| •LgBiT | TTATAAGAAACTGAGCTTCCCCGAGGGGTTTAAGTGGGAAA | |
| •linker | GAGTGATGAACTTCGAGGACGGAGGCCTGGTGACTGTGAC | |
| •14-3-3 | CCAGGACAGCTCCCTGCAGGATGGGACCCTGATCTACAAGG | |
| •linker | TGAAAATGAGAGGGACAAATTTTCCCCCTGATGGACCTGTG | |
| •SmBIT | ATGCAGAAGAAAACTATGGGATGGGAGGCCTCCACCGAAAG | |
| •BAD substrate | GCTGTATCCACGCGACGGGGTGCTGAAAGGAGAAATCCACC | |
| | AGGCTCTGAAGCTGAAAGATGGGGGACATTACCTGGTGGAG | |
| | TTCAAGACAATCTACATGGCCAAGAAACCTGTGCAGCTGCC | |
| | AGGCTACTATTACGTGGACACAAAACTGGATATCACTTCACA | |
| | CAACGAGGACTACACTATTGTGGAGCAGTATGAACGGAGCG | |
| | AGGGGAGACACCATCTGTTCCTGGGCCATGGGACTGGAAGT | |
| | ACCGGCTCAGGGTCTAGTGGAACCGCCTCAAGCGAGGATAA | |
| | CAATATGGCTGTGATCAAAGAGTTCATGAGGTTTAAGGTGCG | |
| | CATGGAGGGCAGCATGAATGGGCACGAATTTGAGATTGAAG | |
| | GAGAGGGCGAAGGGAGGCCTTACGAGGGCACACAGACTGC | |
| | CAAGCTGAAAGTGACCAAGGGAGGACCACTGCCTTTCGCTT | |
| | GGGATATCCTGTCTCCTCAGTTTATGTACGGAAGTAAGGCCT | |
| | ATGTCAAGCATCCCGCTGACATTCCTGATTACAAGAAACTGT | |
| | CTTTCCCAGAGGGCTTTAAGTGGGAGAGAGTGATGAATTTT | |
| | GAAGATGGAGGCCTGGTGACCGTGACACAGGACTCCTCTCT | |
| | GCAGGATGGCACTCTGATCTACAAAGTCAAAATGCGCGGCA | |
| | CCAATTTTCCACCCGATGGGCCCGTGATGCAGAAGAAAACA | |
| | ATGGGGTGGGAGGCCAGCACTGAACGGCTGTATCCTAGAG | |
| | ACGGAGTGCTGAAGGGCGAAATCCACCAGGCCCTGAAGCT | |
| | GAAAGACGGCGGCCACTACCTGGTGGAGTTCAAAACCATCT | |
| | ACATGGCCAAGAAACCAGTGCAGCTGCCCGGCTATTACTAT | |
| | GTGGACACCAAGCTGGATATCACATCCCACAATGAAGACTA | |
| | CACCATTGTGGAACAGTATGAGAGGTCTGAAGGACGCCACC | |
| | ATCTGTTTCGGCTGGAAGATTTCGTTGGGGACTGGGAACAG | |
| | ACAGCCGCCTACAACCTGGACCAAGTCCTTGAACAGGGAGG | |
| | TGTGTCCAGTTTGCTGCAGAATCTCGCCGTGTCCGTAACTC | |
| | CGATCCAAAGGATTGTCCGGAGCGGTGAAAATGCCCTGAAG | |
| | ATCGACATCCATGTCATCATCCCGTATGAAGGTCTGAGCGC | |
| | CGACCAAATGGCCCAGATCGAAGAGGTGTTTAAGGTGGTGT | |
| | ACCCTGTGGATGATCATCACTTTAAGGTGATCCTGCCCTATG | |
| | GCACACTGGTAATCGACGGGGTTACGCCGAACATGCTGAAC | |
| | TATTTCGGACGGCCGTATGAAGGCATCGCCGTGTTCGACGG | |
| | CAAAAAGATCACTGTAACAGGGACCCTGTGGAACGGCAACA | |
| | AAATTATCGACGAGCGCCTGATCACCCCCGACGGCTCCATG | |
| | CTGTTCCGAGTAACCATCAACAGCGGCGGAGGTAGTATGGA | |
| | TAAAAATGAGCTGGTTCAGAAGGCCAAACTGGCCGAGCAGG | |
| | CTGAGCGATATGATGACATGGCAGCCTGCATGAAGTCTGTA | |
| | ACTGAGCAAGGAGCTGAATTATCCAATGAGGAGAGGAATCT | |
| | TCTCTCAGTTGCTTATAAAAATGTTGTAGGAGCCCGTAGGTC | |
| | ATCTTGGAGGGTCGTCTCAAGTATTGAACAAAAGACGGAAG | |
| | GTGCTGAGAAAAAACAGCAGATGGCTCGAGAATACAGAGAG | |
| | AAAATTGAGACGGAGCTAAGAGATATCTGCAATGATGTACTG | |
| | TCTCTTTTGGAAAAGTTCTTGATCCCCAATGCTTCACAAGCA | |
| | GAGAGCAAAGTCTTCTATTTGAAAATGAAAGGAGATTACTAC | |
| | CGTTACTTGGCTGAGGTTGCCGCTGGTGATGACAAGAAAGG | |
| | GATTGTCGATCAGTCACAACAAGCATACCAAGAAGCTTTTGA | |
| | AATCAGCAAAAAGGAAATGCAACCAACACATCCTATCAGACT | |
| | GGGTCTGGCCCTTAACTTCTCTGTGTTCTATTATGAGATTCT | |
| | GAACTCCCCAGAGAAAGCCTGCTCTCTTGCAAAGACAGCTT | |
| | TTGATGAAGCCATTGCTGAACTTGATACATTAAGTGAAGAGT | |
| | CATACAAAGACAGCACGCTAATAATGCAATTACTGAGAGACA | |
| | ACTTGACATTGTGGACATCGGATACCGGAGGGAGTGGAGGT | |
| | GGAGGCTCTGGAGGAGGGGGTTCAGGTGGTGGAGGCAGC | |
| | GGCGGAGGTGGATCCGGAGGAGGTGGCAGTGGGGGAGTG | |
| | ACCGGCTACCGGCTGTTCGAGCCATTTCGAGGCCGTTCTAG | |
| | AAGTGCTCCGCCTAACCTCTGGGCA | |
| tdTomato | MVSKGEEVIKEFMRFKVRMEGSMNGHEFEIEGEGEGRPYEGT | 12 |
| tKiMBI_Akt Amino | QTAKLKVTKGGPLPFAWDILSPQFMYGSKAYVKHPADIPDYKK | |
| Acid Sequence | LSFPEGFKWERVMNFEDGGLVTVTQDSSLQDGTLIYKVKMRG | |
| •tdTomato | TNFPPDGPVMQKKTMGWEASTERLYPRDGVLKGEIHQALKLK | |
| •linker | DGGHYLVEFKTIYMAKKPVQLPGYYYVDTKLDITSHNEDYTIVE | |
| •LgBiT | QYERSEGRHHLFLGHGTGSTGSGSSGTASSEDNNMAVIKEFM | |
| •linker | RFKVRMEGSMNGHEFEIEGEGEGRPYEGTQTAKLKVTKGGPL | |
| •14-3-3 | PFAWDILSPQFMYGSKAYVKHPADIPDYKKLSFPEGFKWERVM | |
| •linker | NFEDGGLVTVTQDSSLQDGTLIYKVKMRGTNFPPDGPVMQKK | |
| •SmBIT | TMGWEASTERLYPRDGVLKGEIHQALKLKDGGHYLVEFKTIYM | |
| •BAD substrate | AKKPVQLPGYYYVDTKLDITSHNEDYTIVEQYERSEGRHHLFRL | |
| | EDFVGDWEQTAAYNLDQVLEQGGVSSLLQNLAVSVTPIQRIVR | |
| | SGENALKIDIHVIIPYEGLSADQMAQIEEVFKVVYPVDDHHFKVIL | |
| | PYGTLVIDGVTPNMLNYFGRPYEGIAVFDGKKITVTGTLWNGNK | |
| | IIDERLITPDGSMLFRVTINSGGGSMDKNELVQKAKLAEQAERY | |
| | DDMAACMKSVTEQGAELSNEERNLLSVAYKNVVGARRSSWR | |
| | VVSSIEQKTEGAEKKQQMAREYREKIETELRDICNDVLSLLEKF | |
| | LIPNASQAESKVFYLKMKGDYYRYLAEVAAGDDKKGIVDQSQQ | |
| | AYQEAFEISKKEMQPTHPIRLGLALNFSVFYYEILNSPEKACSLA | |
| | KTAFDEAIAELDTLSEESYKDSTLIMQLLRDNLTLWTSDTGGSG | |
| | GGGSGGGGSGGGGSGGGGSGGGGSGGVTGYRLFEPFRGR | |
| | SRSAPPNLWA | |
| CyOFP1 amino | MVSKGEELIKENMRSKLYLEGSVNGHQFKCTHEGEGKPYEGK | 13 |
| acid sequence | QTNRIKVVEGGPLPFAFDILATHFMYGSKVFIKYPADLPDYFKQ | |
| | SFPEGFTWERVMVFEDGGVLTATQDTSLQDGELIYNVKVRGV | |
| | NFPANGPVMQKKTLGWEPSTETMYPADGGLEGRCDKALKLVG | |
| | GGHLHVNFKTTYKSKKPVKMPGVHYVDRRLERIKEADNETYVE | |
| | QYEHAVARYSNLGGG | |
| tdTomato amino | MVSKGEEVIKEFMRFKVRMEGSMNGHEFEIEGEGEGRPYEGT | 14 |
| acid sequence | QTAKLKVTKGGPLPFAWDILSPQFMYGSKAYVKHPADIPDYKK | |
| | LSFPEGFKWERVMNFEDGGLVTVTQDSSLQDGTLIYKVKMRG | |
| | TNFPPDGPVMQKKTMGWEASTERLYPRDGVLKGEIHQALKLK | |
| | DGGHYLVEFKTIYMAKKPVQLPGYYYVDTKLDITSHNEDYTIVE | |
| | QYERSEGRHHLFLGHGTGSTGSGSSGTASSEDNNMAVIKEFM | |
| | RFKVRMEGSMNGHEFEIEGEGEGRPYEGTQTAKLKVTKGGPL | |
| | PFAWDILSPQFMYGSKAYVKHPADIPDYKKLSFPEGFKWERVM | |
| | NFEDGGLVTVTQDSSLQDGTLIYKVKMRGTNFPPDGPVMQKK | |
| | TMGWEASTERLYPRDGVLKGEIHQALKLKDGGHYLVEFKTIYM | |
| | AKKPVQLPGYYYVDTKLDITSHNEDYTIVEQYERSEGRHHLFRL | |
| mScarlet amino | MVSKGEAVIKEFMRFKVHMEGSMNGHEFEIEGEGEGRPYEGT | 15 |
| acid sequence | QTAKLKVTKGGPLPFSWDILSPQFMYGSRAFIKHPADIPDYYKQ | |
| | SFPEGFKWERVMNFEDGGAVTVTQDTSLEDGTLIYKVKLRGTN | |
| | FPPDGPVMQKKTMGWEASTERLYPEDGVLKGDIKMALRLKDG | |
| | GRYLADFKTTYKAKKPVQMPGAYNVDRKLDITSHNEDYTVVEQ | |
| | YERSEGRHSTG | |
| mScarlet3 amino | MDSTEAVIKEFMRFKVHMEGSMNGHEFEIEGEGEGRPYEGTQ | 16 |
| acid sequence | TAKLRVTKGGPLPFSWDILSPQFMYGSRAFTKHPADIPDYWKQ | |
| | SFPEGFKWERVMNFEDGGAVSVAQDTSLEDGTLIY | |
| | KVKLRGTNFP | |
| | PDGPVMQKKTMGWEASTERLYPEDVVLKGDIKMALRLKDGGR | |
| | YLADFKTTYRAKKPVQMPGAFNIDRKLDITSHNEDYTVVEQYER | |
| | SVARHSTGGSGGS | |
| mCyRFP4 amino | MVSKGEELIKENMRSKLYLEGSVNGHQFKCTAEGEGKPYEGK | 17 |
| acid sequence | QTARIKVVEGGPLPFAFDILAQSFMYGSKVFIKYPADLPDYFKQ | |
| | SFPEGFTWERVMVFEDGGVLTATQDTSLQDGELIYNVKLIGVN | |
| | FPANGPVMQKKTLGWEPSTETMTPADGGLEGRCDKVLKLVGG | |
| | GYLHVNFKTTYKSKKPVKMPGVHYVDRRLERIKEADNETYVEQ | |
| | YEHAVARYSNLGGG | |
| mNeonGreen | NMASLPATHELHIFGSINGVDFDMVGQGTGNPNDGYEELNLKS | 18 |
| amino acid | TKGDLQFSPWILVPHIGYGFHQYLPYPDGMSPFQAAMVDGSG | |
| sequence | YQVHRTMQFEDGASLTVNYRYTYEGSHIKGEAQVKGTGFPAD | |
| | GPVMTNSLTAADWCRSKKTYPNDKTIISTFKWSYTTGNGKRYR | |
| | STARTTYTFAKPMAANYLKNQPMYVFRKTELKHSKTELNFKEW | |
| | QKAFT | |
| mGreenLantern | MVSKGEELFTGVVPILVELDGDVNGHKFSVRGEGEGDATNGKL | 19 |
| amino acid | TLKFICTTGKLPVPWPTLVTTLGYGVACFARYPDHMKQHDFFK | |
| sequence | SAMPEGYVQERTISFKDDGTYKTRAEVKFEGDTLVNRIVLKGID | |
| | FKEDGNILGHKLEYNFNSHKVYITADKQKNGIKANFKTRHNVED | |
| | GGVQLADHYQQNTPIGDGPVLLPDNHYLSHQSKLSKDPNEKR | |
| | DHMVLKERVTAAGITHDMDELYK | |
| LgBiT ΔN2 | FTLEDFVGDWEQTAAYNLDQVLEQGGVSSLLQNLAVSVTPIQR | 20 |
| | IVRSGENALKIDIHVIIPYEGLSADQMAQIEEVFKVVYPVDDHHF | |
| | KVILPYGTLVIDGVTPNMLNYFGRPYEGIAVFDGKKITVTGTLWN | |
| | GNKIIDERLITPDGSMLFRVTINS | |
| SmBIT(ΔC3, | VTGYREFE | 21 |
| L163E) | | |
| SmBIT(ΔC3) | VTGYRLFE | 22 |
| WW phospho- | MADEEKLPPGWEKRMSRSSGRVYYFNHITNASQWERPSGNS | 23 |
| binding moiety | SSGGKNGQGEPAR | |
| amino acid | | |
| sequence | | |
| 14-3-3 phospho- | MDKNELVQKAKLAEQAERYDDMAACMKSVTEQGAELSNEERN | 24 |
| binding moiety | LLSVAYKNVVGARRSSWRVVSSIEQKTEGAEKKQQMAREYRE | |
| amino acid | KIETELRDICNDVLSLLEKFLIPNASQAESKVFYLKMKGDYYRYL | |
| sequence | AEVAAGDDKKGIVDQSQQAYQEAFEISKKEMQPTHPIRLGLAL | |
| | NFSVFYYEILNSPEKACSLAKTAFDEAIAELDTLSEESYKDSTLI | |
| | MQLLRDNLTLWTSDT | |
| FHA1 phospho- | NSMHKFSQEQIGENIVCRVICTTGQIPIRDLSADISQVLKEKRSIK | 25 |
| binding moiety | KVWTFGRNPACDYHLGNISRLSNKHFQILLGEDGNLLLNDISTN | |
| amino acid | GTWLNGQKVEKNSNQLLSQGDEITVGVGVESDILSLVIFINDKF | |
| sequence | KQCLEQNKVDR | |
| FHA2 phospho- | AGTKGNGRFLTLKPLPDSIIQESLEIQQGVNPFFIGRSEDCNCKI | 26 |
| binding moiety | EDSRLSRVHCFIFKKRHAVGKSMYESPAQGLDDIWYCHTGTNV | |
| amino acid | SYLNNNRMIQGTKFLLQDGDEIKIIWDKNNKFVIGFKVEINDTTG | |
| sequence | LFNEGLGMLQEQRVVLKQTAEEKDLVKKL | |
| ERK substrate | PDSPRTPVDKAKLSFQFP | 27 |
| MEK1 substrate | ISGYLVDSVAKTIDA | 28 |
| MEK2 substrate | HDHTGFLTEYVATRW | 29 |
| CDK1 substrate | RNSPVTKTPPRDLPT | 30 |
| CDK4/CDK6 | AVIPINGSPRTPRRG | 31 |
| substrate | | |
| JNK substrate | ETRSRHSSYPAGTEE | 32 |
| p38 substrate | ANLNRSTSVPENPKS | 33 |
| mTOR substrate | FTVGTPPSG | 34 |
| EGFR substrate | NFANFSAYPSEEDMI | 35 |
| HER2 substrate | AAEEIYAARRG | 36 |
| VEGFR1 substrate | GIAVPDVYSACKRFE | 37 |
| FGFR1 substrate | CQSCVSEYKQVGSHP | 38 |
| PDFGRA substrate | RLIEDNEYTARQGAK | 39 |
| Aurora A substrate | PAQPQRRSLRLSAQKDLEQ | 40 |
| KIT substrate | SETKKGFYSLSVRHR | 41 |
| AKT substrate | PFRGRSRTAPANLWA | 42 |
| AKT substrate | RLGERLRTMSCSDKI | 43 |
| AKT substrate | GQRDRSSSAPNVHIN | 44 |
| PIK3CA substrate | KDGATMKTFCGTPEY | 45 |
| BRAF substrate | EIRSRHSSYPAGTED | 46 |
| ABL1 substrate | PPTVPNDYMTSPARL | 47 |
| BCR-ABL1 | IPTSAPVYQQPQQQP | 48 |
| substrate | | |
| RET substrate | TFCGTPEYLAPEVLE | 49 |
| EPHA2 substrate | GIAVPDVYSACKRFE | 37 |
| EPHA5 substrate | GIAVPDVYSACKRFE | 37 |
| CHK2 substrate | SKKSRSHSAKTPGFS | 50 |
| PDK1 substrate | KDGATMKTFCGTPEY | 45 |
| ALK substrate | DPGSAAPYLKTKFIC | 51 |
| BTK substrate | EILSRRPSYRKILND | 52 |
| MET substrate | TEADGELYTENTPSG | 53 |
| JAK1 substrate | DGPKGTGYIKTELIS | 54 |
| Src substrate | VTQVSSAYQKTVPVE | 55 |
| ROCK substrate | IIGLQMGTNKFASQQ | 56 |
| LRRK2 substrate | RLGWWRFYTLRRARQGNTKQR | 57 |
| LRRK2 substrate | MAKTYDYLFKLLLIGDSGVGKTCVLFRFSEDAFNSTFISTIGIDFK | 58 |
| (full-length Rab8a) | IRTIELDGKRIKLQIWDTAGQERFRTITTAYYRGAMGIMLVYDITN | |
| | EKSFDNIRNWIRNIEEHASADVEKMILGNKCDVNDKRQVSKER | |
| | GEKLALDYGIKFMETSAKANINVENAFFTLARDIKAKMDKKLEG | |
| | NSPQGSNQGVKITPDQQKRSSFFRCVLL | |
| LRRK2 substrate | MAKKTYDLLFKLLLIGDSGVGKTCVLFRFSDDAFNTTFISTIGIDF | 59 |
| (full-length Rab10) | KIKTVELQGKKIKLQIWDTAGQERFHTITTSYYRGAMGIMLVYDI | |
| | TNGKSFENISKWLRNIDEHANEDVERMLLGNKCDMDDKRVVP | |
| | KGKGEQIAREHGIRFFETSAKANINIEKAFLTLAEDILRKTPVKEP | |
| | NSENVDISSGGGVTGWKSKCC | |
| LRRK2 substrate | MNPEYDYLFKLLLIGDSGVGKSCLLLRFADDTYTESYISTIGVDF | 60 |
| (full-length Rab1b) | KIRTIELDGKTIKLQIWDTAGQERFRTITSSYYRGAHGIIVVYDVT | |
| | DQESYANVKQWLQEIDRYASENVNKLLVGNKSDLTTKKVVDNT | |
| | TAKEFADSLGIPFLETSAKNATNVEQAFMTMAAEIKKRMGPGA | |
| | ASGGERPNLKIDSTPVKPAGGGCC | |

The present disclosure provides each of the KiMBI polypeptides and KiMBI domains provided in Table 1, as well as the nucleic acids that encode such polypeptides and individual domains. Cells comprising such polypeptides and nucleic acids are also provided. As will be appreciated, the present disclosure also provides variants of any of the polypeptides and individual domains therein, where in some instances a variant polypeptide or domain thereof comprises an amino acid sequence having 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, or 99% or greater amino acid sequence identity to the parental/reference sequence, or a functional fragment thereof, where the variant retains the functionality (e.g., capability for enzyme fragment complementation, bioluminescence, phospho-binding, phosphorylatability, and/or the like) of the parental/reference sequence.

For example, in certain embodiments, variants of such polypeptides having one or more (e.g., 1, 2, 3, 4, 5, 6, 7, 8, 9, or 10) amino acid substitutions are provided. Conservative substitutions are shown in the table below under the heading of “preferred substitutions.” More substantial changes are provided in the table below under the heading of “exemplary substitutions,” and as further described below in reference to amino acid side chain classes. Amino acid substitutions may be introduced into a polypeptide of interest and the products screened for a desired activity, e.g., capability for enzyme fragment complementation, bioluminescence, phospho-binding, phosphorylatability, and/or the like.

### Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gin (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

Because of the knowledge of the codons corresponding to the various amino acids, availability of an amino acid sequence of a polypeptide of interest provides a description of all the polynucleotides capable of encoding the polypeptide. The degeneracy of the genetic code, where the same amino acids are encoded by alternative or synonymous codons allows an extremely large number of nucleic acids to be made, all of which encode the polypeptides disclosed herein. Thus, having identified a particular amino acid sequence, those of ordinary skill in the art could make any number of different nucleic acids by simply modifying the sequence of one or more codons in a way which does not change the amino acid sequence of the polypeptide of interest. In this regard, the present disclosure specifically contemplates each and every possible variation of polynucleotides that could be made by selecting combinations based upon the possible codon choices, and all such variations are to be considered specifically disclosed for any polypeptide disclosed herein. As such, also encompassed by the present disclosure are nucleic acids encoding any of the polypeptides or domains thereof of the present disclosure, which differ from a nucleotide sequence in Table 1 above by virtue of the degeneracy of the genetic code.

The nucleotide sequences of the nucleic acids of the present may be codon-optimized. “Codon-optimized” refers to changes in the codons of the polynucleotide encoding a polypeptide to those preferentially used in a cell of a particular organism such that the encoded protein is efficiently expressed in the cell. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, a nucleic acid of the present disclosure encoding a polypeptide may be codon-optimized for optimal expression in a cell of a particular organism, e.g., rodent cells, non-human primate cells, or the like.

Also provided are expression constructs comprising any of the nucleic acids of the present disclosure. As used herein, an “expression construct” is a circular or linear polynucleotide (a polymer composed of naturally occurring and/or non-naturally occurring nucleotides) comprising a region that encodes a polypeptide of the present disclosure, operably linked to a suitable promoter, e.g., a constitutive or inducible promoter. In some embodiments, expression of the polypeptide is under the control of one or more exogenous (including heterologous) regulatory elements, e.g., promoter, enhancer, etc., present in the expression construct. In some embodiments, expression of the polypeptide may be controlled by one or more endogenous regulatory elements, e.g., promoter, enhancer, etc., at or near a genomic locus into which the expression construct is inserted.

The expression constructs (e.g., vectors) can be suitable for replication and integration in prokaryotes, eukaryotes, or both. The expression constructs may contain functionally appropriately oriented transcription and translation terminators, initiation sequences, and promoters useful for regulation of the expression of the nucleic acid encoding the first subunit, the second subunit, or both. The expression constructs optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression constructs which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing the polypeptide are available using, for example, E. coli, Bacillus sp. and Salmonella. E. coli systems may also be used. Transducing cells with nucleic acids (e.g., expression constructs) can involve, for example, incubating lipidic microparticles containing nucleic acids with cells or incubating viral vectors containing nucleic acids with cells within the host range of the vector.

In certain embodiments, upon delivery of an expression construct to cells, one or more of the expression constructs are episomal (e.g., extra-chromosomal), where by “episome” or “episomal” is meant a polynucleotide that replicates independently of the cell's chromosomal DNA. A non-limiting example of an episome that may be employed is a plasmid.

According to some embodiments, upon delivery of an expression construct to cells, the expression construct integrates into the genome of the cell. In certain embodiments, the expression construct is adapted for site-specific integration into the genome. Functional integration of an expression construct may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the cells in a suitable transduction medium, at a suitable concentration (or multiplicity of infection), and for a suitable time for the vector to infect the target cells, facilitating functional integration of the expression construct. Non-limiting examples of useful viral vectors include retroviral vectors, lentiviral vectors, adenoviral (Ad) vectors, adeno-associated virus (AAV) vectors, hybrid Ad-AAV vector systems, and the like.

Also provided by the present disclosure are messenger RNAs (mRNAs) encoding any of the polypeptides of the present disclosure. In some instances, the mRNAs are encapsulated within particles, e.g., lipid nanoparticles. Such mRNAs and particles find use, e.g., for expression of a KiMBI polypeptide in vivo upon delivery of the mRNA to cells of a non-human animal (e.g., rodent or the like). Strategies for delivering mRNAs to cells in vivo are described, e.g., in Kowalski et al. (2019) Mol Ther. 27(4):710-728; and Hou et al. (2021) Nature Reviews Materials 6:1078-1094.

Aspects of the present disclosure further include cells comprising a nucleic acid of the present disclosure, as well as cells comprising an expression construct of the present disclosure. In certain embodiments, the cells are prokaryotic cells (e.g., bacteria), a yeast cells, insect (e.g., drosophila) cells, amphibian (e.g., frog, e.g., Xenopus) cells, plant cells, etc. According to some embodiments, the cells are mammalian cells. Mammalian cells of interest include human cells, rodent cells, and the like.

Additional aspects of the present disclosure include non-human animals comprising any of the cells of the present disclosure. In some embodiments, the cells are endogenous to the non-human animal, e.g., in instances in which the expression construct was introduced into the endogenous cells of the non-human animal. In other embodiments, the cells are exogenous to the non-human animal, e.g., in instances in which the cells were introduced into the non-human animal. In certain embodiments, a non-human animal of the present disclosure is a mammal. Mammals of interest include, but are not limited to, a rodent (e.g., a mouse or a rat), a non-human animal primate, and the like.

### Methods of Use

Aspects of the present disclosure further include methods of using the nucleic acids and KiMBI polypeptides of the present disclosure. In certain embodiments, provided are methods of assessing activity of a kinase of interest in a living non-human animal, wherein the non-human animal comprises cells that comprise any of the nucleic acids of the present disclosure (optionally comprised within an expression construct, integrated into the genome of the cell and operably linked to an endogenous promoter within the genome, and/or the like), the cells co-express the KiMBI polypeptide and the kinase of interest, and the kinase substrate of the KiMBI polypeptide is a substrate for the kinase of interest. Such methods comprise administering to the non-human animal a substrate for the bioluminescent enzyme, wherein the substrate enters the cells co-expressing the KiMBI polypeptide and the kinase of interest. Such methods further comprise assessing the cells for bioluminescence catalyzed by the bioluminescent enzyme, wherein bioluminescence is inversely related to activity of the kinase of interest.

According to some embodiments, provided are methods of assessing a test agent for the ability to inhibit a kinase of interest in a living non-human animal, wherein the non-human animal comprises cells that comprise any of the nucleic acids of the present disclosure (optionally comprised within an expression construct, integrated into the genome of the cell and operably linked to an endogenous promoter within the genome, and/or the like), the cells co-express the KiMBI polypeptide and the kinase of interest, and the kinase substrate of the KiMBI polypeptide is a substrate for the kinase of interest. Such methods comprise administering to the non-human animal the test agent and a substrate for the bioluminescent enzyme, wherein the substrate enters the cells co-expressing the KiMBI polypeptide and the kinase of interest. Such methods further comprise assessing the cells for bioluminescence catalyzed by the bioluminescent enzyme, wherein bioluminescence is positively related to the ability of the test agent to inhibit the kinase of interest. In some instances, the test agent is a small molecule. By “small molecule” is meant a compound having a molecular weight of 1000 atomic mass units (amu) or less. In some embodiments, the small molecule is 900 amu or less, 750 amu or less, 500 amu or less, 400 amu or less, 300 amu or less, or 200 amu or less. In some instances, the small molecule is not made of repeating molecular units such as are present in a polymer.

According to some embodiments of the methods, the kinase of interest is a tyrosine kinase, external signal-regulated kinase (ERK), mitogen-activated protein kinase kinase (MEK), a cyclin-dependent kinase (CDK), an aurora kinase, a mitogen-activated protein kinase, ERBB1 (EGFR), ERBB2 (HER2), VEGFR, FGFR, Kit, PDGFR, AKT1/AKT2/AKT3, PIK3CA, BRAF, mTOR, SRC, ABL1, BCR-ABL, RET, AATK, EPHA2, EPHA5, CHK2, PKD1, ALK, BTK, MET, KIT, JAK, MEK1/2, Src, ROS1, ROCK, CDK4/CDK6, PI3Kδ, or LRRK2.

The substrate administered to the non-human animal is selected based on the bioluminescent enzyme employed in the KiMBI polypeptide. In some instances, the substrate for the bioluminescent enzyme is fluorofurimazine (FFz) or cephalofurimazine (CFz), e.g., when the bioluminescent enzyme is a luciferase, such as a NanoLuc (NLuc) luciferase. The substrate for the bioluminescent enzyme may be administered to the non-human animal via any convenient route of administration. In some instances, the substrate is administered via parenteral administration, e.g., via intravenous administration, via intraperitoneal (i.p.) administration, via local administration to a site of the non-human animal of interest, etc. Detailed guidance for formulating and administering a substrate for a bioluminescent enzyme is provided in the Experimental section below.

In some embodiments, the cells that co-express the KiMBI polypeptide and the kinase of interest are endogenous to the non-human animal, e.g., in instances in which the nucleic acid encoding the KiMBI was delivered to endogenous cells of the non-human animal. Delivery of the nucleic acid encoding the KiMBI to endogenous cells of the non-human animal may be achieved through various means, including through the use of non-integrating vectors or integrating vectors, including viral and non-viral vectors. In some instances, a retroviral vector, e.g., a lentiviral vector, may be employed. In some instances, a non-retroviral integrating vector may be employed. An integrating vector may be administered to the non-human animal to infect the target cells, facilitating functional integration of the expression construct. Non-limiting examples of useful viral vectors include retroviral vectors, lentiviral vectors, adenoviral (Ad) vectors, adeno-associated virus (AAV) vectors, hybrid Ad-AAV vector systems, and the like. In some instances, the nucleic acids encoding the KiMBI are messenger RNAs (mRNAs). In certain embodiments, the mRNAs are encapsulated within particles, e.g., lipid nanoparticles, and the particles are administered to the non-human animal to deliver the mRNAs to target cells for expression of the KiMBI therein. Strategies for delivering mRNAs to cells in vivo are described, e.g., in Kowalski et al. (2019) Mol Ther. 27(4):710-728; and Hou et al. (2021) Nature Reviews Materials 6:1078-1094.

In certain embodiments, the cells that co-express the KiMBI polypeptide and the kinase of interest are exogenous to the non-human animal. For example, in some instances, cells that co-express the KiMBI polypeptide and the kinase of interest are produced in vitro and then administered to the non-human animal, e.g., by infusion or administered/implanted locally to a site of the non-human animal of interest. Example approaches for implanting cells at a site of interest in a non-human animal are described in the Experimental section below. In certain embodiments, the cells are human cells. In some instances, the cells are cancer cells, e.g., human cancer cells.

According to some embodiments, the cells that co-express the KiMBI polypeptide and the kinase of interest are tumor cells. By “tumor cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Tumor cell” may be used interchangeably herein with “cancer cell”, “malignant cell” or “cancerous cell”, and encompasses cells of a solid tumor, a semi-solid tumor, a primary tumor, a metastatic tumor, and the like. In certain embodiments, the tumor cell is a carcinoma cell, a lymphoma, a blastoma cell, or a sarcoma cell.

In some instances, the cells that co-express the KiMBI polypeptide and the kinase of interest are cells of a cancer selected from renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgendependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas.

According to some embodiments, the cells that co-express the KiMBI polypeptide and the kinase of interest are present in the brain of the non-human animal. In certain embodiments, the cells that co-express the KiMBI polypeptide and the kinase of interest are tumor cells. In one non-limiting example, the tumor cells are glioblastoma cells.

In certain embodiments of the methods, the non-human animal is a mammal. Mammals of interest include, but are not limited to, a rodent (e.g., a mouse or a rat), and a non-human animal primate.

The methods of the present disclosure include assessing the cells for bioluminescence catalyzed by the bioluminescent enzyme. Exemplary procedures for in vivo bioluminescence imaging are described in detail in the Experimental section below. For example, images may be acquired (e.g., using an Ami HT optical imaging system) at a suitable frequency (e.g., every 1 min) for a suitable duration (e.g., for 10 min), with an air flow containing 1-2% isoflurane for stable anesthesia. Suitable imager settings include, e.g.: emission filter, open; field of view, 25 cm; f-stop 1.2; binning, 2×2 and exposure time, 30 s. Images may be analyzed using available in vivo imaging software, e.g., Aura 4.0 in vivo imaging software by way of example.

The following examples are offered by way of illustration and not by way of limitation.

### EXPERIMENTAL

### Example 1—Development and Optimization of ERK KiMBIs

Reported in the Examples herein is a genetically encoded Kinase-Modulated Bioluminescent Indicator (sometimes referred to herein as “KiMBI”) for real-time non-invasive visualization of kinase inhibition in living animals. As pathways activating external signal-regulated kinase (ERK) are frequently up-regulated in human tumors, a KiMBI for ERK inhibition was designed as a first example. Through extensive engineering of the luciferase NanoLuc, a KiMBI with a 10-fold response to ERK pathway inhibition was obtained. ERK KiMBI expressed in the mouse brain successfully differentiated between brain permeant and impermeant inhibitors of ERK or its upstream activator, mitogen-activated protein kinase kinase (MEK). ERK KiMBI also revealed real-time drug inhibition of the ERK pathway within human tumor xenografts outside or inside the mouse brain. Finally, by longitudinal imaging of KiMBI signals, the kinetics of kinase inhibition could be assessed following each injection, a task that would be highly resource-intensive using traditional animal sampling approaches.

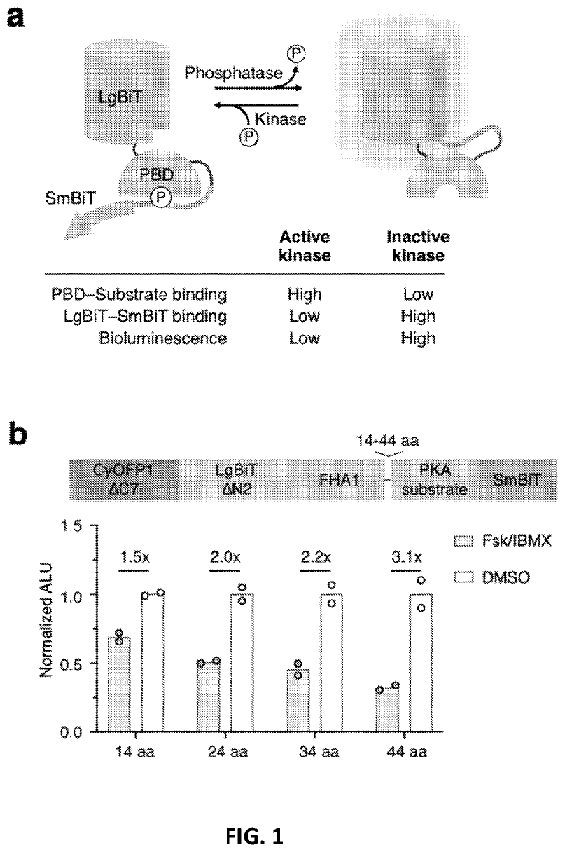

To develop a method to produce light upon kinase inhibition, it was hypothesized that the catalytic activity of NanoLuc luciferase (as proof of principle) could be modulated through interactions between phosphorylated substrate (pSub) and phosphopeptide-binding domains (PBDs). The NanoBit version of NanoLuc can be split after amino acid 156 in the loop before the last beta strand, producing complementing fragments LgBiT and SmBiT, with SmBiT available as different affinity variants. It was postulated that inserting a PBD (or kinase substrate) in between LgBiT and SmBiT, and fusing the cognate substrate (or PBD) elsewhere in the protein, could confer dephosphorylation-dependence on the enzymatic activity of NanoBiT. Specifically, when a phosphorylating kinase is highly active, the PBD-pSub interaction could tether the SmBiT in a conformation incompatible with binding to LgBiT, effectively competing with the weaker LgBiT-SmBiT interaction. In contrast, if the kinase or an upstream kinase is inhibited by a kinase inhibitor, then ongoing dephosphorylation could shift the equilibrium of conformational states toward LgBiT-SmBiT interaction, resulting in light production (FIG. 1a).

The design hypothesis was first tested using a PKA substrate and a cognate FHA domain. Constructs of the topology LgBiT-FHA-linker-substrate-SmBiT were designed, with various linker lengths to allow the phosphorylated substrate to reach its binding site on FHA when phosphorylated. Proteins were expressed in mammalian cells, which were then treated with forskolin and IBMX to activate endogenous PKA. All constructs showed lower bioluminescence with PKA activation, with the longest linker (44 amino acids) producing the largest induction (FIG. 1b). These results demonstrate that bioluminescent indicators of kinase inhibition can indeed be engineered on the basis of PBD-pSub binding competing with LgBiT-SmBiT binding.

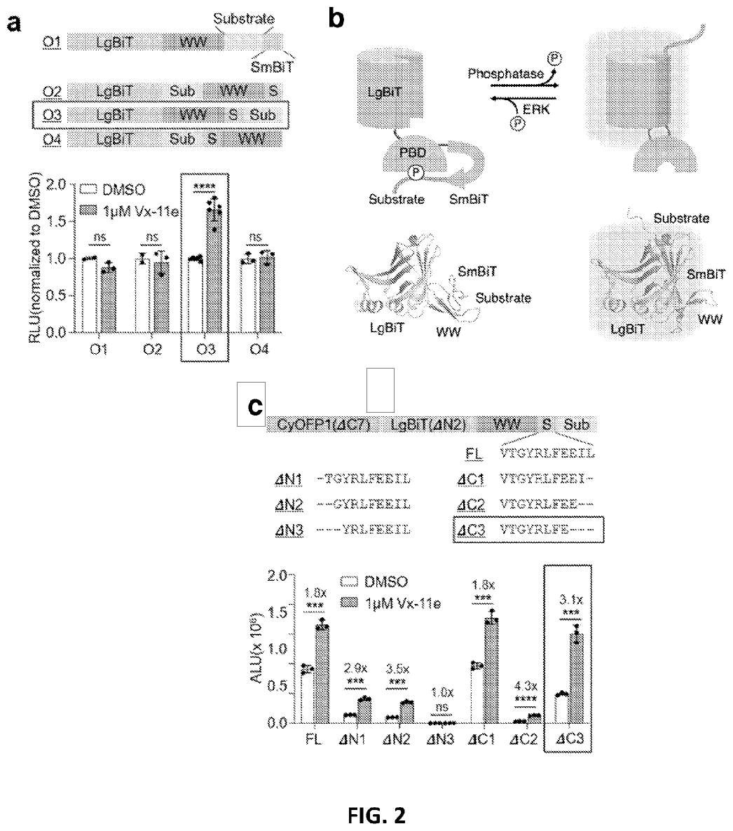

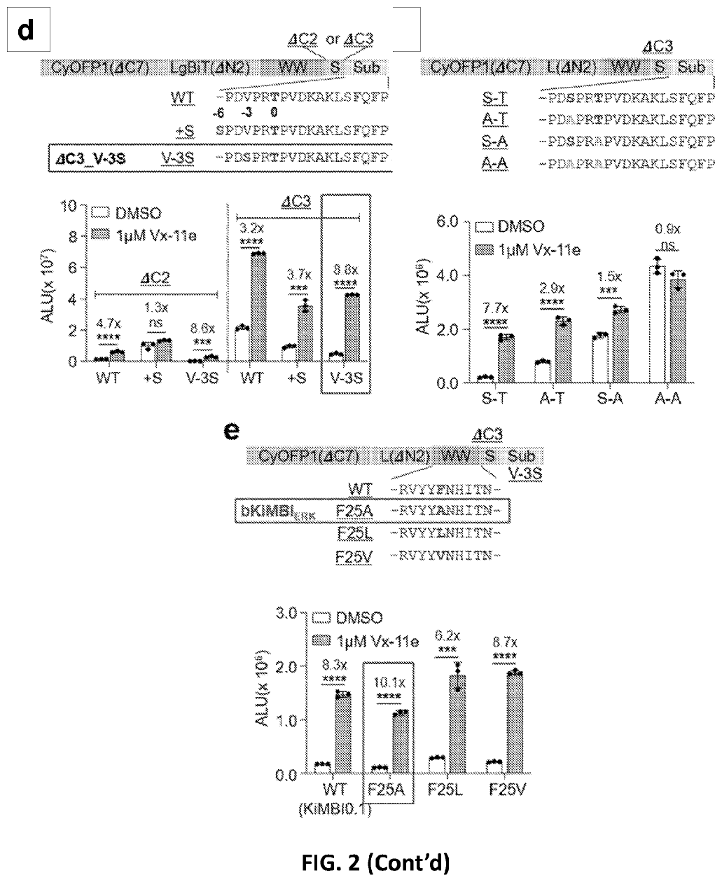

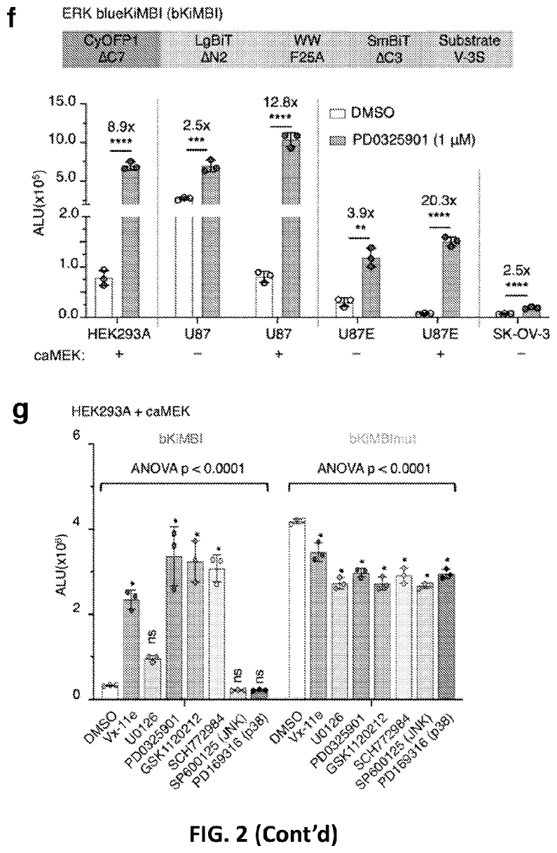

To develop a luciferase reporter protein whose activity can be activated by ERK inhibition, it was hypothesized that NanoLuc luciferase catalytic activity could be modulated through large ERK-induced conformational changes. Created and tested were fusion proteins of various topologies (FIG. 2a) comprising the NanoLuc NanoBiT fragments, an ERK substrate peptide from Cdc25C⁷, and a proline-directed WW phospho-binding domain (WW)⁸ from Pin1. In particular, an arrangement was postulated in which binding of phosphorylated substrate (pSub) to WW would force the SmBiT fragment to adopt an unfavorable conformation for LgBiT complementation, would show low luminescence with active ERK and high luminescence with inhibited ERK (FIG. 2b). The KiMBI candidates were screened by co-transfection into HEK293A cells with a constitutively active MEK (caMEK) followed by application of the ERK inhibitor Vx-Ile. Indeed, the 03 topology showed a significant increase in luminescence (AUL) of ˜65% when treated with 1 μM Vx-11e (FIG. 2a). This suggests that ERK inactivation is followed by dephosphorylation of the substrate and its dissociation from the WW domain, allowing LgBiT-SmBiT complementation and luciferase activity restoration (FIG. 2b, right).

Next, the dynamic range of the 03 KiMBI candidate was improved. Based on the proposed mechanism of action, it was reasoned that disfavoring LgBiT-SmBiT complementation or favoring WW-phosphosubstrate binding could lower basal luminescence when ERK is active, thus improving fold induction when ERK is inhibited. Screening truncated or mutated SmBiT variants, it was found that truncation of 3 amino acids (aa) from the SmBiT C-terminus (CT) improved the response to ˜300% (ΔC3, FIG. 2c). ΔC3 was further improved by introducing a second ERK phosphorylation site at position −3 within the Cdc25C substrate sequence to increase WW-phosphosubstrate avidity. This change improved the induction to 8.8-fold (ΔC3 V-3S, FIG. 2d, left). Substitution of either the additional (serine, position −3) or original (threonine, position 0) phosphorylation sites with alanine significantly decreased the drug response, suggesting that both sites are phosphorylated by ERK and recognized by WW domain (FIG. 2d, right). Finally, WW-phosphosubstrate binding was further enhanced by introducing a F25A mutation in the WW domain⁹ (FIG. 2e). The resulting construct showed >10-fold induction by Vx-Ile, and was named ERK blue KiMBI (bKiMBI, FIG. 2e).

bKiMBI function was validated in cancer cell lines where ERK is hyperactive, selecting U87 (human glioma), U87-EGFRvIII (U87 overexpressing epidermal growth factor receptor variant III, which further activates ERK), and SK-OV-3 (human ovarian cancer). bKiMBI increased brightness after ERK inhibition, with a ˜2.5-fold response. U87-EGFRvIII exhibited lower basal KiMBI signal than U87, consistent with the presence of the constitutive ERK activator EGFRvIII (FIG. 2f). Endogenous ERK activity in these cell lines appeared to be only partially activated, as co-expressing caMEK further suppressed basal KiMBI signal. Next, it was found that ERK bKiMBI was activated by multiple MEK or ERK inhibitors, but not by any JNK or p38 inhibitors (FIG. 2g). This provides additional evidence that KiMBI responds positively to ERK inhibition, and argues against inhibitors directly binding KiMBIs to enhance NanoLuc assembly or catalysis. As a control, the effects of kinase inhibitors on a phosphosite mutant of ERK bKiMBI were also tested; this revealed slightly lower luminescence after adding most kinase inhibitors, likely due to lower protein expression levels. Positive KiMBI responses must actually overcome this tendency of kinase inhibitors to suppress bioluminescence.

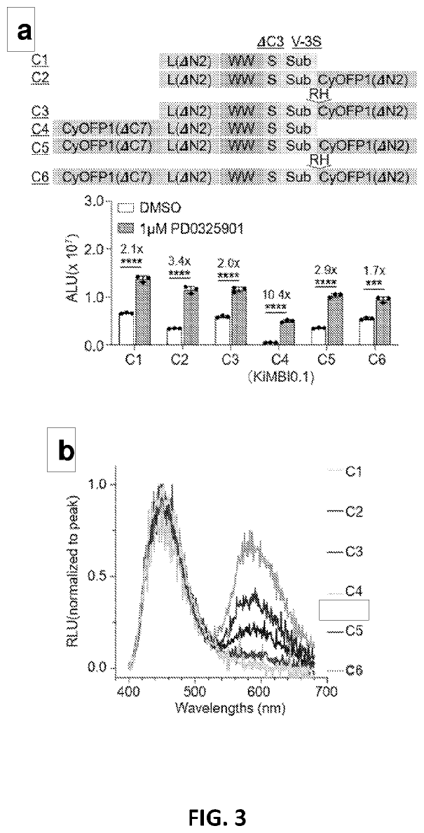

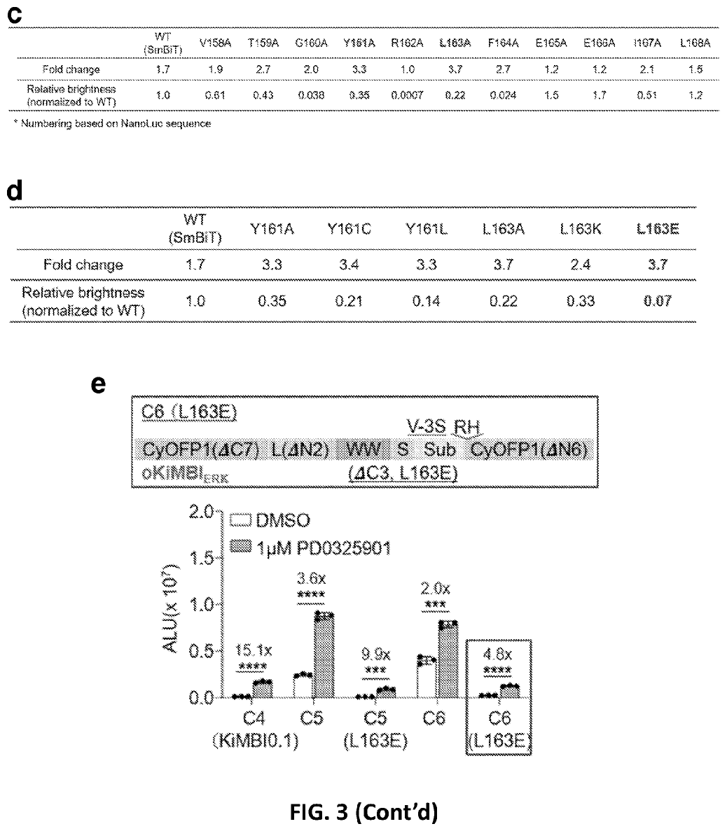

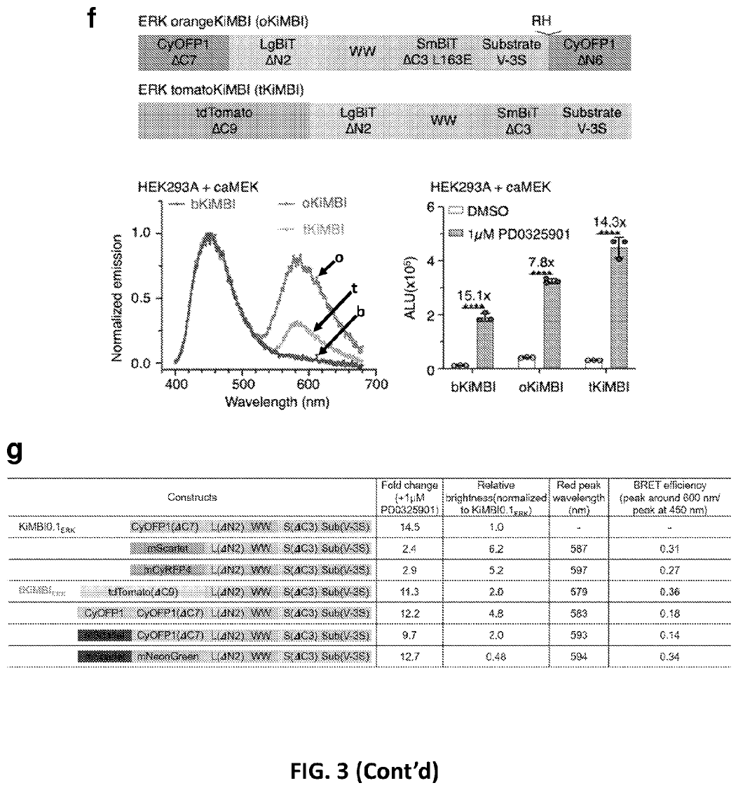

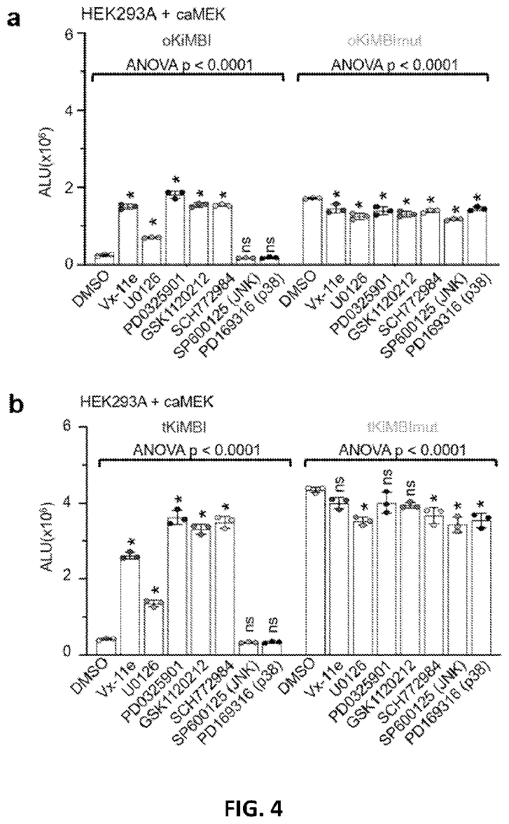

Next, to red-shift KiMBI emission for better tissue penetration in vivo, fusing fluorescent proteins to bKiMBI to allow resonance energy transfer (RET) was explored. Although constructs in the early rounds of engineering included a CyOFP1¹⁰ at their N-terminus, there was no detectable RET between the reconstituted NanoBiT and CyOFP1 (FIG. 3a,b). Following the example of Antares luciferase¹⁰, a second CyOFP1 domain was fused to the C-terminus of the ΔC3 V-3S KiMBI precursor. Only the additional fusion with a 2-aa linker improved RET efficiency, but it also increased luminescence in active ERK conditions and diminished the response to ERK inhibition (C6, FIG. 3a,b). To rescue the response, the ability of SmBiT to bind LgBiT was weakened by performing alanine scanning followed by further mutagenesis, resulting in an optimized L163E mutation (FIG. 3c-e). The resulting construct, named orange KiMBI (oKiMBI), maintained high RET while restoring most of the inducibility of bKiMBI (FIG. 3f). Additional potential RET acceptors fused to the N-terminus of LgBiT were tested (FIG. 3g). Fusion with tdTomato allowed a high amount of RET and even better inducibility by inhibitor (FIG. 3f,g), and was named tomato KiMBI (tKiMBI). oKiMBI and tKiMBI were activated by all MEK or ERK inhibitors tested, but not by JNK or p38 inhibitors (FIG. 4), reconfirming specificity for ERK inhibitors.

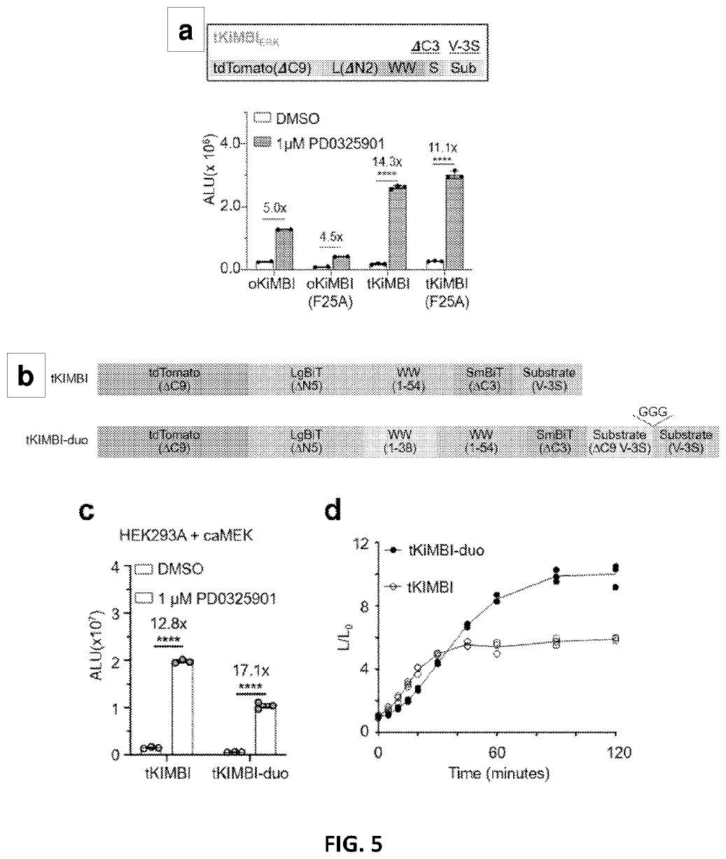

Whether oKiMBI or tKiMBI responsiveness could be further improved was explored. The F25A mutation that had improved bKiMBI responses did not improve oKiMBI or tKiMBI responses, so Phe-25 was retained in these variants (FIG. 5a). Also assessed was whether duplicating WW domain and substrate could further repress tKiMBI bioluminescence and allow larger changes with ERK inhibition (FIG. 5b). The resulting tKiMBI-duo exhibited lower brightness in active ERK conditions and modestly higher fold induction, but at the cost of peak brightness in inactive ERK conditions (FIG. 5c). Also assessed was the relationship between basal suppression and speed of ERK tKiMBI responses. As tighter WW-phosphosubstrate interactions reduce the fraction of time in which the phosphosubstrate is exposed for dephosphorylation by phosphatases, they may reduce the speed of KiMBI induction by kinase inhibitors as well. Indeed, it was observed that the transition to full induction of tKiMBI occurred over ˜1 h, while tKiMBI-duo required 2 h for full induction (FIG. 5d). For better time resolution, the simpler tKiMBI was employed in subsequent experiments.

### Example 2—Molecular Imaging of ERK Inhibition in a Subcutaneous Tumor Model

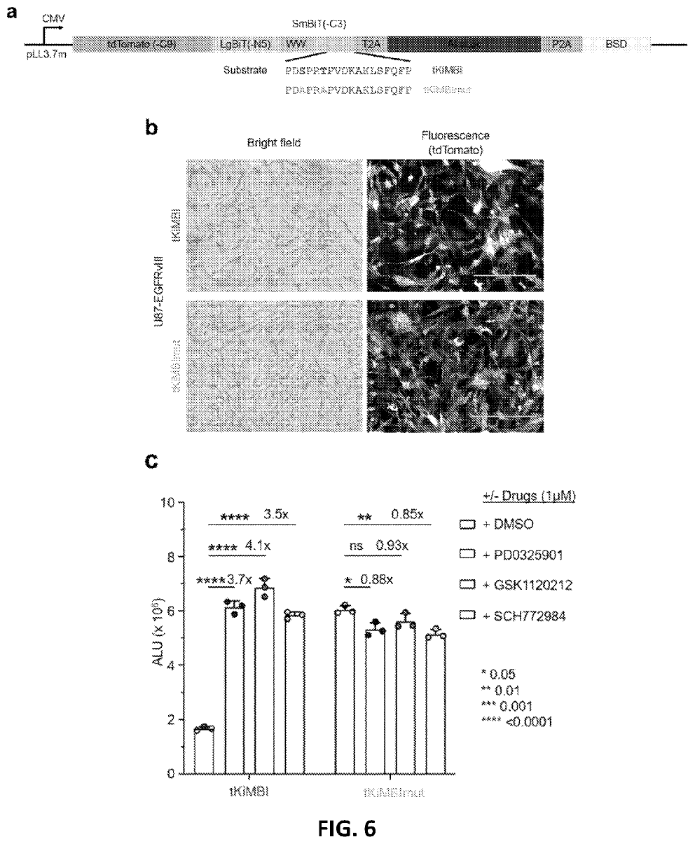

Next, the ability of KiMBIs to report ERK kinase inhibition in human tumor xenografts in mice was tested. U87-EGFRvIII cells were transduced with lentiviruses expressing tKiMBI-T2A-AkaLuc, where AkaLuc allows kinase-independent tracking of tumor location and size (FIG. 6a). Transduced cells were sorted by fluorescence-activated cell sorting (FACS) and selected by antibiotics to generate polyclonal stable cell lines. A control reporter cell line expressing tKiMBIₘᵤₜ was generated in parallel, and expression of the indicators was confirmed by fluorescence microscopy (FIG. 6b). Treatment of tKiMBI-expressing cells with PD0325901 (MEK), GSK1120212 (MEK), or SCH772984 (ERK) induced bioluminescence by ˜4-fold, whereas treatment of the control cell lines slightly reduced bioluminescence (FIG. 6c). Overall, tKiMBI-expressing stable cell lines responded to MEK or ERK inhibitors similarly to transiently transfected cells.

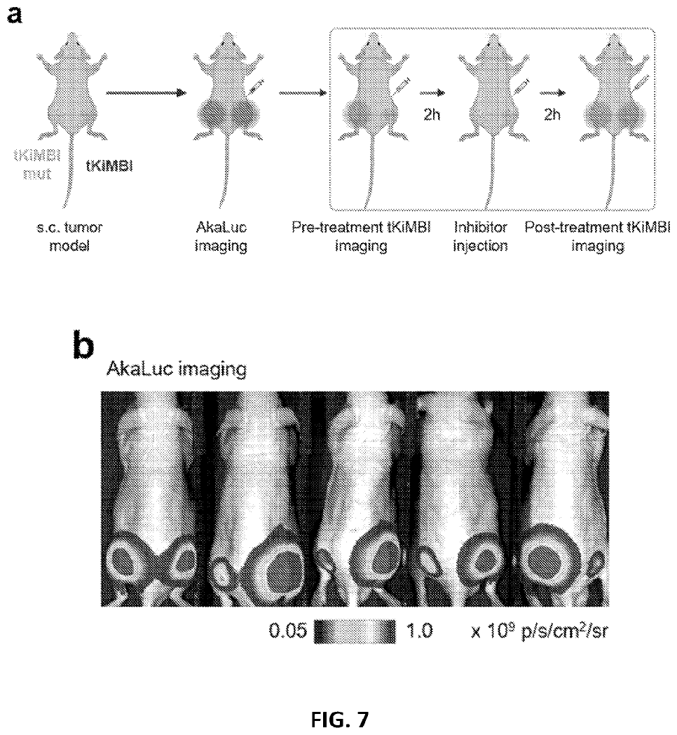

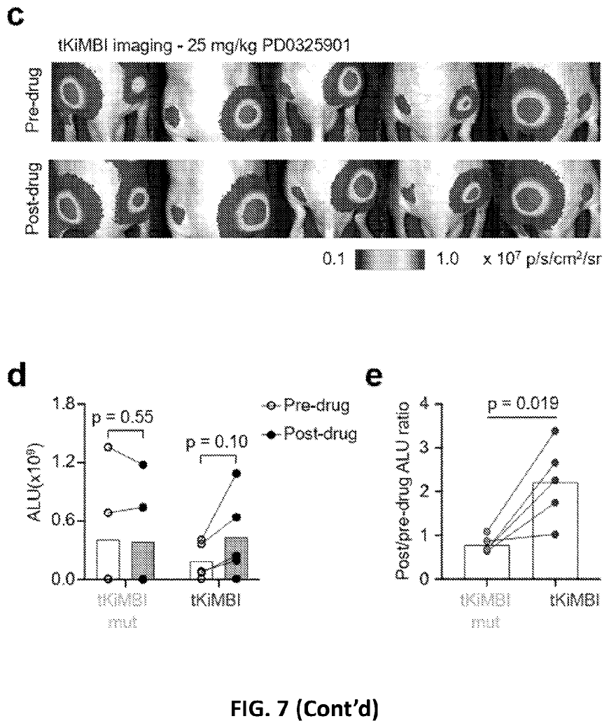

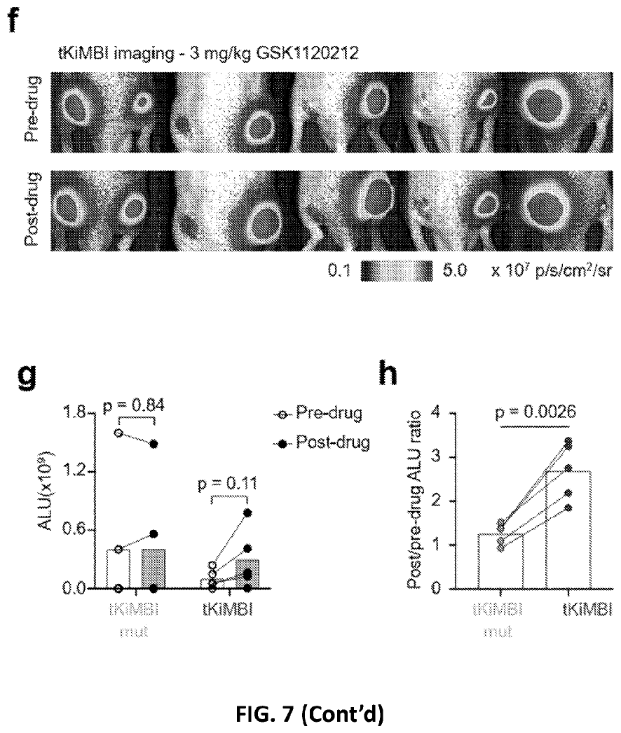

The ability of tKiMBI to report ERK pathway inhibition in tumors in vivo was assessed. Mice were injected subcutaneously with 10⁶ tKiMBI-expressing cells on one side and, to control for differences in substrate delivery between injections, tKiMBIₘᵤt-expressing reporter cells on the other side (FIG. 7a). One week after implantation, sizes of the engrafted tumors were quantified by measuring peak bioluminescence after injection of AkaLumine, the AkaLuc substrate (FIG. 7b). tKiMBI bioluminescence was then imaged before or 2 h after injection of MEK inhibitor PD0325901 by administering the NanoLuc substrate fluorofurimazine (FFz)¹¹. A clear signal increase was seen after PD0325901 in the tKiMBI-expressing tumor, but not in the tKiMBIₘᵤt-expressing tumor (FIG. 7c-d). On average, tKiMBI responded to PD0325901 with a 100% increase in brightness, significantly different from the ˜10% of tKiMBIₘᵤₜ (FIG. 7e). This result illustrates the ability of tKiMBI to report ERK inhibition within tumor cells in vivo.

One of the advantages of non-invasive tKiMBI imaging is that multiple kinase inhibitor candidates can be tested in the same mice by simply repeating the imaging procedure. Indeed, two days after the first imaging event, when the injected PD0325901 was likely to be fully cleared, a second test on the same group of mice with another MEK inhibitor, GSK1120212, also revealed successful ERK pathway inhibition (FIG. 7f-h). Thus, tKiMBI allows multiple kinase inhibitors to be tested and compared in a single animal.

### Example 3—Reporting BBB Permeability of Kinase Inhibitors by Expressing KiMBI in the Brain

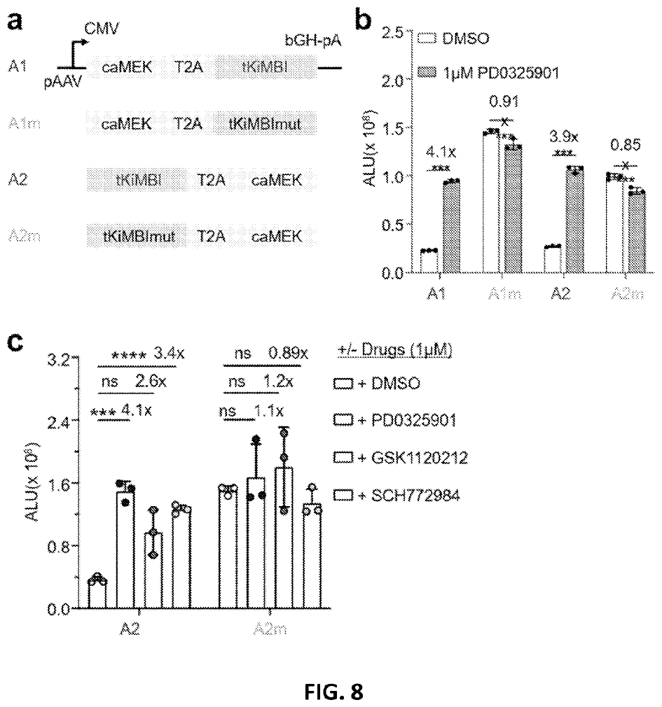

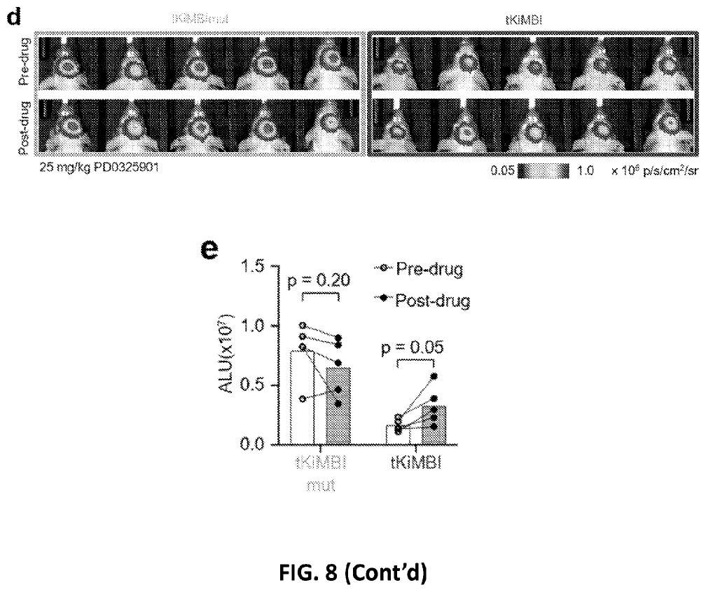

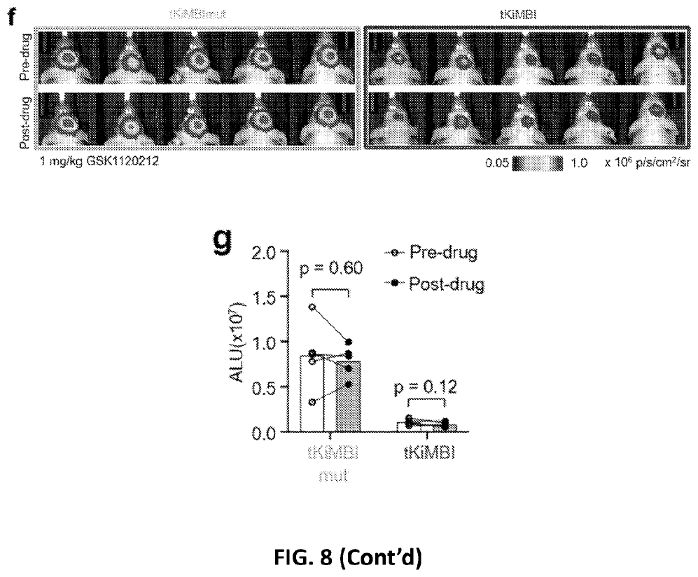

Imaging tKiMBI in the mouse brain could provide a rapid and inexpensive assay for BBB-permeance of candidate kinase inhibitors for brain tumors. To evaluate the BBB permeability of kinase inhibitors, tKiMBI was expressed in the brain using AAV. Because in normal cells ERK activity is tightly regulated and usually not hyperactive, which would limit the dynamic range of tKiMBI, AAV plasmids were constructed to introduce both tKiMBI and caMEK into cells in the brain. Since the stoichiometric ratio between caMEK and tKiMBIs may affect tKiMBI performance, bicistronic reporter genes with opposite arrangements, caMEK-T2A-tKiMBI (A1) and tKiMBI-T2A-caMEK (A2), were cloned into an AAV packaging plasmid for comparison in cell-based bioluminescence assays (FIG. 8a). When transiently transfected in HEK293A cells, both A1 and A2 reporter genes produced approximately 4-fold induction of tKiMBI signal upon PD0325901 treatment (FIG. 8b). In further testing with different kinase inhibitors, tKiMBI responses in A2-transfected cells demonstrated similar responses as in the tKiMBI-expressing U87-EGFRvIII stable cell line (FIG. 8c compared to FIG. 6c).

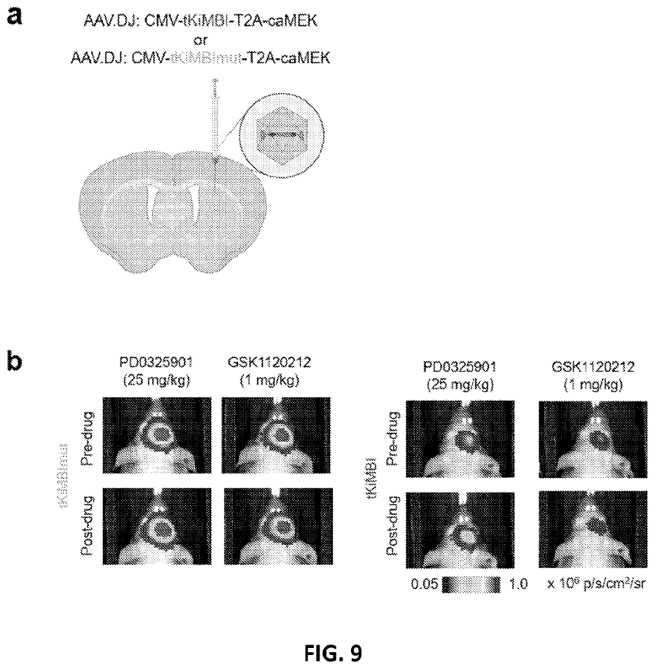

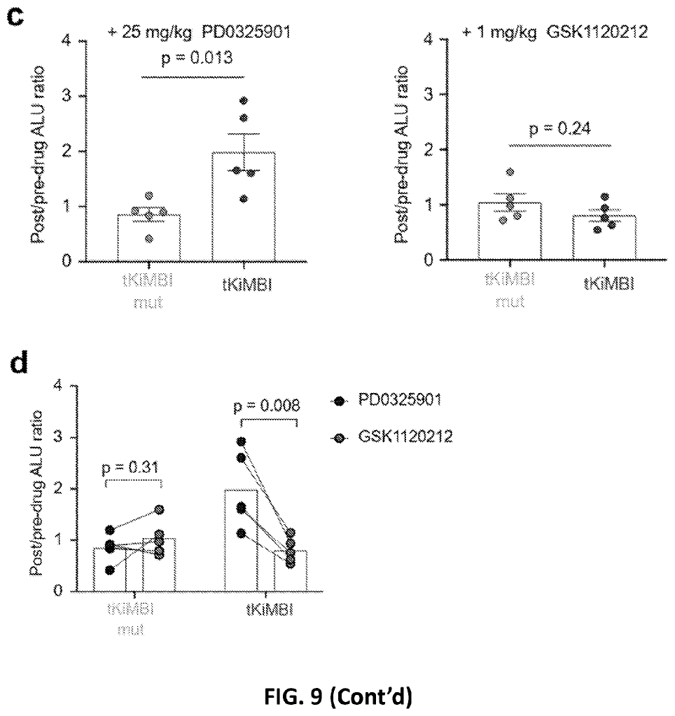

The recent development of cephalofurimazine (CFz), a NanoLuc substrate with improved BBB permeability, substantially improves the sensitivity of bioluminescence imaging in the brain. Using CFz, tested was whether tKiMBI could report the BBB-permeance of ERK pathway inhibitors in living mice. tKiMBI-T2A-caMEK or the negative control tKiMBIₘᵤₜ-T2A-caMEK were expressed in mouse striatum by AAV transduction (FIG. 9a). Treatment with 25 mg/kg PD0325901, previously characterized to be BBB-permeant¹², robustly induced bioluminescence of tKiMBI, but not of tKiMBIₘᵤₜ (FIG. 9b,c and FIG. 8d,e). However, when mice were treated with MEK inhibitor GSK1120212, previously found to have poor BBB permeation¹³, there was no signal increase at 1 mg/kg (FIG. 9b,c and FIG. 8f,g), a dose that is however effective against extracranial tumors in mice^{14,15}. These results confirm that tKIMBI induction in a target tissue requires perfusion of ERK pathway inhibitors into that tissue. They also suggest that tKIMBI, when co-expressed in the healthy brain with caMEK, can be used to assess BBB permeation by ERK pathway inhibitors.

### Example 4—Non-Invasive Imaging of ERK Inhibition in a Brain Tumor Xenograft Model

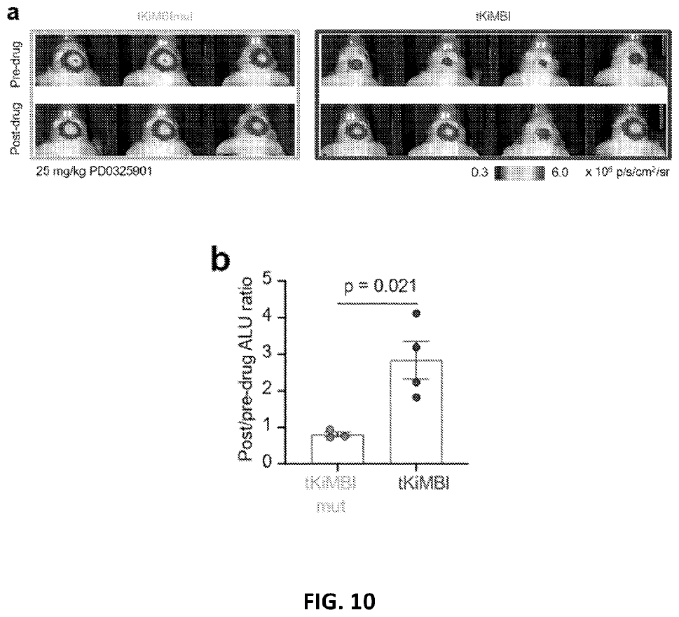

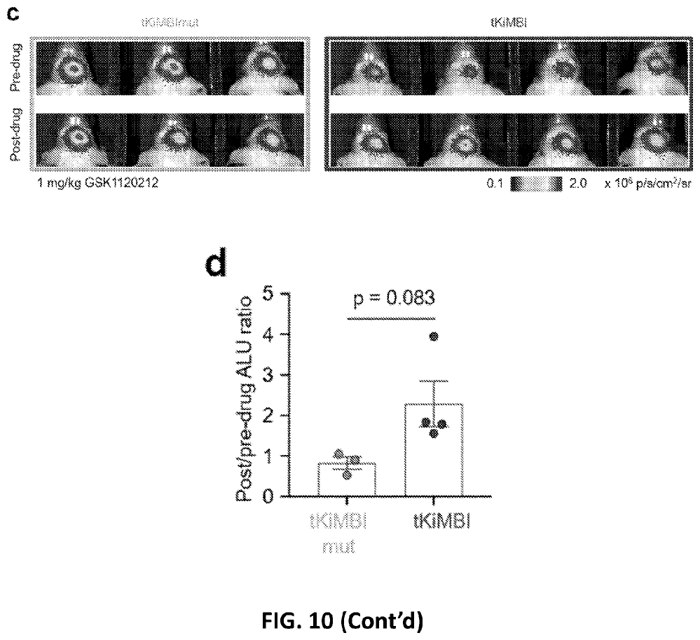

Tested next was whether KiMBI can report ERK inhibition in the brain tumor xenograft model. 3×10⁴ tKiMBI- or tKiMBIₘᵤₜ-expressing U87-EGFRvIII cells were implanted into the striatum and tKiMBI induction by the BBB-permeant MEK inhibitor PD0325901 was assessed, by using the BBB-permeable NanoLuc substrate CFz. A ˜200% increase in tKiMBI signal was observed after PD0325901 administration, compared to a ˜10% decrease in tKiMBImut signal (FIG. 10a,b). This result thus confirms that tKiMBI can report ERK pathway inhibition within tumor cells in the brain.

U87-EGFRvIII xenografts are known to disrupt the BBB¹⁶. When the BBB-impermeant GSK1120212 (1 mg/kg i.p.) was administered to the same group of mice, a positive response was also observed, with ˜150% higher tKiMBI signal. Given the earlier finding described above that signals from tKiMBI expressed by AAV were not induced by GSK1120212, this result indicates that the BBB was indeed compromised in the U87-EGFRvIII xenografts (FIG. 10c,d).

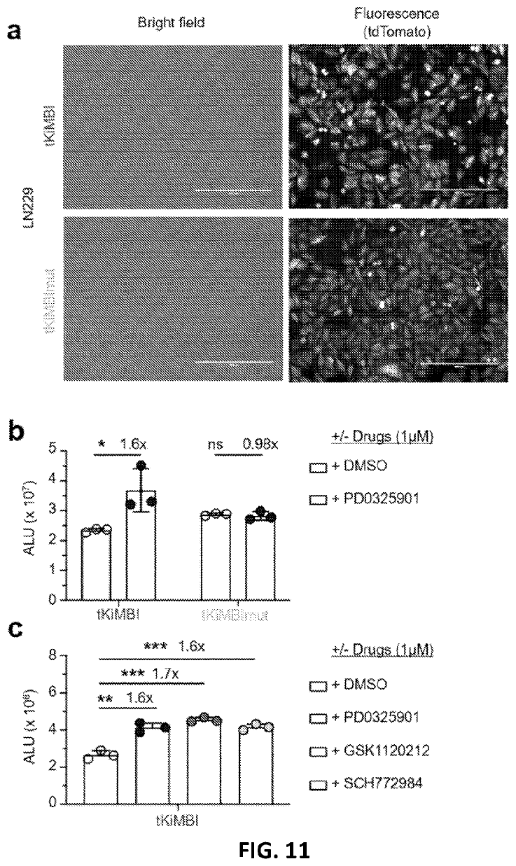

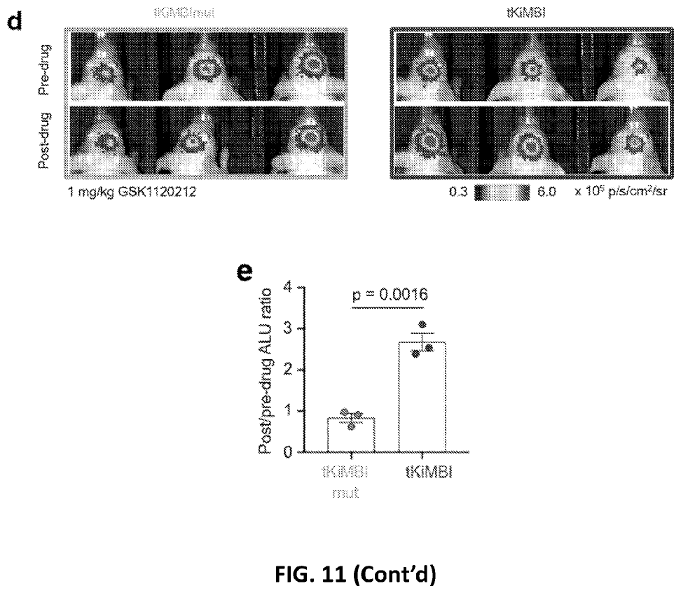

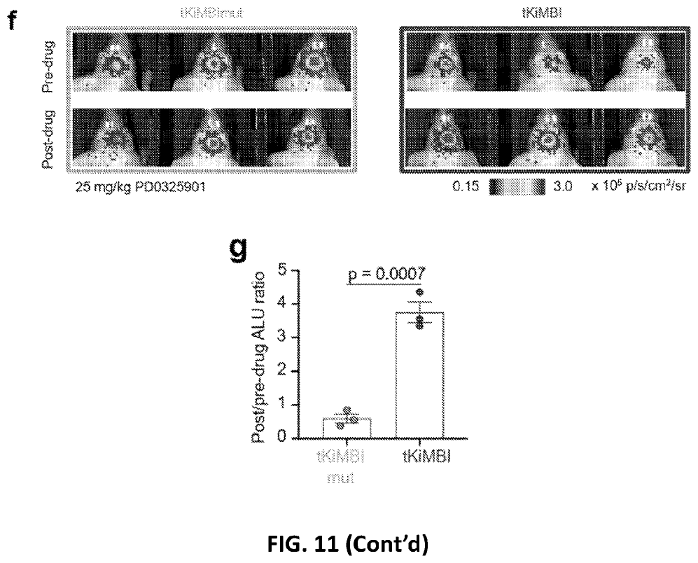

To study whether the BBB is compromised in another glioma line, LN-229 human glioblastoma cells were stably transduced with tKiMBI or tKiMBIₘᵤₜ (FIG. 11a). In culture, the tKiMBI signal from these cells only exhibited a roughly 1.6-fold signal increase after treatment with MEK-ERK targeting inhibitors, which suggested lower ERK activity levels than in U87-EGFRvIII cells (FIG. 11b,c). 3×10⁴ LN-229 cells expressing tKiMBI or tKiMBIₘᵤₜ were implanted into the striatum, and KiMBI assays were performed one week later. Intriguingly, in vivo, there was a >3-fold increase in signal from tKIMBI after PD0325901, which was even higher than observed in U87-EGFRvIII xenografts (FIG. 11d,e). This suggests that when implanted in mouse brain, LN-229 cells robustly activate the ERK pathway. Finally, GSK1120212 was observed to also induce tKIMBI signals in xenografted LN-229 cells, suggesting that these xenografts also result in a leaky BBB (FIG. 11f,g). These results thus demonstrate that tKiMBI and a BBB-impermeant kinase inhibitor can be used to ascertain BBB integrity in mouse brain tumor models.

### Example 5—Non-Invasive Assessment of Kinase Inhibitor Persistence in the Brain

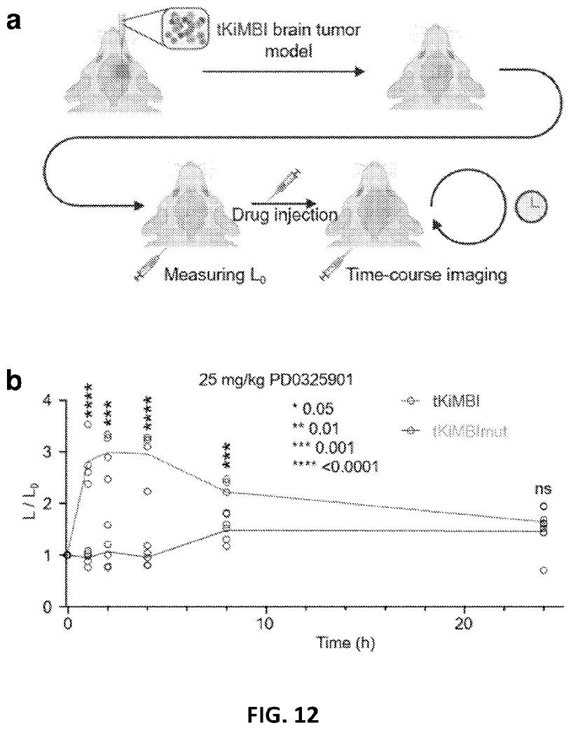

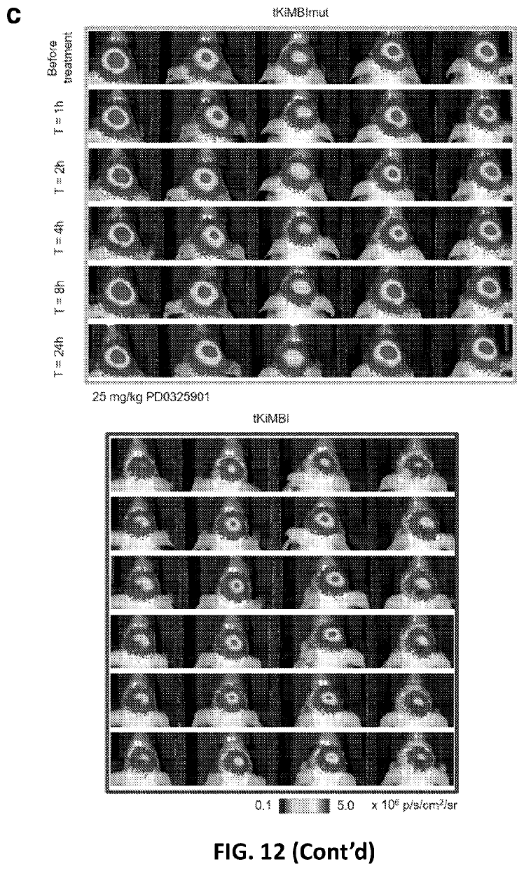

A non-invasive method with the potential for longitudinal studying of drug pharmacokinetics and pharmacodynamics should allow rapid dose and schedule optimization for drug development. To evaluate whether the KiMBI reporter can report the dynamics of drug candidates in vivo, imaging of the U87-EGFRvIII reporter cells was carried out in a time series after PD0325901 administration (25 mg/kg i.p., FIG. 12). In response to PD0325901, the bioluminescence signal in tKiMBI-expressing cells rapidly rose to a plateau within 1 h of treatment and remained high until about 4 h. Signal then declined to about half at around 8 hrs post-treatment, and backed to baseline on the second day. In contrast, the control cells only exhibited an approximate 1.4-fold signal increase after 24 h, which could be explained by continued proliferation of the engrafted tumor cells.

To summarize, described herein is a kinase-modulated bioluminescent indicator (KiMBI) that responds to kinase inhibition in vivo with increased light emission. An ERK-modulated KiMBI expressed in tumor xenografts in living mice specifically reports administration of ERK pathway inhibitors, and allows kinetic measurements of target engagement in the same subjects overtime. In addition, KiMBI expressed in the brain discriminates between brain-permeant and -impermeant inhibitors. The KiMBI method thus enables rapid identification of kinase inhibitors with desirable pharmacokinetic and pharmacodynamic properties in vivo.

Kinase indicators based on fluorescent proteins are widely used for live-cell experiments, where their high photon flux enables visualization of kinase activity with spatial and temporal resolutions on the micron and millisecond scales respectively¹⁷. However, as the excitation light required for fluorescence imaging is difficult to deliver uniformly through tissue, fluorescent indicators can only be used in living mammals by inserting lenses of fibers to access specific sites of interest¹⁸. This is invasive, severely restricts the field of view, and adds to experimental variability between animals. In contrast to fluorescence, bioluminescence from luciferase proteins is suitable for non-invasive imaging at millimeter and second scales. The absence of excitation light and autoluminescence removes background signal, allowing the relatively low photonic output of luciferase reporters to be detected from even deep locations in the body, with sensitivity limited by camera dark current and read noise^{19,20}.

The present study demonstrates the advantages of genetically targeted non-invasive imaging for preclinical evaluation of kinase inhibitors. Traditional methods for characterizing drugs in animal models involve time- and labor-consuming tissue collection and subsequent biochemical analysis. Due to experimental variation and the ability of each animal to contribute only one data point, such experiments require large numbers of animals. In contrast, the KiMBI imaging procedure is rapid and inexpensive, involving only injection of the luciferase substrate, anesthesia, and imaging. KiMBI imaging also allows each animal to provide information on the effects of one drug at multiple concentrations and timepoints. This efficiency is evidenced by the study herein on subcutaneous tumor xenografts (FIG. 6). Although there was large individual variance in tumor sizes (FIG. 6b), which might introduce variability in kinase activity assays after dissection, the longitudinal nature of KiMBI imaging allowed assessment of the relative signal change within the same mouse (FIG. 6e,h). In addition, KiMBI imaging allows the same group of subjects to report on multiple drugs (FIG. 6c,f), further improving efficiency.

This study focused on PD0325901 (mirdametinib) and GSK1120212 (trametinib), because both MEK inhibitors have been approved by FDA as anti-cancer drugs and their BBB permeability have been well studied¹³. In a study where mouse brains were extracted for histochemical analysis after drug administration, PD0325901 was shown to inhibit MEK activity in the brain and thus block ERK phosphorylation, while GSK1120212 did not show any effects¹². In addition, a pharmacokinetics and pharmacodynamic analysis of GSK1120212 in harvested brain tissues revealed no detectable GSK1120212 or ERK inhibition in the brains of drug-injected mice¹⁴. The difference between the BBB permeability of these two inhibitors is consistent with the observation herein from the AAV-infected mice brain, where PD032901 but not GSK1120212 was able to induce a signal increase in KiMBI.

Tumor growth in the brain often results in a compromised BBB¹⁶. Indeed, the results herein with U87-EGFRvIII and LN-229 xenografts in the brain suggest BBB disruption, since drugs known to be BBB-impermeable such as GSK1120212 still activated ERK KiMBI. However, drugs with good BBB permeability are still required for maximally effective treatment of glioblastoma, as low-grade gliomas and a portion of progressive glioblastomas exhibit an intact BBB²¹. In short, mouse brain tumors may exhibit more BBB disruption than the corresponding human tumors they are intended to model. Evaluating candidate kinase inhibitors by measuring inhibition of brain tumor growth in mice, also possible using noninvasive luciferase imaging, may thus produce false-positive results. Thus, the evaluation of tumor growth cannot substitute for an independent assessment of BBB permeability of kinase inhibitors. KiMBI expressed in the brain by viral transduction provides a longitudinal non-invasive measure of BBB permeability for a drug, without the need for brain tissue dissection and biochemical assays.

NanoLuc-based KiMBIs differ from earlier kinase reporters based on firefly luciferase (FLuc) in two important ways. First, Nanoluc-RFP fusions with the substrate cephalofurimazine produce an order of magnitude more light in the brain than FLuc with either D-luciferin or cycLuc substrate. The higher achievable brightness of NanoLuc-based reporters are expected to allow drug effects to be detected at lower levels of reporter expression or with fewer cells than FLuc-based reporters, which is less likely to interfere with normal biological processes. Second, because FLuc is ATP-dependent, it has the potential to generate spurious signals if kinase inhibition, or off-target drug activity, alters cellular energy states. Indeed, FLuc alone serves as a reporter of ATP levels in cells. In contrast, as NanoLuc is ATP-independent, KiMBIs can report kinase inhibition independently of ATP.

It is believed that no FLuc-based reporter for the Ras-Raf-MEK-ERK kinase pathway of any design has previously been reported. This is remarkable considering that proteins in this pathway are major drug targets in cancer, and that FLuc-based reporters have been reported for Akt, c-Met, EGFR, and ATM. The development of ERK KiMBI thus also fills a previously unmet need for non-invasive reporting of Ras-ERK pathway activity in mammals.

The modular design of KiMBIs facilitates generalization to other kinase targets of therapeutic interest. The engineering of PKA and ERK KiMBIs demonstrated in the preceding examples shows that obtaining a reporter that responds positively to kinase inhibition may require different ordering of elements (LargeBiT, SmallBiT, substrate, and PBP) for different kinases. Presumably this is due to the different orientations that phosphosubstrates can interact with different PBPs relative to PBP termini (FHA for PKA, WW for ERK). With the benefit of the present disclosure, it is expected that other PBPs such as 14-3-3 or SH2 domains could replace the WW domain in ERK KiMBI to develop indicators for a broader range of kinases. As a simple screen of possible topologies with PBP and/or substrate sequences linking LgBiT and SmBiT fragments was sufficient to obtain KiMBIs for PKA and ERK, it is expected that KiMBIs that use other PBPs can also be obtained in this manner.

By reporting target inhibition in tissues of interest, KiMBI reporters will facilitate the acceleration of kinase inhibitor discovery and provide a link between kinase inhibition and tumor responses. With KiMBI revealing whether a kinase inhibitor engages its target in vivo in a rapid and inexpensive manner, multiple candidates can be screened and their administered doses optimized. Another potential use of KiMBI would be to relate the extent and duration of kinase inhibition to tumor shrinkage, which could establish or refute proposed functions for the kinase in tumor growth.

### Methods

Chemicals. Drugs used in the paper include ERK inhibitor Vx-Ile (Selleck Chemicals), MEK inhibitors U0126 (Tocris), PD0325901 (Selleck Chemicals, ApexBio), GSK1120212 (Selleck Chemicals, ApexBio), and SCH772984 (Selleck Chemicals), JNK inhibitor SP600125 (Selleck Chemicals), and p38 inhibitor PD169316 (ApexBio). Antibiotics used in cell culture include Blasticidin (Invivogen), Geneticin (Invitrogen), penicillin/streptomycin (Gemini Bio), ciprofloxacin (Sigma-Aldrich), piperacillin (Sigma-Aldrich).

Molecular Cloning. DNA primers for molecular cloning were synthesized by Intergrated DNA Technologies. WW domain was PCR amplified from pcDNA3-ERK-SPARK, a gift from Xiaokun Shu (Addgene plasmid #106921). Molecular cloning was typically carried out using infusion HD cloning kit (Takara Bio) with ˜20 overlapping base primers and the PrimeSTAR HS DNA Polymerase (Clontech) or the Phusion Flash High-Fidelity PCR Master Mix (Thermo Scientific). Constructed plasmids were verified using Sanger sequencing by Elim Biopharm. The KiMBI constructs were assembled in pcDNA3.1 vector (Addgene) behind the CAG promoter. The best KiMBI sensors (bKiMBI, oKiMBI, and tKiMBI) were then cloned into the pLL3.7m lentiviral vector, or the AAV packaging vector under the CMV promoter.

Cell lines. HEK293A cells (Invitrogen) were cultured at 37° C. with 5% CO₂ in Dulbecco's Modified Eagle's Medium (DMEM) supplemented with 10% fetal bovine serum (FBS), 2 mM L-glutamate, 100 U ml⁻¹ penicillin and 100 μg ml⁻¹ streptomycin. U87 (ATCC, No. HTB-14) and U87-EGFRvIII (previously developed in Lin lab by pcDNA3-EGFRvIII transfection and Geneticin selection) cells were cultured at 37° C. with 5% CO₂ in DMEM supplemented with 10% bovine calf serum (BCS), 2 mM L-glutamate, 100 U ml⁻¹ penicillin, 100 μg ml⁻¹ streptomycin, 0.8 mg/mL Geneticin, 10 μg ml⁻¹ Ciprofloxacin, 10 μg ml⁻¹ Piperacillin. SK-OV-3 cells (ATCC, No. HTB-77) were cultured at 37° C. with 5% CO₂ in Roswell Park Memorial Institute (RPMI) 1640 medium supplemented with 10% FBS, 2 mM L-glutamate, 100 U ml⁻¹ penicillin and 100 μg ml⁻¹ streptomycin. LN229 cells (ATCC, No. CRL-2611) were cultured at 37° C. with 5% CO₂ in DMEM supplemented with 5% FBS, 2 mM L-glutamate, 100 U ml⁻¹ penicillin and 100 μg ml⁻¹ streptomycin.

Virus packaging. For lentivirus packaging, the pLL3.7m plasmids were purified by PureLink Expi Endotoxin-free Maxi plasmid Purification kit (Invitrogen), and then HEK293T (ATCC, No. CRL-3216) cells at ˜70% confluency were transfected with psPAX2, pMD2.G, and pLL3.7m plasmids using CalPhos mammalian transfection kit (Takara Bio). Two days following transfection, viral supernatant was filtered with a 0.45 μm PES filter before using to infect target cells. For AAV packaging, pAAV-CMV-tKiMBI-T2A-caMEK-bGH_pA plasmids were purified by PureLink Expi Endotoxin-free Maxi plasmid Purification kit (Invitrogen), and then were sent to Stanford Gene Vector and Virus Core (GVVC) to produce and titer AAV.DJ vectors.

Generation of KiMBI-expressing stable tumor cell lines. U87-EGFRvIII and LN229 cells were lentiviral transduced with pLL3.7m-CMV-KiMBI-T2A-AkaLuc-P2A-Blasticidin. Then, 72 h after transfection, cells were dissociated with trypsin and resuspended in DMEM and then changed to PBS. Suspended cells were sorted for the population with medium fluorescence level (20-40%) on a fluorescence-activated cell sorter (FACS), the FACSJazz (BD Biosciences) at Stanford Shared FACS Facility. After cell sorting, the KiMBI-expressing cells were maintained and passaged in corresponding culture medium supplemented with blasticidin (6 μg/mL for both cell lines) to select for stable polyclonal cell lines. Flow cytometry data were analyzed on FlowJo software. The stable KiMBI expression was validated by examination of tdTomato fluorescence in tKiMBI (and its variants) using an EVOS FL Autoimaging system (Thermo Scientific).

In vitro cell-based bioluminescent assay. Cells were seeded into 96-well Lumitrac plate (Greiner Bio-One) at a density of 1.5-2×10⁴ cells per well. After 24 h, cells were transiently transfected with KiMBI-expressing plasmids (5-10 ng per well) with or without caMEK-expressing plasmids (50 ng per well, empty pcDNA3.1 as negative control to keep total transfected DNA amount consistent) using lipofectamine 3000 (Invitrogen) following manufacturer's instructions. For the stable polyclonal KiMBI-expressing cell liens, this lipofection step was skipped. At 24 h after transfection, the cells were treated with drugs at desired concentration in opti-MEM reduced-serum medium (Thermo Fisher) for 1-1.5 hours. Then, for assaying luminescence, Nano-Glo live cell assay system (Promega) was used following manufacturer's instructions. Time-lapse live luminescence was recorded on Safire-2 microplate reader (Tecan) with 100 ms integration and one read per min for 20 min. Luminescence spectrum was measured on a Varioskan LUX multimode microplate reader (Thermo Scientific) using the spectral scanning protocol with 1 s integration for each wavelength at 400-680 nm (1 nm per data point).

General procedures of kinase inhibitor injection and in vivo bioluminescence imaging. For drug administration in mice, kinase inhibitors (PD0325901 and GSK1120212) were dissolved in an injectable formulation containing 5% DMSO, 40% PEG-300, 5% Tween-80 (v/v) in water, and each mouse received a dose (25 mg/kg for PD0325901, 1 or 3 mg/kg for GSK11230212, in a volume of 150 μL) via i.p. injection. For KiMBI (NanoLuc-based bioluminescent reporter) imaging, mice were i.p. injected with either 1.3 μmol (0.56 mg) of fluorofurimazine (FFz) or cephalofurimazine (CFz) in 150 μl of injectable formulation containing 6 mg poloxamer-407 (P-407) in Dulbecco Phosphate Buffered Saline (DPBS, without Ca²⁺ or Mg²⁺, no. 21-031-CV, Corning) for imaging. Immediately after luciferin administration, mice were anesthetized using isoflurane, and images were acquired in the Ami HT optical imaging system every 1 min for 10 min, with an air flow containing 1-2% isoflurane for stable anesthesia. Imager settings were: emission filter, open; field of view, 25 cm; f-stop 1.2; binning, 2×2 and exposure time, 30 s. Images were analyzed in Aura 4.0 in vivo imaging software. The above-mentioned conditions and settings were for most in vivo studies unless otherwise stated in the specific procedures.

Molecular imaging of ERK inhibition in subcutaneously implanted cells. For imaging ERK inhibition in subcutaneously implanted cells, U87-EGFRvIII cells stably expressing KiMBIs were dissociated with trypsin and resuspended in DMEM and then changed to PBS with a density of 2×10⁷ cells/mL. 10⁶ cells were resuspended in 100 μl FBS-free Opti-MEM containing 50% Matrigel matrix (Corning). First, 8- to 10-week-old male nude mice (strain J:NU No.007850, Jackson Laboratories) were anesthetized using isoflurane. Cells were subcutaneously injected into the thoracolumbar regions. Mice were recovered on heat pads for 30 min while cells were allowed to settle. One week after cell implantation, for visualizing tumor growth, mice were i.p. injected with 1.5 μmol (0.5 mg) of AkaLumine-HCl (Sigma-Aldrich) in 100 μl 0.9% NaCl for imaging (binning, 1×1; exposure time, 10 s). To visualize ERK inhibition, tumor-bearing mice were i.p. injected with FFz (0.9 μmol, with 6 mg P-407) for imaging. Two hours after the first imaging, kinase inhibitors in the injectable formulation were i.p. injected, and a second imaging was carried out 2 hours post drug administration following the same procedures as the first imaging.

Molecular imaging of ERK inhibition in KIMBI-encoded AAV infected mouse brain. Under sterile conditions, the 9-week-old male J:NU mice were anesthetized with isofluorane and secured in a stereotaxic frame (RWD Life Science, Shenzhen, China), and a hole of the size of the needle was drilled through the skull. A Hamilton syringe with a 33-gauge needle was inserted at 0.5 mm dorsal and 2.0 mm lateral to the bregma to a depth of 3.1 mm, and after a 2 min wait the needle was pulled back 0.3 mm to allow space for the virus solution. 1.5 μL of AAV vector solution (2×10¹² viral genomic titers/mL) was injected at a speed of 0.15 μL/min using a syringe pump (KD Scientific, Holliston, MA). The needle was left in place for 3 min after each injection to minimize upward flow of viral solution after raising the needle. To visualize ERK inhibition, four weeks after AAV infection, mice were i.p. injected with CFz (1.2 μmol, with 6 mg P-407) for imaging (binning, 4×4; exposure time, 60 s). Two hours after the first imaging, kinase inhibitors in the injectable formulation were i.p. injected, and a second imaging was carried out 2 hours post drug administration following the same procedures as the first imaging.

Molecular imaging of ERK inhibition in intracranially implanted cells. The stereotaxic injection on the 7- to 9-week-old male J:NU mice were similar to the afore-mentioned AAV infection surgery. A Hamilton syringe with a 26-gauge needle was inserted 0.62 mm dorsal and 1.75 mm lateral to the bregma to a depth of 3.5 mm, and after a 2 min wait the needle was pulled back 0.5 mm to allow space for the cell suspension. Following this, 3×10⁴ KiMBI-expressing U87-EGFRvIII or LN-229 stable cells in 1.5 μl of PBS were injected at an injection speed of 0.3 μL/min using the syringe pump. To visualize ERK inhibition in U87-EGFRvIII cell implants, 5 days after cell implantation, tumor-bearing mice were i.p. injected with CFz (0.4 μmol, with 1.8 mg P-407) for imaging. Two hours after the first imaging, kinase inhibitors in the injectable formulation were i.p. injected, and a second imaging was carried out 2 hours post drug administration following the same procedures as the first imaging. To visualize ERK inhibition in LN-229 cell implants, imaging was done 7 days after cell implantation, and 1.05 or 0.7 μmol CFz with 5 or 3.3 mg P-407 was i.p. injected to each mouse for imaging (binning 4×4; exposure time, 30 s).

Time-lapse imaging of ERK inhibition in intracranially implanted cells. One week after implantation of 10⁵ KiMBI-expressing U87-EGFRvIII stable cells, the tumor-bearing mice were i.p. injected with CFz (0.2 μmol, with 0.8 mg P-407) for the initial imaging to establish the signal baseline before drug administration. Two hours after the first imaging, PD0325901 (25 mg/kg) in the injectable formulation were i.p. injected, and the subsequent imaging were carried out at 1, 2, 4, 8, 24 hours post drug administration following the same procedures as the initial imaging.

Statistics. Student's t-test, one-way analysis of variance (ANOVA) with Tukey's posthoc test were performed in GraphPad Prism 9.

### REFERENCES FOR INTRODUCTION AND EXAMPLES 1-5

1. Cohen, P., Cross, D. & Jänne, P. A. Kinase drug discovery 20 years after imatinib: progress and future directions. Nat Rev Drug Discov 20, 551-569 (2021).
2. Pearson, J. R. D. & Regad, T. Targeting cellular pathways in glioblastoma multiforme. Signal Transduct Target Ther 2, 17040 (2017).
3. Zhang, D. et al. Drug Concentration Asymmetry in Tissues and Plasma for Small Molecule-Related Therapeutic Modalities. Drug Metab Dispos 47, 1122-1135 (2019).
4. de Gooijer, M. C. et al. The impact of β-glycoprotein and breast cancer resistance protein on the brain pharmacokinetics and pharmacodynamics of a panel of MEK inhibitors. Int J Cancer 142, 381-391 (2018).
5. Di, L., Kerns, E. H. & Carter, G. T. Strategies to assess blood-brain barrier penetration. Expert Opin Drug Discov 3, 677-687 (2008).
6. Heffron, T. P. Small Molecule Kinase Inhibitors for the Treatment of Brain Cancer. J Med Chem 59, 10030-10066 (2016).
7. Gonzalez, F. A., Raden, D. L. & Davis, R. J. Identification of substrate recognition determinants for human ERK1 and ERK2 protein kinases. J Biol Chem 266, 22159-22163 (1991).
8. Lu, P. J., Zhou, X. Z., Shen, M. & Lu, K. P. Function of WW domains as phosphoserine- or phosphothreonine-binding modules. Science 283, 1325-1328 (1999).
9. Verdecia, M. A., Bowman, M. E., Lu, K. P., Hunter, T. & Noel, J. P. Structural basis for phosphoserine-proline recognition by group IV WW domains. Nat Struct Biol 7, 639-643 (2000).
10. Chu, J. et al. A bright cyan-excitable orange fluorescent protein facilitates dual-emission microscopy and enhances bioluminescence imaging in vivo. Nat Biotechnol 34, 760-767 (2016).
11. Su, Y. et al. Novel NanoLuc substrates enable bright two-population bioluminescence imaging in animals. Nat Methods 17, 852-860 (2020).
12. Papale, A. et al. Impairment of cocaine-mediated behaviours in mice by clinically relevant Ras-ERK inhibitors. Elife 5, e17111 (2016).
13. Vaidhyanathan, S., Mittapalli, R. K., Sarkaria, J. N. & Elmquist, W. F. Factors influencing the CNS distribution of a novel MEK-1/2 inhibitor: implications for combination therapy for melanoma brain metastases. Drug Metab Dispos 42, 1292-1300 (2014).
14. Gilmartin, A. G. et al. GSK1120212 (JTP-74057) is an inhibitor of MEK activity and activation with favorable pharmacokinetic properties for sustained in vivo pathway inhibition. Clin Cancer Res 17, 989-1000 (2011).
15. Yamaguchi, T., Kakefuda, R., Tajima, N., Sowa, Y. & Sakai, T. Antitumor activities of JTP-74057 (GSK1120212), a novel MEK1/2 inhibitor, on colorectal cancer cell lines in vitro and in vivo. Int J Oncol 39, 23-31 (2011).
16. Arvanitis, C. D., Ferraro, G. B. & Jain, R. K. The blood-brain barrier and blood-tumour barrier in brain tumours and metastases. Nat Rev Cancer 20, 26-41 (2020).
17. Greenwald, E. C., Mehta, S. & Zhang, J. Genetically Encoded Fluorescent Biosensors Illuminate the Spatiotemporal Regulation of Signaling Networks. Chem Rev 118, 11707-11794 (2018).
18. Zhang, J. F. et al. An ultrasensitive biosensor for high-resolution kinase activity imaging in awake mice. Nat Chem Biol 17, 39-46 (2021).
19. Liu, S., Su, Y., Lin, M. Z. & Ronald, J. A. Brightening up Biology: Advances in Luciferase Systems for in Vivo Imaging. ACS Chem Biol 16, 2707-2718 (2021).
20. Rice, B. W., Cable, M. D. & Nelson, M. B. In vivo imaging of light-emitting probes. J Biomed Opt 6, 432-440 (2001).
21. Leten, C., Struys, T., Dresselaers, T. & Himmelreich, U. In vivo and ex vivo assessment of the blood brain barrier integrity in different glioblastoma animal models. J Neurooncol 119, 297-306 (2014).
22. Khan, A. P., Contessa, J. N., Nyati, M. K., Ross, B. D. & Rehemtulla, A. Molecular imaging of epidermal growth factor receptor kinase activity. Anal Biochem 417, 57-64 (2011).
23. Zhang, L. et al. Molecular imaging of Akt kinase activity. Nat Med 13, 1114-1119 (2007).
24. Zhang, L., Bhojani, M. S., Ross, B. D. & Rehemtulla, A. Enhancing Akt imaging through targeted reporter expression. Mol/Imaging 7, 168-174 (2008).
25. Zhang, L. et al. Molecular imaging of c-Met tyrosine kinase activity. Anal Biochem 412, 1-8 (2011).
26. Bouquier, N. et al. AIMTOR, a BRET biosensor for live imaging, reveals subcellular mTOR signaling and dysfunctions. BMC Biol 18, 81 (2020).
27. Tomida, T., Takekawa, M. & Saito, H. Oscillation of p38 activity controls efficient pro-inflammatory gene expression. Nat Commun 6, 8350 (2015).

### Example 6—Development, Optimization, and Characterization of Akt KiMBIs

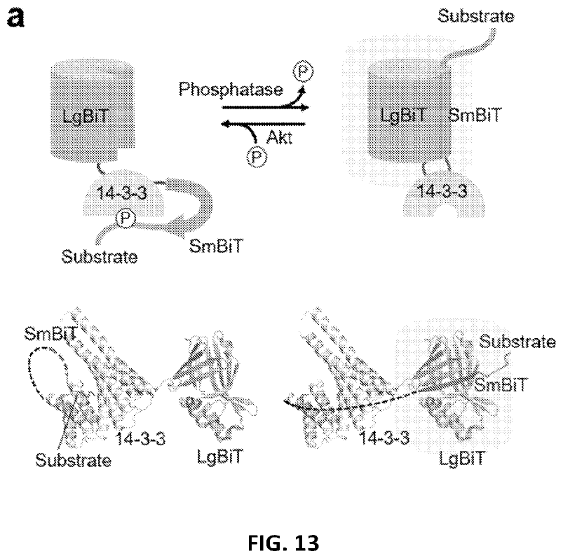

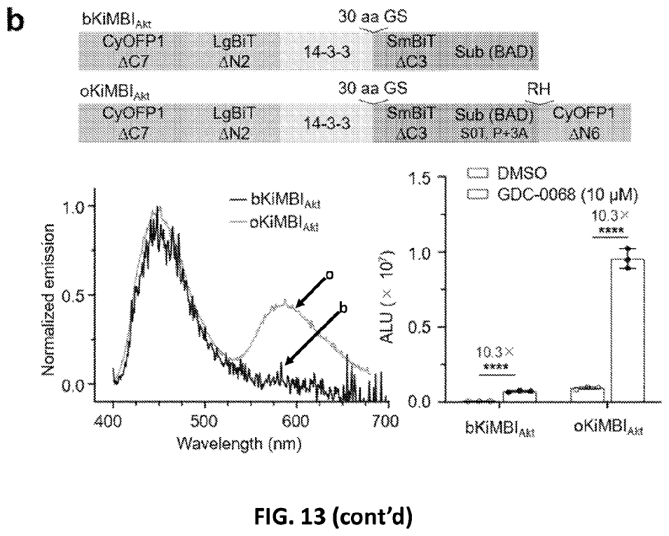

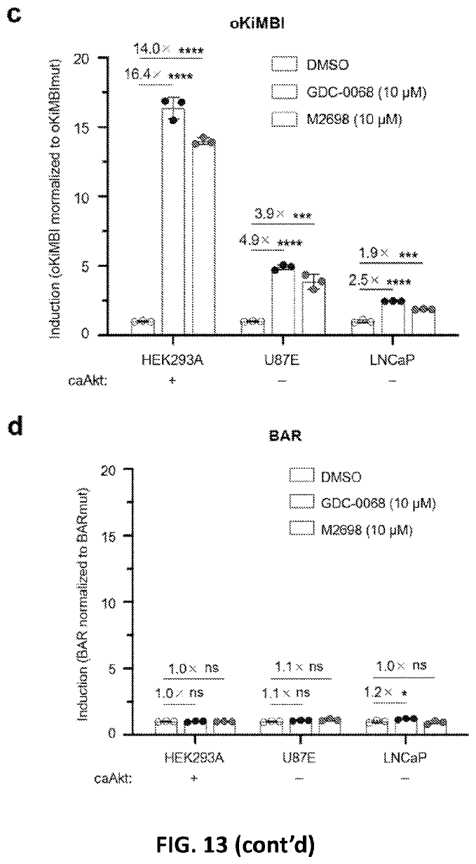

Given the importance of PI3K-Akt signaling pathway in human disease, it was aimed to engineer a kinase-modulated bioluminescent indicator for Akt inhibition (KiMBI). The design principle of KiMBIs is to engineer reporters to serve as surrogate substrates for kinases and, when dephosphorylated, generate an increase in luminescence as the result of the reconstitution of LgBiT and SmBiT, which outcompetes the phosphorylation-dependent intramolecular interaction between the kinase substrate and phosphopeptide-binding domain (PBD) (FIG. 13a).

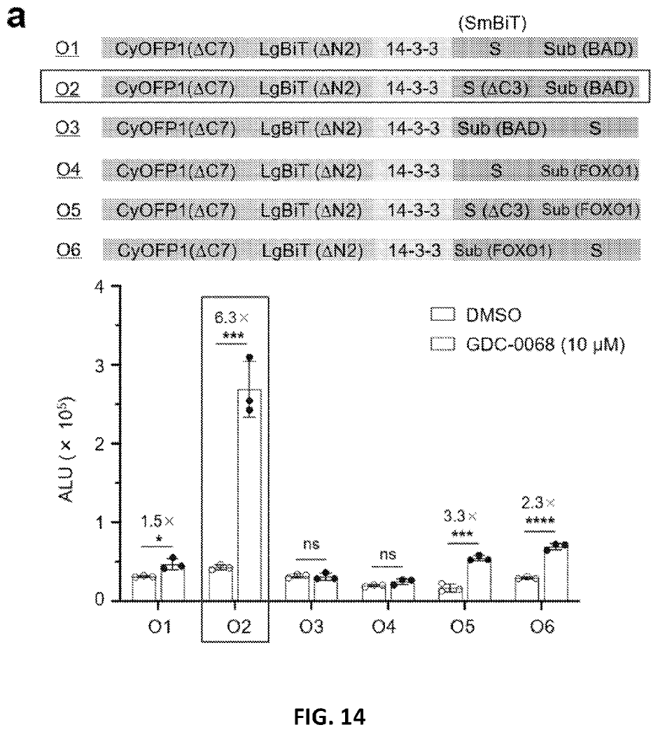

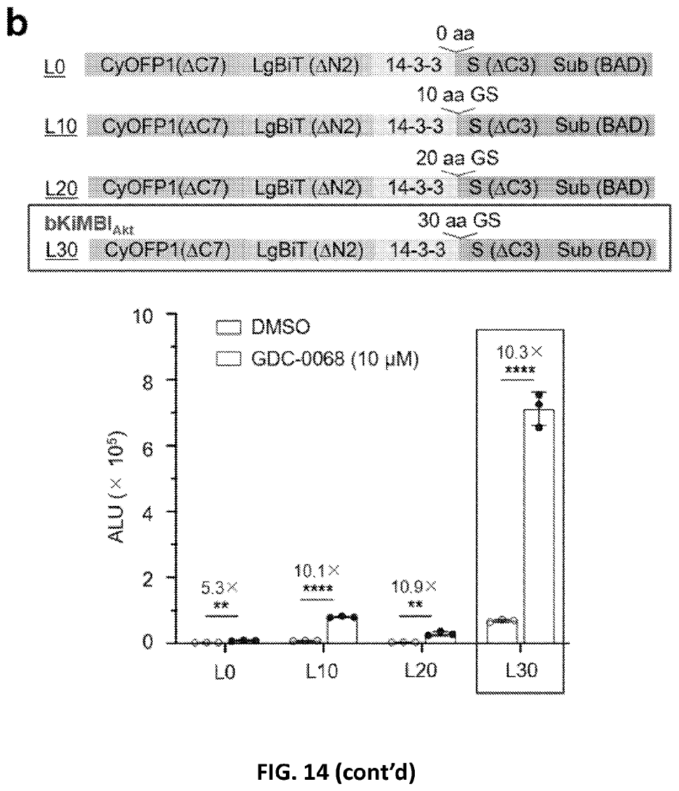

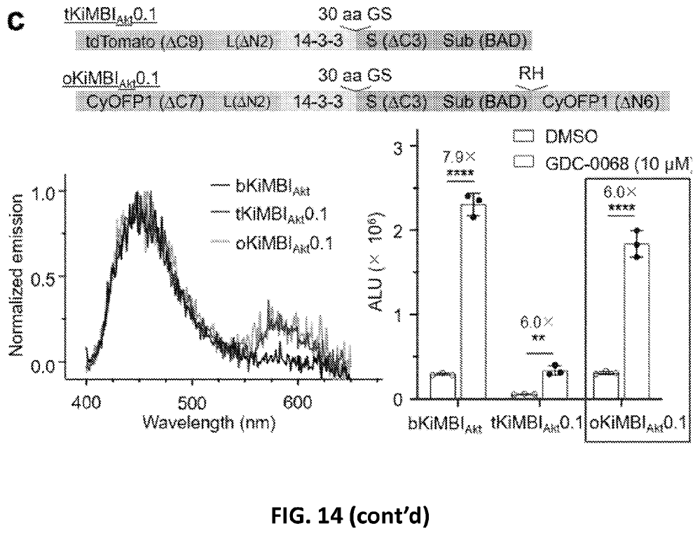

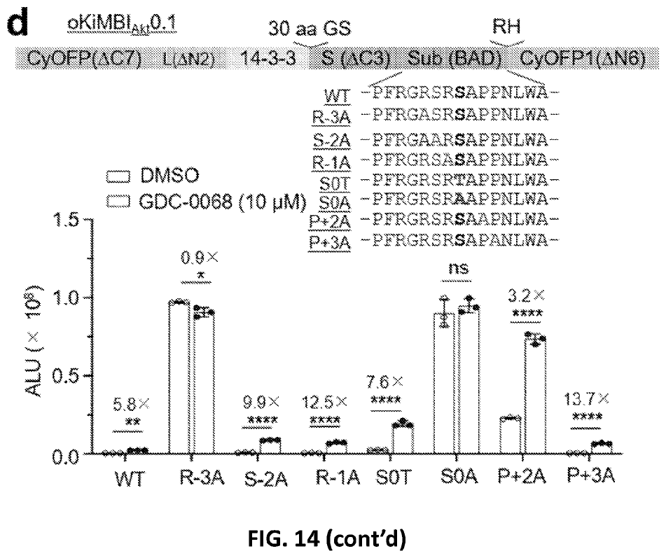

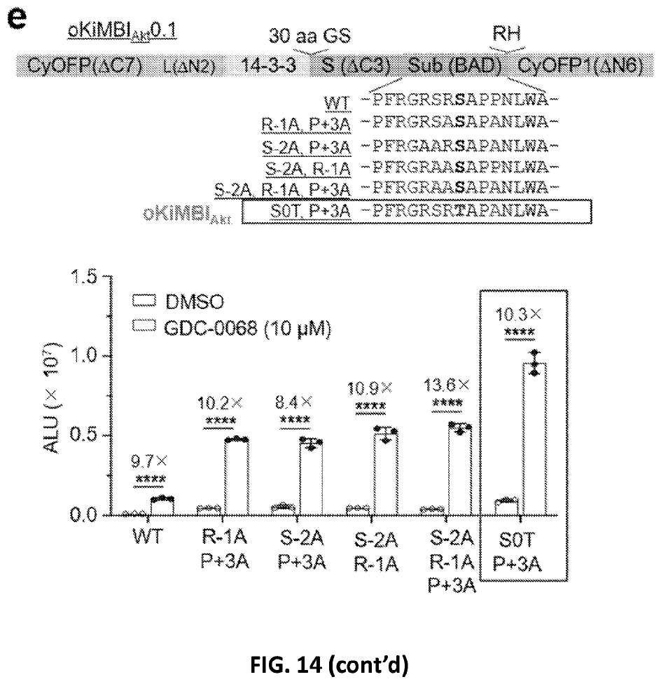

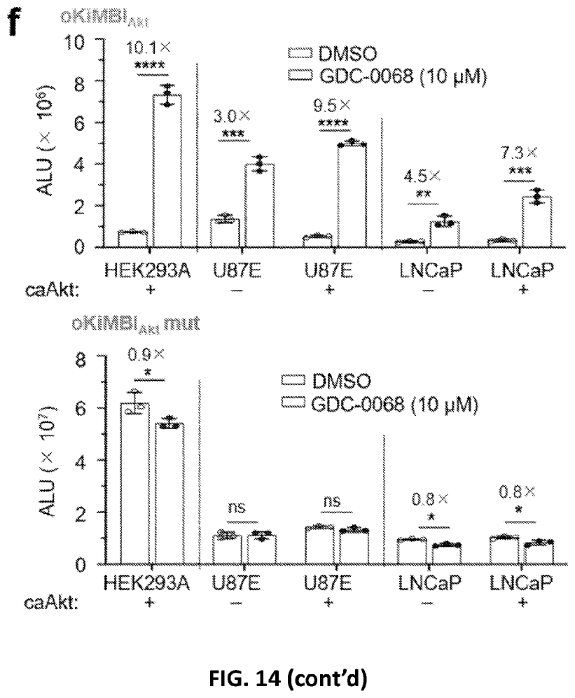

Engineered first were fusion proteins of various topologies, comprising CyOFP1, LgBiT, a phospho-binding protein 14-3-3, Akt substrates from human BAD (S99)² or human FOXO1 (T24)³, SmBiT or the SmBiT (ΔC3) variant. The fusions were tested by co-transfecting them with constitutively active Akt (caAkt) in HEK293A cells, followed by treatment of the cells with DMSO (as negative control) or the cell-permeable Akt inhibitor GDC-0068 (ipatasertib), and measurement of their luminescent signals. Among the Akt KiMBI candidates, the one with the CyOFP1-LgBiT-14-3-3-SmBiT(ΔC3)-Sub (BAD) topology exhibited 6.3-fold more luminescence when treated with GDC-0068 compared to DMSO (FIG. 14a).

To further improve response to Akt inhibitor treatment, different linker lengths were tested between 14-3-3 and SmBiT (ΔC3), ranging from 0 to 30 amino acids (aa). The variant with a 30-aa linker showed an approximately 10-fold response to GDC-0068. Despite the presence of CyOFP1 at the N-terminus, this construct showed negligible orange bioluminescence and was named Akt blue KiMBI (bKiMBI_{Akt}, FIG. 14b, FIG. 13b).

Next, to red-shift the bioluminescence spectrum of bKiMBI_{Akt} for better tissue penetration, it was aimed to improve bioluminescence resonance energy transfer (BRET) from bKiMBI_{Akt} to a fused orange or red fluorescent protein. Two approaches increased BRET: replacing the N-terminal CyOFP1 with tdTomato (yielding tKiMBI_{Akt}0.1), or introducing a second CyOFP1 fusion at its C terminus (yielding oKiMBI_{Akt}0.1) (FIG. 14c). As tKiMBI_{Akt}0.1 exhibited diminished brightness (FIG. 14c), oKiMBI_{Akt}0.1 was selected for further development.

To improve the response and brightness of oKiMBI_{Akt}0.1, the binding affinity of Akt substrate BAD and 14-3-3 was tuned. oKiMBI_{Akt}0.1 exhibited notably lower drug-induced brightness compared to its unphosphorylatable and thus maximally active control (SOA mutant). It was thus hypothesized that the tight binding of phosphorylated substrate peptide by 14-3-3 acted as a protective shield against dephosphorylation (FIG. 14d), and that a reduction in the strength of this interaction would improve drug-induced bioluminescence. Performing an alanine scanning on the substrate, several mutants were found, such as S-2A, R-1A, SOT, and P+3A, with larger relative responses and maximal brightness compared to the original oKiMBI_{Akt}0.1 (FIG. 14d). The most advantageous mutations were selected and assessed in various combinations. The double mutant S0T and P+3A showed the highest maximal brightness while retaining satisfactory inducibility, was designated as oKiMBI_{Akt} (FIG. 14e, FIG. 13b) or Akt oKiMBI.

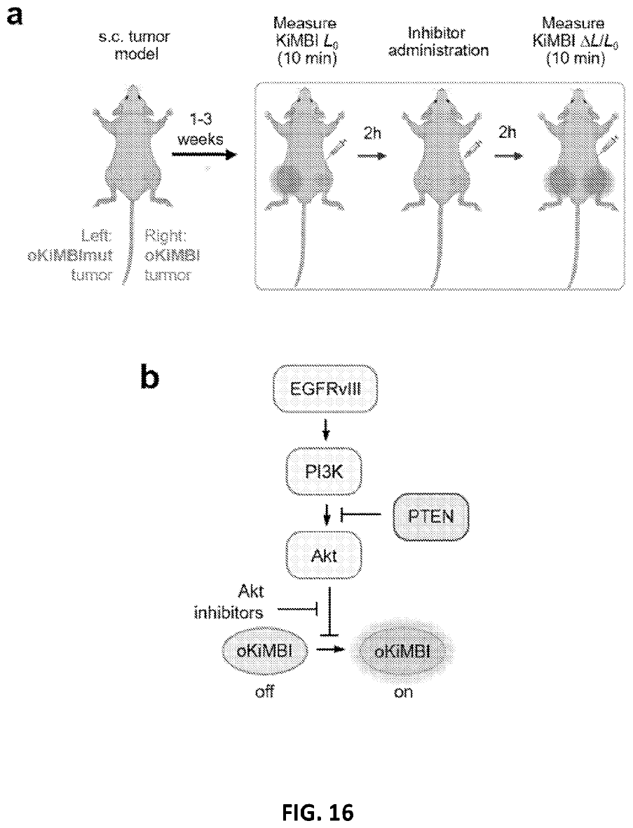

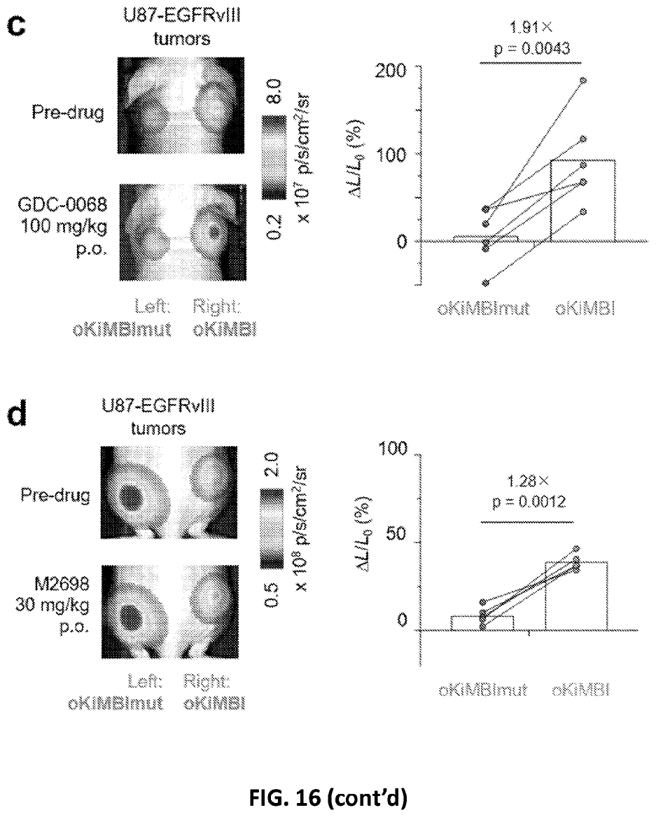

Akt oKiMBI was characterized in various cancer cell lines, including U87-EGFRvIII (human glioma cell line U87 expressing EGFR variant Ill, which activates the PI3K-Akt signaling pathway) and LNCaP (human prostate adenocarcinoma cell line with PTEN loss, thus activating Akt) (FIG. 16b). As anticipated, Akt oKiMBI exhibited a significant signal increase after Akt inhibition in both cell lines. The endogenous Akt activity in these cell lines seemed to be only partially activated, as co-expression of caAkt further enhanced its responsiveness to Akt inhibitor treatment (FIG. 14f). In contrast, the unphosphorylatable mutant with an alanine substitution at the phosphorylation site (Akt oKiMBImut) demonstrated no response to Akt inhibition across any of the tested cell lines (FIG. 14f).

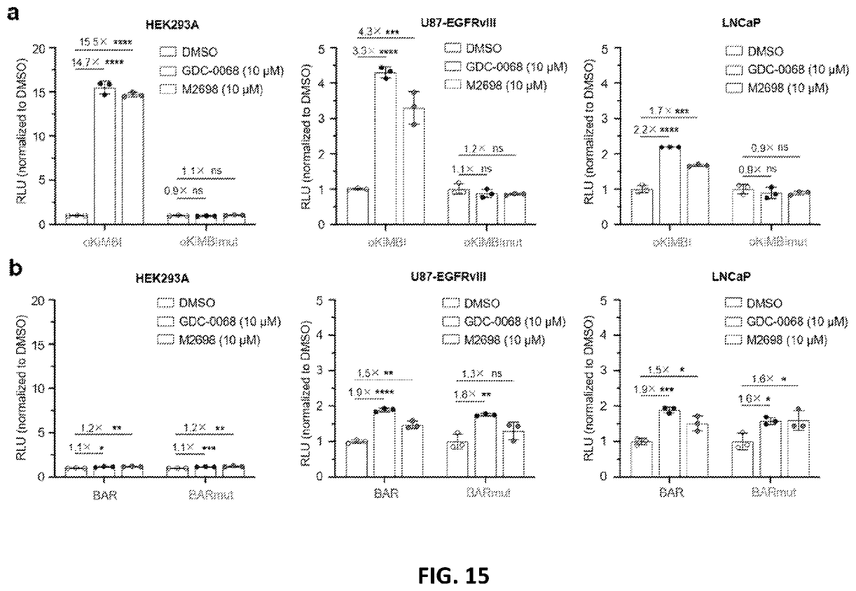

BAR (bioluminescent Akt reporter) is a previously engineered protein that was claimed to report Akt inhibition with increased firefly luciferase (FLuc) light production from D-luciferin⁴. BAR is a fusion protein consisting of, in order, a N-terminal FLuc fragment, a FHA phosphopeptide-binding domain, an Akt substrate sequence, and a C-terminal FLuc fragment. Akt oKiMBI and BAR were tested, along with their respective unphosphorylatable controls, Akt oKiMBImut and BARmut, across various cell lines, including HEK293A (with caAkt co-expression), U87-EGFRvIII, and LNCaP. oKiMBI demonstrated a significant increase in luminescence in response to the treatment of Akt inhibitors GDC-0068 and M2698 in all cell lines, and that oKiMBI_{Akt} mut did not respond to any inhibitor treatment, as expected (FIG. 13c, FIG. 15a). Surprisingly, BAR and BARmut showed miniscule positive responses to GDC-0068 and M2698 across all tested cell lines (FIG. 13d, FIG. 15b). This indicates the BAR response to Akt inhibitors is not due to decreased phosphorylation at the Akt substrate sequence within BAR. As FLuc activity depends on cellular ATP levels, it is possible the observed miniscule response to Akt inhibitors was due to changes in ATP levels in the cell after Akt inhibition. Regardless, the present finding contradicts the previous report indicating a high responsiveness of BAR and lack of response from BARmut following Akt inhibitor treatment.⁴

### Example 7—Molecular Imaging of Akt Inhibition in a Subcutaneous Tumor Xenograft Model

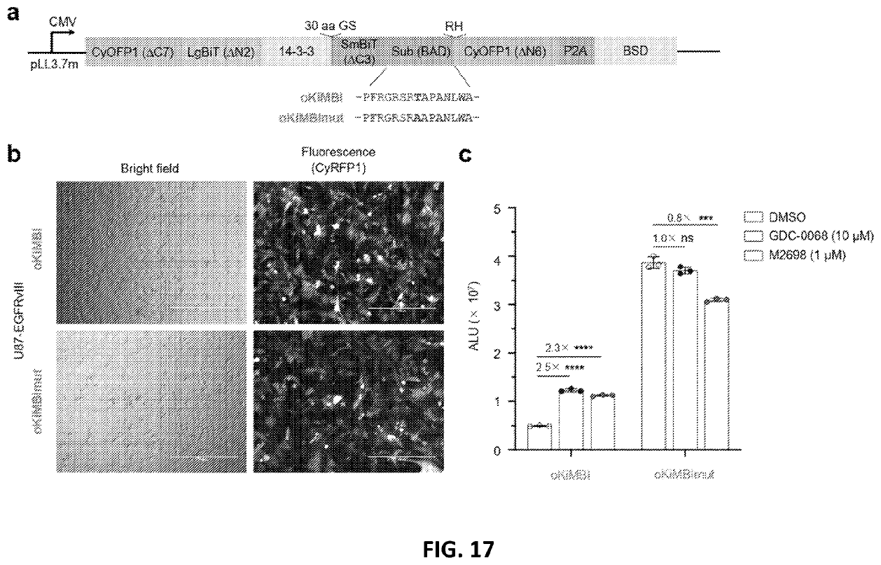

To assess Akt inhibition in human tumor xenografts in mice, either Akt oKiMBI- or oKiMBImut-expressing U87-EGFRvIII stable cell lines were first engineered through lentiviral transduction (FIG. 17a). Fluorescence-activated cell sorting (FACS) was subsequently employed to sort the transduced cells, followed by antibiotics selection to establish polyclonal stable cell lines. The expression of the indicators was verified through fluorescence microscopy (FIG. 17b). The cells stably expressing Akt oKiMBI exhibited an ˜2.5-fold increase in luminescent signals when treated with Akt inhibitors GDC-0068 and M2698, while cells expressing oKiMBImut did not exhibit any signal increase in response to drug treatment (FIG. 17c). The reduced responsiveness of oKiMBI when stably expressed in U87-EGFRvIII cells, compared to its transient expression in U87-EGFRvIII through transfection, is likely attributed to the higher expression of the reporters in the stable cell lines.

Evaluated next was the capacity of Akt oKiMBI to report Akt inhibition in a subcutaneous tumor model. Engineered U87-EGFRvIII cells that stably express oKiMBI or oKiMBImut were subcutaneously injected at different sites in J:NU mice. After an interval of 1-2 weeks post-tumor implantation, bioluminescence imaging was conducted before or 2 hours after the oral administration of Akt inhibitors GDC-0068 (FIG. 16a). Tumors expressing Akt oKiMBI demonstrated a notable increase in signal post-drug treatment, while Akt oKiMBImut-expressing tumors did not exhibit significant signal increase (FIG. 16c).

The non-invasive imaging approach offers a distinct advantage: it enables testing multiple kinase inhibitor candidates within a single mouse through repeated imaging sessions. As an illustration, after allowing a 2-day interval for the injected GDC-0068 to clear, a subsequent test with M2698 on the same group of mice demonstrated effective inhibition of the Akt pathway as well (FIG. 16d). Consequently, Akt oKiMBI facilitates the assessment and comparison of multiple kinase inhibitors in a streamlined manner within a single experimental subject.

### Example 8—Noninvasive Imaging of Akt Inhibitor Activity and Pharmacodynamics in Mouse Brain

Having validated Akt oKiMBI's ability of reporting Akt inhibition in a subcutaneous model, explored next was its potential in visualizing Akt inhibitor activity within mouse brain, thus revealing the BBB permeability of Akt inhibitors.

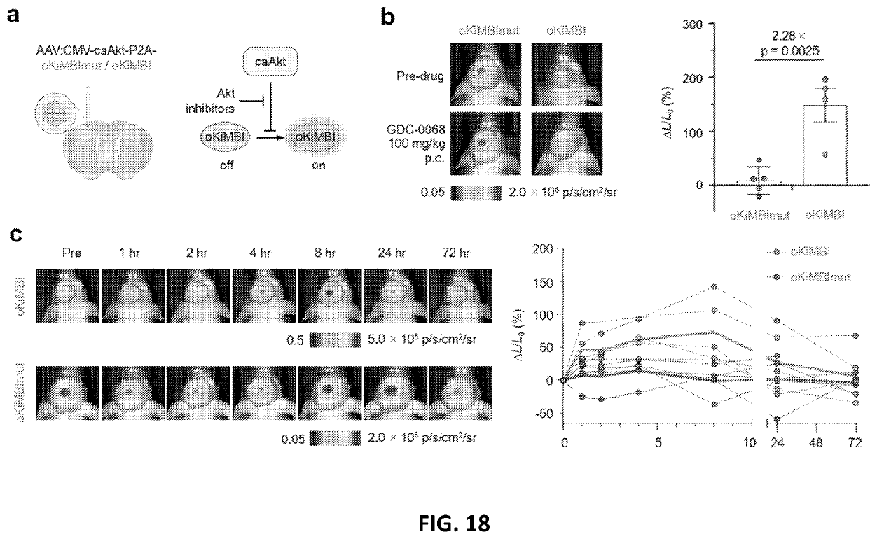

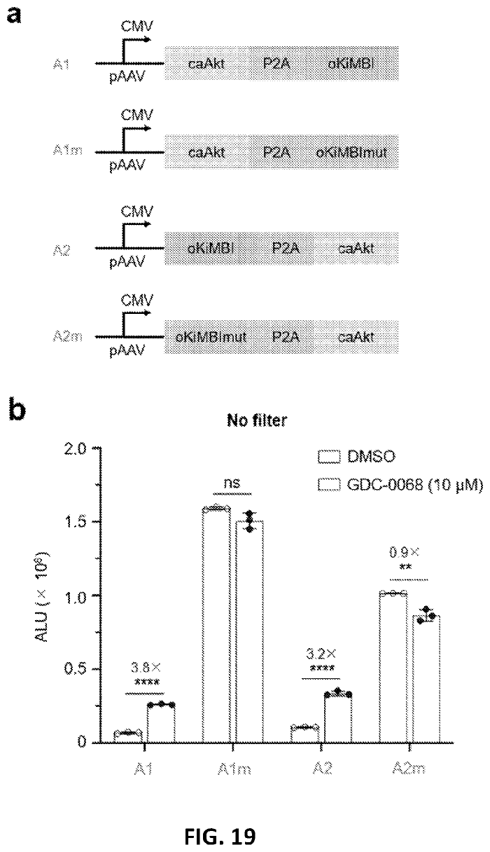

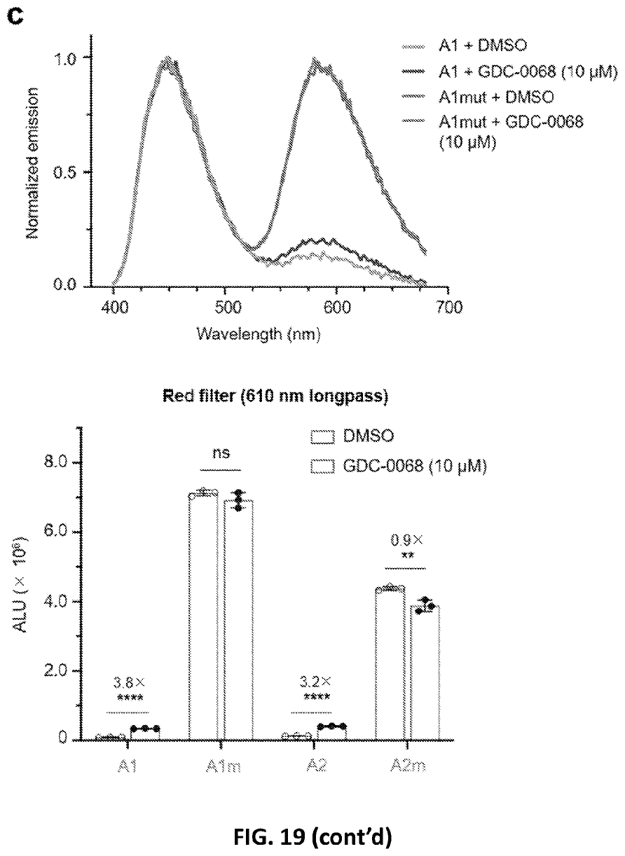

To express Akt oKiMBI in mouse brain, engineered first were adeno-associated viruses (AAVs) that encode Akt oKiMBI (or Akt oKiMBImut) and caAkt, which is used to raise the basal level of Akt activity, mirroring the upregulated Akt activity observed in tumor cells (FIG. 18a). Various domain arrangements were examined, including caAkt-P2A-oKiMBI (A1) and oKiMBI-P2A-caAkt (A2), in HEK293A cells (FIG. 19a). AAV constructs employing both arrangements demonstrated a 3 to 4-fold increase in luminescence upon Akt inhibition, whether with or without a 610 nm longpass filter that simulates an optical window in mouse brain. A1 was selected for further in vivo application due to its relatively higher response compared to A2 (FIG. 19b). The BRET efficiency of Akt oKiMBI and Akt oKiMBImut did not exhibit significant changes with DMSO or GDC-0068 treatment, but Akt oKiMBImut demonstrated notably superior BRET efficiency than oKiMBI (FIG. 19c). This may be attributed to the absence of competition from the 14-3-3-pSub interaction, allowing for the stable formation of the LgBiT-SmBiT complex in Akt oKiMBImut, which, in turn, leads to improved positioning of BRET acceptors relative to the donor, resulting in higher BRET efficiency.

Human tumor xenografts in the mouse brain can potentially yield misleading reports on the efficacy of kinase inhibitors in human patients, because the tumors implanted in mouse brain may display a higher degree of blood-brain barrier (BBB) disruption compared to the human tumors that they are intended to emulate^{1,5}. While GDC-0068 has shown effectiveness in PIK3CA-mutant breast cancer brain metastasis orthotopic xenograft mouse model⁶, its activity within intact brain and its BBB permeability remained unknown.

To address this gap, investigated was the activity of GDC-0068 within the intact mouse brain. AAVs encoding caAkt and Akt oKiMBI were injected into the striatum of J:NU mice (FIG. 18a). After 4 weeks, oKiMBI imaging was conducted both before and 3 hours after the administration of GDC-0068, using the brain-optimized NanoLuc substrate CFz with a rapid signal decay⁷. The negative control mice expressing Akt oKiMBImut did not exhibit a significant signal increase in response to GDC-0068, whereas Akt oKiMBI-expressing mice demonstrated a ˜130% higher response compared to those expressing oKiMBImut after the drug administration (FIG. 18b). These results demonstrate the BBB permeability of GDC-0068.

Furthermore, assessed were the pharmacodynamics of GDC-0068 in mouse brain, which would prove invaluable in establishing the optimal dosing concentration and intervals. Performed was repeated bioluminescence imaging of Akt oKiMBI to visualize the pharmacodynamics of GDC-0068 in mouse brain within the same group of mice. GDC-0068 demonstrated activity in oKiMBI mouse brain as early as one hour after drug administration, and the efficacy persisted for at least 7 hours. In contrast, the negative control mice, expressing Akt oKiMBImut, exhibited a consistent luminescent signal throughout the entire imaging session (FIG. 18c).

### References for Examples 6-8

1. Wu, Y. et al. Kinase-Modulated Bioluminescent Indicators Enable Noninvasive Imaging of Drug Activity in the Brain. ACS Cent Sci 9, 719-732 (2023).
2. Masters, S. C., Yang, H., Datta, S. R., Greenberg, M. E. & Fu, H. 14-3-3 inhibits Bad-induced cell death through interaction with serine-136. Mol Pharmacol 60, 1325-1331 (2001).
3. Tzivion, G., Dobson, M. & Ramakrishnan, G. FoxO transcription factors; Regulation by AKT and 14-3-3 proteins. Biochim Biophys Acta 1813, 1938-1945 (2011).
4. Zhang, L. et al. Molecular imaging of Akt kinase activity. Nat Med 13, 1114-1119 (2007).
5. Brighi, C. et al. Comparative study of preclinical mouse models of high-grade glioma for nanomedicine research: the importance of reproducing blood-brain barrier heterogeneity. Theranostics 10, 6361-6371 (2020).
6. Ippen, F. M. et al. Targeting the PI3K/Akt/mTOR pathway with the pan-Akt inhibitor GDC-0068 in PIK3CA-mutant breast cancer brain metastases. Neuro Oncol 21, 1401-1411 (2019).
7. Su, Y. et al. An optimized bioluminescent substrate for non-invasive imaging in the brain. Nat Chem Biol 19, 731-739 (2023).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A nucleic acid encoding a kinase-modulated bioluminescent indicator (KiMBI) polypeptide, wherein the nucleic acid encodes a KiMBI polypeptide comprising:
a first bioluminescent enzyme fragment;
a phospho-binding domain;
a second bioluminescent enzyme fragment capable of forming an active bioluminescent enzyme with the first fragment via enzyme fragment complementation; and
a kinase substrate which, when phosphorylated, is bound by the phospho-binding domain,
wherein when the kinase substrate is phosphorylated and bound by the phospho-binding domain, the second bioluminescent enzyme fragment adopts an unfavorable confirmation for enzyme fragment complementation with the first bioluminescent enzyme fragment, and
wherein when the kinase substrate is not phosphorylated, the second bioluminescent enzyme fragment adopts a favorable confirmation for enzyme fragment complementation with the first bioluminescent enzyme fragment.

2. The nucleic acid of claim 1, wherein the first bioluminescent enzyme fragment and the second bioluminescent enzyme fragment are fragments of a luciferase.

3. The nucleic acid of claim 2, wherein the luciferase comprises a beta-barrel structure, utilizes coelenterazine, and does not require ATP for catalysis.

4. The nucleic acid of claim 2 or 3, wherein the luciferase is a derivative of a Oplophorus gracilirostris luciferase.

5. The nucleic acid of claim 4, wherein the luciferase is a NanoLuc (NLuc) luciferase.

6. The nucleic acid of claim 2, wherein the luciferase is a Renilla luciferase (RLuc), a Renil/a mutant luciferase (RLuc8), a Gaussia luciferase (GLuc), or a Oplophorus gracilirostris luciferase (OLuc).

7. The nucleic acid of any one of claims 1 to 6, wherein the phospho-binding domain comprises a WW phospho-binding domain or a 14-3-3 phospho-binding domain.

8. The nucleic acid of claim 7, wherein the WW phospho-binding domain is a Pin1 WW phospho-binding domain or variant thereof which retains phospho-binding activity.

9. The nucleic acid of any one of claims 1 to 8, wherein the kinase substrate is a substrate for a tyrosine kinase, external signal-regulated kinase (ERK), mitogen-activated protein kinase kinase (MEK), a cyclin-dependent kinase (CDK), an aurora kinase, a mitogen-activated protein kinase, ERBB1 (EGFR), ERBB2 (HER2), VEGFR, FGFR, Kit, PDGFR, AKT1/AKT2/AKT3, PIK3CA, BRAF, mTOR, SRC, ABL1, BCR-ABL, RET, AATK, EPHA2, EPHA5, CHK2, PKD1, ALK, BTK, MET, KIT, JAK, MEK1/2, Src, ROS1, ROCK, CDK4/CDK6, PI3K %, LRRK2, or combinations thereof.

10. The nucleic acid of claim 9, wherein the kinase substrate is a substrate for ERK.

11. The nucleic acid of claim 10, wherein the substrate is a Cdc25C ERK substrate.

12. The nucleic acid of claim 9, wherein the kinase substrate is a substrate for AKT1/AKT2/AKT3.

13. The nucleic acid of any one of claims 1 to 12, wherein the kinase substrate comprises a single phosphorylation site.

14. The nucleic acid of any one of claims 1 to 12, wherein the kinase substrate comprises two or more phosphorylation sites, optionally wherein the kinase substrate comprises two phosphorylation sites.

15. The nucleic acid of any one of claims 1 to 14, wherein the KiMBI polypeptide comprises, in order from N-terminal to C-terminal: the first bioluminescent enzyme fragment, the phospho-binding domain, the second bioluminescent enzyme fragment, and the kinase substrate.

16. The nucleic acid of any one of claims 1 to 15, wherein the KiMBI polypeptide comprises one or more linkers that link two or more domains of the KiMBI polypeptide.

17. The nucleic acid of any one of claims 1 to 16, wherein the nucleic acid encodes a KiMBI polypeptide further comprising one or more fluorescent protein domains.

18. The nucleic acid of claim 17, wherein the one or more fluorescent protein domains exhibit resonance energy transfer (RET).

19. The nucleic acid of claim 17 or claim 18, wherein the KiMBI polypeptide comprises a fluorescent protein domain at the N-terminus, the C-terminus, or both.

20. The nucleic acid of any one of claims 17 to 19, wherein the one or more fluorescent protein domains are independently selected from a green fluorescent protein (GFP), a yellow fluorescent protein (YFP), a red fluorescent protein (RFP), and an orange fluorescent protein (OFP).

21. The nucleic acid of claim 20, wherein the RFP is a tandem dimer Tomato (tdTomato) RFP.

22. The nucleic acid of claim 20, wherein the OFP is CyOFP1.

23. A KiMBI polypeptide encoded by the nucleic acid of any one of claims 1 to 22.

24. An expression construct comprising the nucleic acid of any one of claims 1 to 22.

25. A cell comprising the nucleic acid of any one of claims 1 to 22.

26. The cell of claim 25, wherein the nucleic acid is present in an expression construct and the cell expresses the KiMBI polypeptide.

27. A non-human animal comprising a plurality of cells of claim 26.

28. The non-human animal of claim 27, wherein the cells are endogenous to the non-human animal such that the expression construct was introduced into the endogenous cells of the non-human animal.

29. The non-human animal of claim 27, wherein the cells are exogenous to the non-human animal such that the cells were introduced into the non-human animal.

30. The non-human animal of any one of claims 27 to 29, wherein the non-human animal is a mammal.

31. The non-human animal of claim 30, wherein the non-human animal is a rodent.

32. The non-human animal of claim 31, wherein the rodent is a mouse.

33. The non-human animal of claim 30, wherein the non-human animal is a non-human primate.

34. A method of assessing activity of a kinase of interest in a living non-human animal, wherein the non-human animal comprises cells that comprise the nucleic acid of any one of claims 1 to 22, the cells co-express the KiMBI polypeptide and the kinase of interest, and the kinase substrate of the KiMBI polypeptide is a substrate for the kinase of interest, the method comprising:
administering to the non-human animal a substrate for the bioluminescent enzyme, wherein the substrate enters the cells co-expressing the KiMBI polypeptide and the kinase of interest; and
assessing the cells for bioluminescence catalyzed by the bioluminescent enzyme, wherein bioluminescence is inversely related to activity of the kinase of interest.

35. A method of assessing a test agent for the ability to inhibit a kinase of interest in a living non-human animal, wherein the non-human animal comprises cells that comprise the nucleic acid of any one of claims 1 to 22, the cells co-express the KiMBI polypeptide and the kinase of interest, and the kinase substrate of the KiMBI polypeptide is a substrate for the kinase of interest, the method comprising:
administering to the non-human animal the test agent;
administering to the non-human animal a substrate for the bioluminescent enzyme, wherein the substrate enters the cells co-expressing the KiMBI polypeptide and the kinase of interest; and
assessing the cells for bioluminescence catalyzed by the bioluminescent enzyme, wherein bioluminescence is positively related to the ability of the test agent to inhibit the kinase of interest.

36. The method according to claim 35, wherein the test agent is a small molecule.

37. The method according to any one of claims 34 to 36, wherein the kinase of interest is a tyrosine kinase, external signal-regulated kinase (ERK), mitogen-activated protein kinase kinase (MEK), a cyclin-dependent kinase (CDK), an aurora kinase, a mitogen-activated protein kinase, ERBB1 (EGFR), ERBB2 (HER2), VEGFR, FGFR, Kit, PDGFR, AKT1/AKT2/AKT3, PIK3CA, BRAF, mTOR, SRC, ABL1, BCR-ABL, RET, AATK, EPHA2, EPHA5, CHK2, PKD1, ALK, BTK, MET, KIT, JAK, MEK1/2, Src, ROS1, ROCK, CDK4/CDK6, PI3Kδ, LRRK2, or combinations thereof.

38. The method according to any one of claims 34 to 37, wherein the substrate for the bioluminescent enzyme is fluorofurimazine (FFz) or cephalofurimazine (CFz).

39. The method according to any one of claims 34 to 38, wherein the cells that co-express the KiMBI polypeptide and the kinase of interest are endogenous to the non-human animal such that the expression construct was introduced into the endogenous cells of the non-human animal.

40. The method according to any one of claims 34 to 38, wherein the cells that co-express the KiMBI polypeptide and the kinase of interest are exogenous to the non-human animal such that the cells were introduced into the non-human animal.

41. The method according to any one of claims 34 to 40, wherein the cells that co-express the KiMBI polypeptide and the kinase of interest are present in the brain of the non-human animal.

42. The method according to any one of claims 34 to 41, wherein the cells that co-express the KiMBI polypeptide and the kinase of interest are tumor cells.

43. The method according to claim 42, wherein the tumor cells are glioblastoma cells.

44. The method according to any one of claims 34 to 43, wherein the cells that co-express the KiMBI polypeptide and the kinase of interest are human cells.
